SEQUENCE LISTING (353 sequences)

SEQ ID NO: 1 - protein, 87 aa, Artificial Sequence
  Synthetic
XIVNVPXXXXXKXXXLXXXAXXXXNXXXXXHLXFVXNXNXKXXXXXXXXYXITLAATDXAGXKXIYXXKXXVVXSXGWXXXXEFXLX

SEQ ID NO: 2 - protein, 87 aa, Artificial Sequence
  Synthetic
XIVNVPNXNXTKXQQLARXAIAXYNXXQNAHLEFVENLNXKXQXXXXXMYYITLAATDDAGKKKIYKAKXXVVXSAGWXXVXEFKLV

SEQ ID NO: 3 - protein, 87 aa, Artificial Sequence
  Synthetic
GIVNVPNXNTTKYQQLARXAIAXYNXXQNAHLEFVENXNXKXQXXXXXMYYITLAATDDAGKKKIYKAKXXVVESAGWXXVXEFKLV

SEQ ID NO: 4 - protein, 100 aa, Artificial Sequence
  Synthetic
XXGMXAISMDTDKISNSFEDCKYFXXXXXSAGXXNTIXNDESGKXEXVXMXVDAIICKNISEENYKXFSKXIEIYHAEGDDVXKNISLXXXGELSKISNP

SEQ ID NO: 5 - protein, 100 aa, Artificial Sequence
  Synthetic
XXGMXAISMDTDKISNSFEDCKYFXXXXXSAGWTNTIXNDESGKXEIVXMXVDAIICKNISEENYKKFSKKIEIYHAEGDDVXKNISLFXEGELSKISNP

SEQ ID NO: 6 - protein, 15 aa, Artificial Sequence
  Synthetic
GXCXXXXSAGWXXCX

SEQ ID NO: 7 - protein, 15 aa, Artificial Sequence
  Synthetic
GXCXGVXSAGWXXCX

SEQ ID NO: 8 - protein, 15 aa, Artificial Sequence
  Synthetic
GXCXGVXSAGWXXCX

SEQ ID NO: 9 - protein, 15 aa, Artificial Sequence
  Synthetic
GXCXGVXSAGWEXCX

SEQ ID NO: 10 - protein, 15 aa, Artificial Sequence
  Synthetic
GXCXXXXSAGWEXCX

SEQ ID NO: 11 - protein, 87 aa, Artificial Sequence
  Synthetic
GIVNVPNXNNTKFQQLARNAIANYNDNQNAHLEFVENLNXKEQVTGGIMYYITLAATDDAGKKKIYKAKIAVVDSAGWEGVTEFKLV

SEQ ID NO: 12 - protein, 87 aa, Artificial Sequence
  Synthetic
GIVNVPNXNNTKFQQLARNAIANYNDYQNAHLEFVENLNXKEQVTGDIMYYITLAATDDAGKKKIYKAKIAVVDSAGWEGVTEFKLV

SEQ ID NO: 13 - protein, 87 aa, Artificial Sequence
  Synthetic
GIVNVPNXNNTKFQQLARYAIANYNDNQNAHLEFVENLNXKEQVTGGIMYYITLAATDDAGKKKIYKAKIAVVDSAGWVGVTEFKLV

SEQ ID NO: 14 - protein, 87 aa, Artificial Sequence
  Synthetic
GIVNVPKXNNTKFQQLARYAIANYNDNQNAHLEFVENLNXKEQVTGGIMYYITLAATDDAGKKKIYKAKIAVVDSAGWVGVTEFKLV

SEQ ID NO: 15 - protein, 87 aa, Artificial Sequence
  Synthetic
GIVNVPNXNTTKYQQLARSAIAIYNYHQNAHLEFVENLNXKEQVGGMNMYYITLAATDDAGKKKIYKAKVGVVESAGWKGVEEFKLV

SEQ ID NO: 16 - protein, 87 aa, Artificial Sequence
  Synthetic
GIVNVPNXNTTKYQQLARSAIAVYNYHQNAHLEFVENLNXKEQIAGKTMYYITLAATDDAGKKKIYKAKVGVVESAGWEGVEEFKLV

SEQ ID NO: 17 - protein, 87 aa, Artificial Sequence
  Synthetic
GIVNVPNXNTTKYQQLARIAIAVYNNYQNAHLEFVENLNXKEQSGGKVMYYITLAATDDAGKKKIYKAKVGVVESAGWIGVEEFKLV

SEQ ID NO: 18 - protein, 87 aa, Artificial Sequence
  Synthetic
GIVNVPNXNTTKYQQLARSAIAVYNNHQNAHLEFVENLNXKEQAGGKAMYYITLAATDDAGKKKIYKAKIGVVESAGWEGVEEFKLV

SEQ ID NO: 19 - protein, 87 aa, Artificial Sequence
  Synthetic
GIVNVPNXNTTKYQQLARRAIAIYNNNQNAHLEFVENLNXKEQTGLGIMYYITLAATDDAGKKKIYKAKIAVVESAGWVAVSEFKLV

SEQ ID NO: 20 - protein, 87 aa, Artificial Sequence; identical to SEQ ID NO: 18
  Synthetic

SEQ ID NO: 21 - protein, 87 aa, Artificial Sequence; identical to SEQ ID NO: 16
  Synthetic

SEQ ID NO: 22 - protein, 87 aa, Artificial Sequence; identical to SEQ ID NO: 18
  Synthetic

SEQ ID NO: 23 - protein, 87 aa, Artificial Sequence; identical to SEQ ID NO: 19
  Synthetic

SEQ ID NO: 24 - protein, 87 aa, Artificial Sequence
  Synthetic
GIVNVPNXNTTKYQQLARRAIAIYNNNQNAHLEFVENLNXKEQWGLGIMYYITLAATDDAGKKKIYKAKIAVVESAGWVAVSEFKLV

SEQ ID NO: 25 - protein, 87 aa, Artificial Sequence
  Synthetic
GIVNVPNXNTTKYQQLARRAIAIYNNNQNAHLEFVENLNXKDQTGLGIMYYITLAATDDAGKKKIYKAKIAVVESAGWVAVSEFKLV

SEQ ID NO: 26 - protein, 87 aa, Artificial Sequence
  Synthetic
GIVNVPNXNTTKYQQLARSAIAIYNNHQNAHLEFVENLNXKEQVSYGAMYYITLAATDDAGKKKIYKAKVGVVESAGWVGVEEFKLV

SEQ ID NO: 27 - protein, 87 aa, Artificial Sequence
  Synthetic
GIVNVPNXNTTKYQQLARSAIAVYNYHQNAHLEFVENLNXKEQIDYGAMYYITLAATDDAGKKKIYKAKIGVVESAGWIGVEEFKLV

SEQ ID NO: 28 - protein, 87 aa, Artificial Sequence; identical to SEQ ID NO: 24
  Synthetic

SEQ ID NO: 29 - protein, 87 aa, Artificial Sequence
  Synthetic
GIVNVPNXNTTKYQQLARSAIAIYNNNQNAHLEFVENLNXKEQLNGYDMYYITLAATDDAGKKKIYKAKIGVVESAGWTGVNEFKLV

SEQ ID NO: 30 - protein, 87 aa, Artificial Sequence
  Synthetic
GIVNVPNXNTTKYQQLARSAIAIYNHYQNAHLEFVENLNXKEQITGYDMYYITLAATDDAGKKKIYKAKVAVVESAGWEVVAEFKLV

SEQ ID NO: 31 - protein, 86 aa, Artificial Sequence
  Synthetic
GIVNVPNXNTTKYQQLARRAIAVYNYYQNAHLEFVENLNXKEQSSGLDMYYITLAATDDAGKKKIYKAKIAVESAGWIVVTEFKLV

SEQ ID NO: 32 - protein, 86 aa, Artificial Sequence
  Synthetic
GIVNVPNXNTTKYQQLARRAIAVYNYYQNAHLEFVENLNXKEQLTGHGMYYITLAATDDAGKKKIYKAKVAVESAGWIVVTEFKLV

SEQ ID NO: 33 - protein, 86 aa, Artificial Sequence
  Synthetic
GIVNVPNXNTTKYQQLARRAIAVYNYYQNAHLEFVENLNXKEQAGGQVMYYITLAATDDAGKKKIYKAKVAVESAGWIVVTEFKLV

SEQ ID NO: 34 - protein, 86 aa, Artificial Sequence
  Synthetic
GIVNVPNXNTTKYQQLARRAIAVYNYYQNAHLEFVENLNXKEQAAGEIMYYITLAATDDAGKKKIYKAKIAVESAGWIVVREFKLV

SEQ ID NO: 35 - protein, 86 aa, Artificial Sequence; identical to SEQ ID NO: 34
  Synthetic

SEQ ID NO: 36 - protein, 87 aa, Artificial Sequence
  Synthetic
GIVNVPNXNTTKYQQLARIAIAIYNYHQNAHLEFVENLNXKEQSGHGTMYYITLAATDDAGKKKIYKAKVGVVESAGWIGVTEFKLV

SEQ ID NO: 37 - protein, 87 aa, Artificial Sequence
  Synthetic
GIVNVPNXNTTKYQQLARIAIAVYNHHQNAHLEFVENLNXKEQISVGTMYYITLAATDDAGKKKIYKAKVGVVESAGWIEVEEFKLV

SEQ ID NO: 38 - protein, 86 aa, Artificial Sequence
  Synthetic
GIVNVPNXNTTKYQQLARRAIAVYNYYQNAHLEFVENLNXKEQLTLGVMYYITLAATDDAGKKKIYKAKVAVESAGWIVVTEFKLV

SEQ ID NO: 39 - protein, 86 aa, Artificial Sequence
  Synthetic
GIVNVPNXNTTKYQQLARRAIAVYNYYQNAHLEFVENLNXKEQSSSGNMYYITLAATDDAGKKKIYKAKIAVESAGWIVVKEFKLV

SEQ ID NO: 40 - protein, 100 aa, Artificial Sequence
  Synthetic
GIGMVAISMDTDKISNSFEDCKYFLIVVVSAGWTNTIFNDESGKKEIVKMNVDAIICKNISEENYKKFSKKIEIYHAEGDDVDKNISLFIEGELSKISNP

SEQ ID NO: 41 - protein, 100 aa, Artificial Sequence
  Synthetic
GIGMVAISMDTDKISNSFEDCKYFLIMVVSAGWTNTISNDESGKKEIVEMNVDAIICKNISEENYKKFSKKIEIYHAEGDDVDKNISLFIEGELSKISNP

SEQ ID NO: 42 - protein, 100 aa, Artificial Sequence
  Synthetic
GIGMVAISMDTDKISNSFEDCKYFLIVVESAGWTNTIFNDESGKKEIVKMNVDAIICKNISEENYKKFSKKIEIYHAEGDDVDKNISLFIEGELSKISNP

SEQ ID NO: 43 - protein, 100 aa, Artificial Sequence
  Synthetic
GTGMVAISMDTDKISNSFEDCKYFLIVMVSAGWTNTIFNDESGKKEIVKMNVDAIICKNISEENYKKFSKKIEIYHAEGDDVDKNISLFIEGELSKISNP

SEQ ID NO: 44 - protein, 100 aa, Artificial Sequence
  Synthetic
GTGMVAISMDTDKISNSFEDCKYFLIMVVSAGWTNTIFNDESGKKEIVEMNVDAIICKNISEENYKKFSKKIEIYHAEGDDVDKNISLFIEGELSKISNP

SEQ ID NO: 45 - protein, 100 aa, Artificial Sequence
  Synthetic
ASGMLAISMDTDKISNSFEDCKYFLIELKSAGWTNTIFNDESGKEEIVKMGVDAIICKNISEENYKKFSKKIEIYHAEGDDVNKNISLFLEGELSKISNP

SEQ ID NO: 46 - protein, 100 aa, Artificial Sequence
  Synthetic
ASGMLAISMDTDKISNSFEDCKYFQLELPSAGWTNTIFNDESGKEEIVKMNVDAIICKNISEENYKKFSKKIEIYHAEGDDVKKNISLFLEGELSKISNP

SEQ ID NO: 47 - protein, 100 aa, Artificial Sequence
  Synthetic
ASGMLAISMDTDKISNSFEDCKYFQIELPSAGGTNTIFNDESGKEEIVKMGVDAIICKNISEENYKKFSKKIEIYHAEGDDVKKNISLFIEGELSKISNP

SEQ ID NO: 48 - protein, 15 aa, Artificial Sequence
  Synthetic
GXCIGVPSAGWEXCW

SEQ ID NO: 49 - protein, 15 aa, Artificial Sequence
  Synthetic
GGCIGVPSAGWEWCP

SEQ ID NO: 50 - protein, 15 aa, Artificial Sequence
  Synthetic
GGCLGVPSAGWEICW

SEQ ID NO: 51 - protein, 15 aa, Artificial Sequence
  Synthetic
GWCIGVPSAGWEICW

SEQ ID NO: 52 - protein, 15 aa, Artificial Sequence
  Synthetic
GRCIGVPSAGWEVCW

SEQ ID NO: 53 - protein, 15 aa, Artificial Sequence
  Synthetic
GHCMGVASAGWEICW

SEQ ID NO: 54 - protein, 15 aa, Artificial Sequence
  Synthetic
GDCIGVASAGWEWCP

SEQ ID NO: 55 - protein, 15 aa, Artificial Sequence
  Synthetic
GGCIGVPSAGWEWCP

SEQ ID NO: 56 - protein, 15 aa, Artificial Sequence
  Synthetic
GGCLGVPSAGWEWCP

SEQ ID NO: 57 - protein, 15 aa, Artificial Sequence
  Synthetic
GWCIGVPSAGWEICW

SEQ ID NO: 58 - protein, 15 aa, Artificial Sequence
  Synthetic
GWCIGVPSAGWEICW

SEQ ID NO: 59 - protein, 15 aa, Artificial Sequence
  Synthetic
GRCIGVPSAGWEVCW

SEQ ID NO: 60 - protein, 15 aa, Artificial Sequence
  Synthetic
GHCMGVASAGWEICW

SEQ ID NO: 61 - protein, 15 aa, Artificial Sequence
  Synthetic
GDCIGVASAGWEWCP

SEQ ID NO: 62 - protein, 15 aa, Artificial Sequence
  Synthetic
GYCIGVPSAGWEVCW

SEQ ID NO: 63 - protein, 15 aa, Artificial Sequence
  Synthetic
GYCIGVPSAGWEICW

SEQ ID NO: 64 - protein, 15 aa, Artificial Sequence
  Synthetic
GWCMGVPSAGWEICW

SEQ ID NO: 65 - protein, 14 aa, Artificial Sequence
  Synthetic
GSCYRVVSAGWETC

SEQ ID NO: 66 - protein, 15 aa, Artificial Sequence
  Synthetic
GDCIVVASAGWEACR

SEQ ID NO: 67 - protein, 15 aa, Artificial Sequence
  Synthetic
GGCARVASAGWEICN

SEQ ID NO: 68 - protein, 15 aa, Artificial Sequence
  Synthetic
GKCRWVASAGWEVCA

SEQ ID NO: 69 - protein, 15 aa, Artificial Sequence
  Synthetic
GNCFAVVSAGWEKCK

SEQ ID NO: 70 - protein, 15 aa, Artificial Sequence
  Synthetic
GMCTHVLSAGWEPCL

SEQ ID NO: 71 - protein, 15 aa, Artificial Sequence
  Synthetic
GMCTHVLSAGWEPCL

SEQ ID NO: 72 - protein, 15 aa, Artificial Sequence
  Synthetic
GCCGYVISAGWEMCS

SEQ ID NO: 73 - protein, 15 aa, Artificial Sequence
  Synthetic
GDCTCMISAGWEPCE

SEQ ID NO: 74 - protein, 15 aa, Artificial Sequence
  Synthetic
GFCCLVTSAGWEECY

SEQ ID NO: 75 - protein, 15 aa, Artificial Sequence
  Synthetic
GFCCLVTSAGWEECY

SEQ ID NO: 76 - protein, 15 aa, Artificial Sequence
  Synthetic
GSCPFVTSAGWEKCL

SEQ ID NO: 77 - protein, 87 aa, Artificial Sequence
  Synthetic
GIVNVPNCNTTKYQQLARTAVAIYNYHEQAHLTFVENLNCKEQLGEGDYYYITLAATDDAGKKAIYEAKLGVVESAGWTGVEEFKLV

SEQ ID NO: 78 - protein, 15 aa, Artificial Sequence
  Synthetic
GDCIVVASAGWEACR

SEQ ID NO: 79 - protein, 87 aa, Artificial Sequence; identical to SEQ ID NO: 77
  Synthetic

SEQ ID NO: 80 - protein, 15 aa, Artificial Sequence
  Synthetic
GRCICALSAGWETCA

SEQ ID NO: 81 - protein, 15 aa, Artificial Sequence
  Synthetic
GWCWGVISAGWEGCR

SEQ ID NO: 82 - protein, 15 aa, Artificial Sequence
  Synthetic
GVCVKVASAGWEECV

SEQ ID NO: 83 - protein, 100 aa, Artificial Sequence; identical to SEQ ID NO: 40
  Synthetic

SEQ ID NO: 84 - protein, 87 aa, Artificial Sequence
  Synthetic
GIVNVPNPNNTKFQQLARNAIANYNDNQNAHLEFVENLNVKEQVTGGIMYYITLAATDDAGKKKIYKAKIAVVDSAGWEGVTEFKLV

SEQ ID NO: 85 - protein, 87 aa, Artificial Sequence
  Synthetic
GIVNVPNPNNTKFQQLARNAIQNYNDNQNAHLEFVENLNVKEQVTGGIMYYITLAATDDAGKKKIYKAKIAVVDSAGWEGITEFKLV

SEQ ID NO: 86 - protein, 87 aa, Artificial Sequence
  Synthetic
GIVNVPNPNNTKFQQLARSAIQNYNDNQNAHLEFVENLNVKEQVTGGIMYYITLAATDDAGKKKIYKAKIAVVDSAGWEGITEFKLV

SEQ ID NO: 87 - protein, 179 aa, Artificial Sequence
  Synthetic
KEITNALETWGALGQDINLDIPSFQMSDDIADIKWEKTQDNKMIVVFSAGWIAKDTYDLYENGTLKIAHLTTDDQAIYKVSITDTKGKNVLEKIFDLKIQERVSKPKISWTCINTTLTCEVMNGTDPELNLYQDGKHLKLSQRVITHKWTTSLSAKFKCTAGNKVSKESSVEPVSCPEK

SEQ ID NO: 88 - protein, 167 aa, Artificial Sequence
  Synthetic
KIIITGEPGVGKTTLVKKIVERLGKRAIGFWTEEVTDPETKKRTGFRIITTEGKKKVFSVVSAGWESKQNFEELAIPILERAYREAKKDRRKVIIIDEIGDALGSSKFRDLVRQIHDPNVNVVATIPIRDDAPLIKEIRRLPGAVLIELTPENRDVILEDILSLLER

SEQ ID NO: 89 - protein, 121 aa, Artificial Sequence
  Synthetic
GHPTLKTPESVTGTWKGDVKIQCIYDPLRGYEQTEVKWLVRHGSDSVTIFERVSSAGWDGISQDKYNGRLQVSDSVPGDVSLQINTLQMDDRNHYTCEVTWQTPDGNQVIRDKIIELRVRK

SEQ ID NO: 90 - protein, 100 aa, Artificial Sequence; identical to SEQ ID NO: 41
  Synthetic

SEQ ID NO: 91 - protein, 100 aa, Artificial Sequence
  Synthetic
GTGMVAISMDTDKISNSFEDCKYFLIVVESAGWTNTIFNDESGKKEIVKMNVDAIICKNISEENYKKFSKKIEIYHAEGDDVDKNISLFIEGELSKISNP

SEQ ID NO: 92 - protein, 102 aa, Artificial Sequence
  Synthetic
GTGMVAISMDTDKISNSFEDCKYFLIVMVSAGWTNTIFNDESGKKEIVKMNVDAIICKNISEENYKKFSKKIEIYHAEGDDVDKNISLFIEGELSKISNPLE

SEQ ID NO: 93 - protein, 102 aa, Artificial Sequence
  Synthetic
GTGMVAISMDTDKISNSFEDCKYFLIMVVSAGWTNTIFNDESGKKEIVEMNVDAIICKNISEENYKKFSKKIEIYHAEGDDVDKNISLFIEGELSKISNPLE

SEQ ID NO: 94 - protein, 87 aa, Artificial Sequence
  Synthetic
GIVNVPNPNNTKFQQLARNAIANYNDYQNAHLEFVENLNVKEQVTGDIMYYITLAATDDAGKKKIYKAKIAVVDSAGWEGVTEFKLV

SEQ ID NO: 95 - protein, 88 aa, Artificial Sequence
  Synthetic
GIVNVPNXNTTKYQQLARIAIAVYNHHQNAHLEFVENLNXVKEQLGEGDMYYITLAATDDAGKKKIYKAKVGVVESAGWTGVEEFKLV

SEQ ID NO: 96 - protein, 88 aa, Artificial Sequence
  Synthetic
GIVNVPNXNTTKYQQLARSAIAIYNYHQNAHLEFVENLNXVKEQLGEGDMYYITLAATDDAGKKKIYKAKIGVVESAGWTGVEEFKLV

SEQ ID NO: 97 - protein, 88 aa, Artificial Sequence
  Synthetic
GIVNVPNXNTTKYQQLARSAIAIYNYHQNAHLEFVENLNXVKEQIGEGSMYYITLAATDDAGKKKIYKAKVGVVESAGWEGVEEFKLV

SEQ ID NO: 98 - protein, 15 aa, Artificial Sequence
  Synthetic
GXCXXVXSAGWEXCX

SEQ ID NO: 99 - protein, 15 aa, Artificial Sequence
  Synthetic
MSMQQVVSAGWERAD

SEQ ID NO: 100 - protein, 13 aa, Artificial Sequence
  Synthetic
MQQVVSAGWERAD

SEQ ID NO: 101 - protein, 19 aa, Artificial Sequence
  Synthetic
HMGXCIGVPSAGWEXCWLE

SEQ ID NO: 102 - protein, 19 aa, Artificial Sequence
  Synthetic
HMGGCIGVPSAGWEWCPLE

SEQ ID NO: 103 - protein, 19 aa, Artificial Sequence
  Synthetic
HMGWCIGVPSAGWEICWLE

SEQ ID NO: 104 - protein, 19 aa, Artificial Sequence
  Synthetic
HMGRCIGVPSAGWEVCWLE

SEQ ID NO: 105 - protein, 19 aa, Artificial Sequence
  Synthetic
HMGHCMGVASAGWEICWLE

SEQ ID NO: 106 - protein, 19 aa, Artificial Sequence
  Synthetic
HMGDCIGVASAGWEWCPLE

SEQ ID NO: 107 - protein, 87 aa, Artificial Sequence
  Synthetic
XIVNVPNPNXTKXQQLARXAIAXYNXXQNAHLEFVENLNVKEQXXXGXMYYITLAATDDAGKKKIYKAKXXVVXSAGWXGVXEFKLV

SEQ ID NO: 108 - protein, 87 aa, Artificial Sequence
  Synthetic
GIVNVPNPNNTKFQQLARNAIANYNQNQNAHLEFVENLNVKEQVTGGIMYYITLAATDDAGKKKIYKAKIAVVDSAGWFGVTEFKLV

SEQ ID NO: 109 - protein, 87 aa, Artificial Sequence
  Synthetic
GIVNVPNPNTTKYQQLARSAIAIYNYHQNAHLEFVENLNVKEQISYGDMYYITLAATDDAGKKKIYKAKIGVVESAGWVGVEEFKLV

SEQ ID NO: 110 - protein, 87 aa, Artificial Sequence; identical to SEQ ID NO: 109
  Synthetic

SEQ ID NO: 111 - protein, 87 aa, Artificial Sequence
  Synthetic
GIVNVPNPNTTKYQQLARRAIAVYNYYQNAHLEFVENLNVKEQISYGSMYYITLAATDDAGKKKIYKAKIGVVESAGWVGVEEFKLV

SEQ ID NO: 112 - protein, 87 aa, Artificial Sequence
  Synthetic
GIVNVPNPNTTKYQQLARSAIAIYNNHQNAHLEFVENLNVKEQVSYGAMYYITLAATDDAGKKKIYKAKVGVVESAGWVGVEEFKLV

SEQ ID NO: 113 - protein, 87 aa, Artificial Sequence
  Synthetic
GIVNVPNPNTTKYQQLARSAIAVYNYHQNAHLEFVENLNVKEQIDYGAMYYITLAATDDAGKKKIYKAKIGVVESAGWIGVEEFKLV

SEQ ID NO: 114 - protein, 87 aa, Artificial Sequence
  Synthetic
MIVNVPNPNTTKYQQLARSAIAVYNHHQNAHLEFVENLNVKEQIGYGXMYYITLAATDDAGKKKIYKAKIGVVESAGWVGVEEFKLV

SEQ ID NO: 115 - protein, 87 aa, Artificial Sequence
  Synthetic
GIVNVPNPNTTKYQQLARSAIAIYNYHQNAHLEFVENLNVKEQIGFGNMYYITLAATDDAGKKKIYKAKVGVVESAGWIGVEEFKLV

SEQ ID NO: 116 - protein, 87 aa, Artificial Sequence
  Synthetic
GIVNVPNPNTTKYQQLARSAIAVYNHYQNAHLEFVENLNVKEQITAGNMYYITLAATDDAGKKKIYKAKIGVVESAGWKGVEEFKLV

SEQ ID NO: 117 - protein, 87 aa, Artificial Sequence
  Synthetic
GIVNVPNPNNTKFQQLARSAIANYNDYQNAHLEFVENLNVKEQVTGDIMYYITLAATDDAGKKKIYKAKIAVVDSAGWEGVTEFKLV

SEQ ID NO: 118 - protein, 87 aa, Artificial Sequence
  Synthetic
GIVNVPNPNNTKFQQLARYAIANYNDYQNAHLEFVENLNVKEQVTGDIMYYITLAATDDAGKKKIYKAKIAVVDSAGWEGVTEFKLV

SEQ ID NO: 119 - protein, 87 aa, Artificial Sequence
  Synthetic
GIVNVPNPNNTKFQQLARNAIANYNDYQNAHLEFVENMNVKEQVTGDIMYYITLAATDDAGKKKIYKAKIAVVDSAGWEGVTEFKLV

SEQ ID NO: 120 - protein, 87 aa, Artificial Sequence
  Synthetic
GIVNVPNPNNTKFQQLARNAIANYNDYQNAHLEFVENLNVKEQVTDDIMYYITLAATDDAGKKKIYKAKIAVVDSAGWEGVTEFKLV

SEQ ID NO: 121 - protein, 87 aa, Artificial Sequence; identical to SEQ ID NO: 117
  Synthetic

SEQ ID NO: 122 - protein, 87 aa, Artificial Sequence; identical to SEQ ID NO: 118
  Synthetic

SEQ ID NO: 123 - protein, 87 aa, Artificial Sequence; identical to SEQ ID NO: 119
  Synthetic

SEQ ID NO: 124 - protein, 87 aa, Artificial Sequence; identical to SEQ ID NO: 120
  Synthetic

SEQ ID NO: 125 - protein, 100 aa, Artificial Sequence
  Synthetic
ASGMLAISMDTDKISNSFEDCKYFQLYVLSAGWTNTIFNDESGKEEIVKMSVDAIICKNISEENYKKFSKKIEIYHAEGDDVDKNISLFLEGELSKISNP

SEQ ID NO: 126 - protein, 87 aa, Artificial Sequence
  Synthetic
GIVNVPNXNTTKYQQLARRAIAIYNHNQNAHLEFVENLNXKEQIDYGSMYYITLAATDDAGKKKIYKAKVGVVESSGWTGVEEFKLV

SEQ ID NO: 127 - protein, 100 aa, Artificial Sequence
  Synthetic
ASGMLAISMDTDKISNSFEDCKYFQIELPSAGWTNTIFNDESGKEEIVKMGVDAIICKNISEENYKKFSKKIEIYHAEGDDVEKNISLFLEGELSKISNP

SEQ ID NO: 128 - protein, 100 aa, Artificial Sequence
  Synthetic
ASGMLAISMDTDKISNSFEDCKYFQLVLASAGWTNTIFNDESGKEEIVKMGVDAIICKNISEENYKKFSKKIEIYHAEGDDVDKNISLFAEGELSKISNP

SEQ ID NO: 129 - protein, 87 aa, Artificial Sequence
  Synthetic
GIVNVPNPNNTKFQELARFAIQDYNKKQNAHLEFVENLNVKEQVVAGIMYYITLAATDDAGKKKIYKAKIWVKEWEDFKKVVEFKLV

SEQ ID NO: 130 - protein, 104 aa, Artificial Sequence
  Synthetic
MINMKVAISMDVDKISNSFEDCKYFLIVRIDDNEVKSTKVIFNDESGKKSIVKENVNAIICKNISEENYKKFSKKIEIYHAEGDDVDKNISLFIEGELSKISNP

SEQ ID NO: 131 - protein, 87 aa, Artificial Sequence
  Synthetic
GIVNVPNXNNTKFQQLARSAIANYNDYQNAHLEFVENLNXKEQVTGDIMYYITLAATDDAGKKKIYKAKIAVVDSAGWEGVTEFKLV

SEQ ID NO: 132 - protein, 87 aa, Artificial Sequence
  Synthetic
GIVNVPNXNNTKFQQLARYAIANYNDYQNAHLEFVENLNXKEQVTGDIMYYITLAATDDAGKKKIYKAKIAVVDSAGWEGVTEFKLV

SEQ ID NO: 133 - protein, 87 aa, Artificial Sequence
  Synthetic
GIVNVPNXNNTKFQQLARNAIANYNDYQNAHLEFVENMNXKEQVTGDIMYYITLAATDDAGKKKIYKAKIAVVDSAGWEGVTEFKLV

SEQ ID NO: 134 - protein, 87 aa, Artificial Sequence
  Synthetic
GIVNVPNXNNTKFQQLARNAIANYNDYQNAHLEFVENLNXKEQVTDDIMYYITLAATDDAGKKKIYKAKIAVVDSAGWEGVTEFKLV

SEQ ID NO: 135 - protein, 87 aa, Artificial Sequence
  Synthetic
GIVNVPNCNTTKYQQLARTAVAIYNYHEQAHLTFVENLNCKEQLGEGDYYYITLAATDDAGKKAIYEAKIGVVESAGWTGVEEFKLV

SEQ ID NO: 136 - protein, 87 aa, Artificial Sequence
  Synthetic
GIVNVPNCNTTKYQQLARTAIAIMNYHQQAHLTFVENLNCKEQLGEGDYYYITLAATDDAGKKAIYEAKIGVVESAGWTGVEEFKLV

SEQ ID NO: 137 - protein, 87 aa, Artificial Sequence
  Synthetic
GIVNVPNCNTTKYQQLARTAIAIVNCHEHAHLTFVENLNCKEQLGEGDIYYITLAATDDAGKKSIYEAKIGVVESAGWTGVEEFKLV

SEQ ID NO: 138 - protein, 87 aa, Artificial Sequence
  Synthetic
GIVNVPNCNTTKYQQLARTAVAIYNYHKPAHLTFVENLNCKEQLGEGDIYYITLAATDDAGKKSIYSAKIGVVESAGWTGVEEFKLV

SEQ ID NO: 139 - protein, 87 aa, Artificial Sequence
  Synthetic
GIVNVPNCNTTKYQQLARTAVAIYNYHNHAHLTFVENLNCKEQLGEGDLYYITLAATDDAGKKAIYTAKIGVVESAGWTGVEEFKLV

SEQ ID NO: 140 - protein, 87 aa, Artificial Sequence
  Synthetic
GIVNVPNPNTTKYQQLARTAVAIYNYHEQAHLCFVENLNVKEQLGEGDYYYITLAACDDAGKKAIYEAKIGVVESAGWTGVEEFKLV

SEQ ID NO: 141 - protein, 84 aa, Artificial Sequence
  Synthetic
GIVNVPNCNTTKYQQLARTAVAIYNYHEQAHLTFVENLNCKEQGNYYYITLAATDDAGKKAIYEAKIGVVESAGWTGVEEFKLV

SEQ ID NO: 142 - protein, 84 aa, Artificial Sequence
  Synthetic
GIVNVPNPNTTKYQQLARTAVAIYNYHEQAHLCFVENLNVKEQGNYYYITLAACDDAGKKAIYEAKIGVVESAGWTGVEEFKLV

SEQ ID NO: 143 - protein, 87 aa, Artificial Sequence
  Synthetic
GIVNVPNCNTTKYQQLARTAVAIYNYHEQAHLTFVENLNCKEQLGEGDYYYITLAATDDAGKKWIYEAKIGVVESAGWTGVEEFKLV

SEQ ID NO: 144 - protein, 87 aa, Artificial Sequence
  Synthetic
GIVNVPNCNTTKYQQLARTAVAIYNYHEPAHLTFVENLNCKEQLGEGDYYYITLAATDDAGKKAIYEAKIGVVESAGWTGVEEFKLV

SEQ ID NO: 145 - protein, 87 aa, Artificial Sequence
  Synthetic
GIVNVPNCNTTKYRQLARTAVAIYNYHEQAHLTFVENLNCKEQLGEGDYYYITLAATDDAGKKAIYEAKIGVVESAGWTGVEEFKLV

SEQ ID NO: 146 - protein, 87 aa, Artificial Sequence
  Synthetic
GIVNVPNCNTTKYQQLARTAVAIYNYHEQAHLTFVENLNCKEKLGEGDYYYITLAATDDAGKKAIYEAKIGVVESAGWTGVEEFKLV

SEQ ID NO: 147 - protein, 87 aa, Artificial Sequence
  Synthetic
GIVNVPNCNTTKYQQLARTAVAIYNYHEQAHLTFVENLNCKEQLGEGDYYYITLAATDDAGKKAIYETKIGVVESAGWTGVEEFKLV

SEQ ID NO: 148 - protein, 87 aa, Artificial Sequence
  Synthetic
GIVNVPNCNTKKYQQLARTAVAIYNYHEQAHLTFVENLNCKEQLGEGDYYYITLAATDDAGKKAIYEAKIGVVESAGWTGVEEFKLV

SEQ ID NO: 149 - protein, 87 aa, Artificial Sequence
  Synthetic
GIVNVPNCNTTKYQQLARTAVAIYNYHEQAHLTFVENLNCKEQLGEGDYYYITLAATDQAGKKAIYEAKIGVVESAGWTGVEEFKLV

SEQ ID NO: 150 - protein, 87 aa, Artificial Sequence
  Synthetic
GIVNVPNCNTTKYQQLARTAVAIYNYHEQAHLTFVENLNCKEQLGEGDLYYITLAATDDAGKKAIYEAKIGVVESAGWTGVEEFKLV

SEQ ID NO: 151 - protein, 87 aa, Artificial Sequence
  Synthetic
GIVNVPNCNTTKYQQLARTAVAIYNYHEQAHLYFVENLNCKEQLGEGDYYYITLAATDDAGKKAIYEAKIGVVESAGWTGVEEFKLV

SEQ ID NO: 152 - protein, 87 aa, Artificial Sequence
  Synthetic
GIVNVPNCNTTKYQQLARTAVAIYNYHEQAHLNFVENLNCKEQLGEGDYYYITLAATDDAGKKAIYEAKIGVVESAGWTGVEEFKLV

SEQ ID NO: 153 - protein, 87 aa, Artificial Sequence
  Synthetic
GIVNVPNCNTTKYQQLARTAVAIYNYHEQAHLTFVENLNCKEQLGEGDYYWITLAATDDAGKKAIYEAKIGVVESAGWTGVEEFKLV

SEQ ID NO: 154 - protein, 87 aa, Artificial Sequence
  Synthetic
GIVNVPNCNTTKYQQLARTAVAIYNYHEQAHLAFVENLNCKEQLGEGDYYYITLAATDDAGKKAIYEAKIGVVESAGWTGVEEFKLV

SEQ ID NO: 155 - protein, 87 aa, Artificial Sequence
  Synthetic
GIVNVPNCNTTKYQQLARTAVAIYNYHEQAHLTFVENLNCKEQLGEGDYYYITLAATDDAGKKAIYEAKIGVVESAGWTGVEEFKLI

SEQ ID NO: 156 - protein, 87 aa, Artificial Sequence
  Synthetic
GIVNVPNCNTTKYQQLARTAVAIYNYHEQAHLTFVENLNCKEQLGEGDYYYITLAATDDAGKKAIYEAKIGVVESAGWTGVEEFALV

SEQ ID NO: 157 - protein, 87 aa, Artificial Sequence
  Synthetic
GIVNVPNCNTTKYIQLARTAVAIYNYHEQAHLTFVENLNCKEQLGEGDYYYITLAATDDAGKKAIYEAKIGVVESAGWTGVEEFKLV

SEQ ID NO: 158 - protein, 87 aa, Artificial Sequence
  Synthetic
GIVNVPNCNTTKYQQLARTAVAIYNYHEQAHLTFVENLNCKEQLGEKDYYYITLAATDDAGKKAIYEAKIGVVESAGWTGVEEFKLV

SEQ ID NO: 159 - protein, 87 aa, Artificial Sequence
  Synthetic
GIVNVPNCNTTKYQQLARTAVAIYNYHEQAHLTFVENLNCKEQLGEGDYYYITLAATDDAGEKAIYEAKIGVVESAGWTGVEEFKLV

SEQ ID NO: 160 - protein, 87 aa, Artificial Sequence; identical to SEQ ID NO: 135
  Synthetic

SEQ ID NO: 161 - protein, 87 aa, Artificial Sequence
  Synthetic
GIVNVPNCNTTKYQQLARTAVAIYNYHDQAHLTFVENLNCKEQLGEGDYYYITLAATDDAGKKAIYEAKIGVVESAGWTGVEEFKLV

SEQ ID NO: 162 - protein, 87 aa, Artificial Sequence
  Synthetic
GIVNVPNCNTTKYQQLARTAVAIYNYHEQAHLTFVENLNCKEQLGEGDYYYITLAATDDAGKKAIYSAKIGVVESAGWTGVEEFKLV

SEQ ID NO: 163 - protein, 87 aa, Artificial Sequence
  Synthetic
GIVNVPNCNTTKYQQLARTAVAIYNYHEQAHLTFVENLNCKEQLGEGDYYYITLAATDDAGKKAIYEAKIGVVESAGWTGVEEFELV

SEQ ID NO: 164 - protein, 87 aa, Artificial Sequence
  Synthetic
GIVNVPNCNTTKYQQLARTAVAIYNYHEQAHLTFVENLNCKEQLGEGDYYYITLAATDDAGKKAIYAAKIGVVESAGWTGVEEFKLV

SEQ ID NO: 165 - protein, 87 aa, Artificial Sequence
  Synthetic
GIVNVPNCNTTKYQQLARTAVAIYNYHEQAHLTFVHNLNCKEQLGEGDYYYITLAATDDAGKKAIYEAKIGVVESAGWTGVEEFKLV

SEQ ID NO: 166 - protein, 87 aa, Artificial Sequence
  Synthetic
GIVNVPNCNTTKYQQLARTAVAIYNYHEQAHLTFVENLNCKEQLGEGDYYYITLAATDDAGKKAIYEAKIGVVESAGWTGLEEFKLV

SEQ ID NO: 167 - protein, 87 aa, Artificial Sequence
  Synthetic
GIVNVPNCNTTKYQQLARTAVAIYNYHEQEHLTFVENLNCKEQLGEGDYYYITLAATDDAGKKAIYEAKIGVVESAGWTGVEEFKLV

SEQ ID NO: 168 - protein, 87 aa, Artificial Sequence
  Synthetic
GIVNVPNCNTPKYQQLARTAVAIYNYHEQAHLTFVENLNCKEQLGEGDYYYITLAATDDAGKKAIYEAKIGVVESAGWTGVEEFKLV

SEQ ID NO: 169 - protein, 87 aa, Artificial Sequence
  Synthetic
GIVNVPNCNTTKYQQLARTAVAIYNYHEQAHLTFVENLNCKEQLGEGDYYYITLAATDDAGKKMIYEAKIGVVESAGWTGVEEFKLV

SEQ ID NO: 170 - protein, 87 aa, Artificial Sequence
  Synthetic
GIVNVPNCNTTKYQQLARTAVAIYNYHEQAHLTFVENLNCKEQLGEGDYYYITLAATDDAGKKSIYEAKIGVVESAGWTGVEEFKLV

SEQ ID NO: 171 - protein, 87 aa, Artificial Sequence
  Synthetic
GIVNVPNCNTTKYQQLARTAVAIYNYHKQAHLTFVENLNCKEQLGEGDYYYITLAATDDAGKKAIYEAKIGVVESAGWTGVEEFKLV

SEQ ID NO: 172 - protein, 87 aa, Artificial Sequence
  Synthetic
GIVNVPNCNTTKYQQLARTAVSIYNYHEQAHLTFVENLNCKEQLGEGDYYYITLAATDDAGKKAIYEAKIGVVESAGWTGVEEFKLV

SEQ ID NO: 173 - protein, 87 aa, Artificial Sequence
  Synthetic
GIVNVPNCNTTKYQQLARTAVAIYNYDEQAHLTFVENLNCKEQLGEGDYYYITLAATDDAGKKAIYEAKIGVVESAGWTGVEEFKLV

SEQ ID NO: 174 - protein, 87 aa, Artificial Sequence
  Synthetic
GIVNVPNCNTTKYQQLARTAVAIYNYHEQAHLLFVENLNCKEQLGEGDYYYITLAATDDAGKKAIYEAKIGVVESAGWTGVEEFKLV

SEQ ID NO: 175 - protein, 87 aa, Artificial Sequence
  Synthetic
GIVNVPNCNTTKYQQLARTAVAIYNYHEQAHLIFVENLNCKEQLGEGDYYYITLAATDDAGKKAIYEAKIGVVESAGWTGVEEFKLV

SEQ ID NO: 176 - protein, 87 aa, Artificial Sequence
  Synthetic
GIVNVPNCNTTKYQQLARTAVAIYNYHWQAHLTFVENLNCKEQLGEGDYYYITLAATDDAGKKAIYEAKIGVVESAGWTGVEEFKLV

SEQ ID NO: 177 - protein, 87 aa, Artificial Sequence
  Synthetic
GIVNVPNCNTTKYQQLARTAVAIYNYHEQAHLTFVENLNCKEQLGEGDYYYITLAATDDAGKKQIYEAKIGVVESAGWTGVEEFKLV

SEQ ID NO: 178 - protein, 87 aa, Artificial Sequence
  Synthetic
GIVNVPNCNTTKYQQLARTAVAIYNYHEQAHLTFVENLNCKEQLGEGDYYYITLAATDDAGKKAIYYAKIGVVESAGWTGVEEFKLV

SEQ ID NO: 179 - protein, 87 aa, Artificial Sequence
  Synthetic
GIVNVPNCNTTKYQQLARTAVYIYNYHEQAHLTFVENLNCKEQLGEGDYYYITLAATDDAGKKAIYEAKIGVVESAGWTGVEEFKLV

SEQ ID NO: 180 - protein, 87 aa, Artificial Sequence
  Synthetic
GIVNVPNCNTTKYQQLARTAVAIYNYHEQAHLTFVENLNCKEQLGEGDYYYITLAATDDAGKKGIYEAKIGVVESAGWTGVEEFKLV

SEQ ID NO: 181 - protein, 87 aa, Artificial Sequence
  Synthetic
GIVNVPNCNTTKYQQLARTAVAIYNYHEQAHLTFVENLNCKEQLGEGDYYYITLAATDDAGKKAIYWAKIGVVESAGWTGVEEFKLV

SEQ ID NO: 182 - protein, 87 aa, Artificial Sequence
  Synthetic
GIVNVPNCNTTKYQQLARTAVAIYNYHEQAHLTFVENLNCKEQLGEGDYYYITLAATDDAGKKAIYMAKIGVVESAGWTGVEEFKLV

SEQ ID NO: 183 - protein, 87 aa, Artificial Sequence
  Synthetic
GIVNVPNCNTTKYQQLARTAVAIYNYHEDAHLTFVENLNCKEQLGEGDYYYITLAATDDAGKKAIYEAKIGVVESAGWTGVEEFKLV

SEQ ID NO: 184 - protein, 87 aa, Artificial Sequence
  Synthetic
GIVNVPNCNTTKYQQLARTAVAIYNYHEQAHLHFVENLNCKEQLGEGDYYYITLAATDDAGKKAIYEAKIGVVESAGWTGVEEFKLV

SEQ ID NO: 185 - protein, 87 aa, Artificial Sequence
  Synthetic
GIVNVPNCNTTKYQQLARTAVAIYNYHEQAHLTFVENLNCKEQLGEGDYYYITLAATDDAGKKAIYEAKIGVVESAGWTGVEEFDLV

SEQ ID NO: 186 - protein, 87 aa, Artificial Sequence
  Synthetic
GIVNVPNCNTTKYQQLARTAVAIMNYHEQAHLTFVENLNCKEQLGEGDYYYITLAATDDAGKKAIYEAKIGVVESAGWTGVEEFKLV

SEQ ID NO: 187 - protein, 87 aa, Artificial Sequence; identical to SEQ ID NO: 135
  Synthetic

SEQ ID NO: 188 - protein, 87 aa, Artificial Sequence; identical to SEQ ID NO: 135
  Synthetic

SEQ ID NO: 189 - protein, 87 aa, Artificial Sequence
  Synthetic
GIVNVPNCNTTKYQQLARTAVAIYNYHEQAHLTFVENLNCKEQLGEGDYYYITLAATDDAGKKAIYEAKIGVVESAGWLGVEEFKLV

SEQ ID NO: 190 - protein, 87 aa, Artificial Sequence
  Synthetic
GIVNVPNCNTTKYQQLARTAVAIYNYHEQAHLSFVENLNCKEQLGEGDYYYITLAATDDAGKKAIYEAKIGVVESAGWTGVEEFKLV

SEQ ID NO: 191 - protein, 87 aa, Artificial Sequence
  Synthetic
GIVNVPNCNTTKYQQLARTAVAIWNYHEQAHLTFVENLNCKEQLGEGDYYYITLAATDDAGKKAIYEAKIGVVESAGWTGVEEFKLV

SEQ ID NO: 192 - protein, 87 aa, Artificial Sequence
  Synthetic
GIVNVPNCNTTKYQQLARTAVAIINYHEQAHLTFVENLNCKEQLGEGDYYYITLAATDDAGKKAIYEAKIGVVESAGWTGVEEFKLV

SEQ ID NO: 193 - protein, 87 aa, Artificial Sequence
  Synthetic
GIVNVPNCNTTKYQQLARQAVAIYNYHEQAHLTFVENLNCKEQLGEGDYYYITLAATDDAGKKAIYEAKIGVVESAGWTGVEEFKLV

SEQ ID NO: 194 - protein, 87 aa, Artificial Sequence
  Synthetic
GIVNVPNCNTTKYQQLARTAVAIVNYHEQAHLTFVENLNCKEQLGEGDYYYITLAATDDAGKKAIYEAKIGVVESAGWTGVEEFKLV

SEQ ID NO: 195 - protein, 87 aa, Artificial Sequence
  Synthetic
GIVNVPNCNTTKYQQLARTAVAIYNYHEQAHLFFVENLNCKEQLGEGDYYYITLAATDDAGKKAIYEAKIGVVESAGWTGVEEFKLV

SEQ ID NO: 196 - protein, 87 aa, Artificial Sequence
  Synthetic
GIVNVPNCNTTKYQQLARTAVAIYNYHEQAHLMFVENLNCKEQLGEGDYYYITLAATDDAGKKAIYEAKIGVVESAGWTGVEEFKLV

SEQ ID NO: 197 - protein, 87 aa, Artificial Sequence
  Synthetic
GIVNVPNCNTTKYQKLARTAVAIYNYHEQAHLTFVENLNCKEQLGEGDYYYITLAATDDAGKKAIYEAKIGVVESAGWTGVEEFKLV

SEQ ID NO: 198 - protein, 87 aa, Artificial Sequence
  Synthetic
GIVNVPNCNTTKYQQLARTAVAIYNYHEQAHLVFVENLNCKEQLGEGDYYYITLAATDDAGKKAIYEAKIGVVESAGWTGVEEFKLV

SEQ ID NO: 199 - protein, 87 aa, Artificial Sequence
  Synthetic
GIVNVPNCNTTKYQQLARTAVAIYNYHEQAHLTFVQNLNCKEQLGEGDYYYITLAATDDAGKKAIYEAKIGVVESAGWTGVEEFKLV

SEQ ID NO: 200 - protein, 102 aa, Artificial Sequence
  Synthetic
MEEVVLITVPSEEVARTIAKALVEERLAACVNIVPGLTSIYRWQGEVVEDQELLLLVKTTTHAFPKLKERVKALHPYTVPEIVALPIAQGNQEYLDWLRENT

SEQ ID NO: 201 - protein, 101 aa, Artificial Sequence
  Synthetic
MEEVVLITVPSEEVARTIAKALVEERLAACVNIVPGLTSIYRWQGEVVEDQELLLLVKTTTAAFPKLKERVKALHPYTVPEIVALPIAEGNREYLDWLQEN

SEQ ID NO: 202 - protein, 110 aa, Artificial Sequence
  Synthetic
ALYFSGHMILVYSTFPNEEKALEIGRKLLEKRLIACFNAFEIRSGYWWKGEIVQDKEWAAIFKTTQSKQKELYEELRKLHPYETPAIFTLKVENILTEYMNWLAESVLGS

SEQ ID NO: 203 - protein, 93 aa, Artificial Sequence
  Synthetic
MKLIVAIVRPEKLNEVLKALFQAEVRGLTLSRVQGHGMELHEKVRLEIGVSEPFVKPTVEAILKAARTGEVGDGKIFVLPVEKVYRIRTGEEA

SEQ ID NO: 204 - protein, 108 aa, Artificial Sequence
  Synthetic
MKLIVAIVRPEKLNEVLKALFQAEVRGLTLSRVQGHGGETERVETYRGTTVKMELHEKVRLEIGVSEPFVKPTVEAILKAARTGEVGDGKIFVLPVEKVYRIRTGEEA

SEQ ID NO: 205 - protein, 202 aa, Artificial Sequence
  Synthetic
KMEELFKKHKIVAVLRANSVEEAKEKALAVFEGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGQVVGPQFVKAMKGPFPNVKFVPTGGVNLSNVAAWFAAGVLAVGVGSALVKGTPNNVREKAKAFVEAIRGA

SEQ ID NO: 206 - protein, 148 aa, Artificial Sequence
  Synthetic
KDSEIVKALGDLDELNSVLGVVSSLYPSLSQVIQKLQNDIFSISSEIAGFDMNFSDEKVKGIEELITNYSKELEPLSNFVLPGGHIASSFLHLARAVCRRAERSVVTLLKESKAKEVHAKYLNRLSSLLFVLALVVNKRTSNPNVIWR

SEQ ID NO: 207 - protein, 142 aa, Artificial Sequence
  Synthetic
SKDSPIIEANGTLDELTSFIGEAKHYVDAEMQGILEEIQNDIYKIMGEIGSKGKIEGISEERIKWLEGLISRYEEMVNLKSFVLPGGTLESAKLDVCRTIARRAERKVATVLREFGIGKEALVYLNRLSDLLFLLARVIEIE

SEQ ID NO: 208 - protein, 117 aa, Artificial Sequence
  Synthetic
GSMKKVEAIIRPEKLEIVKKALSDAGYVGMTVSEVKGRGVQGGIVERYRGREYIVDLIPKVKIELVVKQADVDNVIDIICENARTGNPGDGKIFVIPVQRVVRVRTKEEGAAALLEH

SEQ ID NO: 209 - protein, 99 aa, Artificial Sequence
  Synthetic
ILVYSTFPNEEKALEIGRKLLEKRLIACFNAFEIRSGYWWKGEIVQDKEWAAIFKTTEEKEKELYEELRKLHPYETPAIFTLKVENVLTEYMNWLRESV

SEQ ID NO: 210 - protein, 114 aa, Artificial Sequence
  Synthetic
GSMKKVEAIIRPEKLEIVKKALSDAGYVGMTVSEVKGRGVQGGIVERYRGREYIVDLIPKVKIELVVKEEDVDNVIDIICENARTGNPGDGKIFVIPVERVVRVRTKEEGKEAL

SEQ ID NO: 211 - protein, 105 aa, Artificial Sequence
  Synthetic
KKIEAIIRPFKLDEVKIALVNAGIVGMTVSEVRGFGRQKRGSEYTVEFLQKLKLEIVVEDAQVDTVIDKIVAAARTGENGDGKIFVSPVDQTIRIRTGEKNADAI

SEQ ID NO: 212 - protein, 98 aa, Artificial Sequence
  Synthetic
HLTPREFDKLVIHMLSDVALKRKNKGLKLNHPEAVAVLSAYVLDGAREGKTVEEVMDGARSVLKADDVMDGVPDLLPLIQVEAVFSDGSRLVSLHNPI

SEQ ID NO: 213 - protein, 104 aa, Artificial Sequence
  Synthetic
MEEVVLITVPSEEVARTIAKALVEERLAACVNIVPGLTSIYRWQGEVVEDQELLLLVKTTTHAFPKLKERVKALHPYTVPEIVALPIAQGNQEYLDWLRENTGS

SEQ ID NO: 214 - protein, 103 aa, Artificial Sequence
  Synthetic
MEEVVLITVPSEEVARTIAKALVEERLAACVNIVPGLTSIYRWQGEVVEDQELLLLVKTTTAAFPKLKERVKALHPYTVPEIVALPIAEGNREYLDWLQENGS

SEQ ID NO: 215 - protein, 112 aa, Artificial Sequence
  Synthetic
ALYFSGHMILVYSTFPNEEKALEIGRKLLEKRLIACFNAFEIRSGYWWKGEIVQDKEWAAIFKTTQSKQKELYEELRKLHPYETPAIFTLKVENILTEYMNWLAESVLGSGS

SEQ ID NO: 216 - protein, 95 aa, Artificial Sequence
  Synthetic
MKLIVAIVRPEKLNEVLKALFQAEVRGLTLSRVQGHGMELHEKVRLEIGVSEPFVKPTVEAILKAARTGEVGDGKIFVLPVEKVYRIRTGEEAGS

SEQ ID NO: 217 - protein, 110 aa, Artificial Sequence
  Synthetic
MKLIVAIVRPEKLNEVLKALFQAEVRGLTLSRVQGHGGETERVETYRGTTVKMELHEKVRLEIGVSEPFVKPTVEAILKAARTGEVGDGKIFVLPVEKVYRIRTGEEAGS

SEQ ID NO: 218 - protein, 204 aa, Artificial Sequence
  Synthetic
KMEELFKKHKIVAVLRANSVEEAKEKALAVFEGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGQVVGPQFVKAMKGPFPNVKFVPTGGVNLSNVAAWFAAGVLAVGVGSALVKGTPNNVREKAKAFVEAIRGAGS

SEQ ID NO: 219 - protein, 150 aa, Artificial Sequence
  Synthetic
KDSEIVKALGDLDELNSVLGVVSSLYPSLSQVIQKLQNDIFSISSEIAGFDMNFSDEKVKGIEELITNYSKELEPLSNFVLPGGHIASSFLHLARAVCRRAERSVVTLLKESKAKEVHAKYLNRLSSLLFVLALVVNKRTSNPNVIWRGS

SEQ ID NO: 220 - protein, 144 aa, Artificial Sequence
  Synthetic
SKDSPIIEANGTLDELTSFIGEAKHYVDAEMQGILEEIQNDIYKIMGEIGSKGKIEGISEERIKWLEGLISRYEEMVNLKSFVLPGGTLESAKLDVCRTIARRAERKVATVLREFGIGKEALVYLNRLSDLLFLLARVIEIEGS

SEQ ID NO: 221 - protein, 119 aa, Artificial Sequence
  Synthetic
GSMKKVEAIIRPEKLEIVKKALSDAGYVGMTVSEVKGRGVQGGIVERYRGREYIVDLIPKVKIELVVKQADVDNVIDIICENARTGNPGDGKIFVIPVQRVVRVRTKEEGAAALLEHGS

SEQ ID NO: 222 - protein, 101 aa, Artificial Sequence
  Synthetic
ILVYSTFPNEEKALEIGRKLLEKRLIACFNAFEIRSGYWWKGEIVQDKEWAAIFKTTEEKEKELYEELRKLHPYETPAIFTLKVENVLTEYMNWLRESVGG

SEQ ID NO: 223 - protein, 117 aa, Artificial Sequence
  Synthetic
GSMKKVEAIIRPEKLEIVKKALSDAGYVGMTVSEVKGRGVQGGIVERYRGREYIVDLIPKVKIELVVKEEDVDNVIDIICENARTGNPGDGKIFVIPVERVVRVRTKEEGKEALSNS

SEQ ID NO: 224 - protein, 109 aa, Artificial Sequence
  Synthetic
KKIEAIIRPFKLDEVKIALVNAGIVGMTVSEVRGFGRQKRGSEYTVEFLQKLKLEIVVEDAQVDTVIDKIVAAARTGENGDGKIFVSPVDQTIRIRTGEKNADAIGGGS

SEQ ID NO: 225 - protein, 100 aa, Artificial Sequence
  Synthetic
HLTPREFDKLVIHMLSDVALKRKNKGLKLNHPEAVAVLSAYVLDGAREGKTVEEVMDGARSVLKADDVMDGVPDLLPLIQVEAVFSDGSRLVSLHNPING

SEQ ID NO: 226 - protein, 104 aa, Artificial Sequence
  Synthetic
MEEVVLITVPSEEVARTIAKALVEERLAACVNIVPGLTSIYRWQGEVVEDQELLLLVKTTTHAFPKLKERVKALHPYTVPEIVALPIAQGNQEYLDWLRENAGS

SEQ ID NO: 227 - protein, 102 aa, Artificial Sequence
  Synthetic
MEEVVLITVPSEEVARTIAKALVEERLAACVNIVPGLTSIYRWQGEVVEDQELLLLVKTTTHAFPKLKERVKALHPYTVPEIVALPIAQGNQEYLDWLRENA

SEQ ID NO: 228 - protein, 87 aa, Artificial Sequence
  Synthetic
GIVNVPNCNTTKYQQLARTAVAIYNYHEQAHLTFVENLNCKEQLGEGDYYYITLAATDDAGKKLIYEAKIGVVESAGWTGVEEFKLV

SEQ ID NO: 229 - protein, 87 aa, Artificial Sequence
  Synthetic
GIVNVPNCNTTKYQQLARTAVAIYNYHEEAHLTFVENLNCKEQLGEGDYYYITLAATDDAGKKAIYEAKIGVVESAGWTGVEEFKLV

SEQ ID NO: 230 - protein, 87 aa, Artificial Sequence
  Synthetic
GIVNVPNCNTTKYQQLARTAVAIYNYHYQAHLTFVENLNCKEQLGEGDYYYITLAATDDAGKKAIYEAKIGVVESAGWTGVEEFKLV

SEQ ID NO: 231 - protein, 87 aa, Artificial Sequence
  Synthetic
GIVNVPNCNTTKYQRLARTAVAIYNYHEQAHLTFVENLNCKEQLGEGDYYYITLAATDDAGKKAIYEAKIGVVESAGWTGVEEFKLV

SEQ ID NO: 232 - protein, 87 aa, Artificial Sequence
  Synthetic
GIVNVPNCNTTKYQQLARTAVAIYNYHEQAHLQFVENLNCKEQLGEGDYYYITLAATDDAGKKAIYEAKIGVVESAGWTGVEEFKLV

SEQ ID NO: 233 - protein, 87 aa, Artificial Sequence
  Synthetic
GIVNVPNCNTTKYQQLARTAVAIYNYHEQAHLRFVENLNCKEQLGEGDYYYITLAATDDAGKKAIYEAKIGVVESAGWTGVEEFKLV

SEQ ID NO: 234 - protein, 87 aa, Artificial Sequence; identical to SEQ ID NO: 135
  Synthetic

SEQ ID NO: 235 - protein, 87 aa, Artificial Sequence
  Synthetic
GIVNVPNCNTTKYQQLARTAVAIYNYHEQAHLTFVENLNCKEQLGEGDYYYITLAATDDAGKKAIYEAKIGVVESSGWTGVEEFKLV

SEQ ID NO: 236 - protein, 87 aa, Artificial Sequence
  Synthetic
GIVNVPNCNTTKYQQLARTAVAIYNYHEQAHLTFVENLNCKEQLGEGDWYYITLAATDDAGKKAIYEAKIGVVESAGWTGVEEFKLV

SEQ ID NO: 237 - protein, 87 aa, Artificial Sequence; identical to SEQ ID NO: 135
  Synthetic

SEQ ID NO: 238 - protein, 87 aa, Artificial Sequence
  Synthetic
GIVNVPNCNTTKYQQLARTAVHIYNYHEQAHLTFVENLNCKEQLGEGDYYYITLAATDDAGKKAIYEAKIGVVESAGWTGVEEFKLV

SEQ ID NO: 239 - protein, 87 aa, Artificial Sequence
  Synthetic
GIVNVPNCNTTKYQQLARTAVAIYNYHEQAHLKFVENLNCKEQLGEGDYYYITLAATDDAGKKAIYEAKIGVVESAGWTGVEEFKLV

SEQ ID NO: 240 - protein, 87 aa, Artificial Sequence
  Synthetic
GIVNVPNCNTTKYQQLGRTAVAIYNYHEQAHLTFVENLNCKEQLGEGDYYYITLAATDDAGKKAIYEAKIGVVESAGWTGVEEFKLV

SEQ ID NO: 241 - protein, 87 aa, Artificial Sequence; identical to SEQ ID NO: 135
  Synthetic

SEQ ID NO: 242 - protein, 87 aa, Artificial Sequence; identical to SEQ ID NO: 135
  Synthetic

SEQ ID NO: 243 - protein, 87 aa, Artificial Sequence
  Synthetic
GIVNVPNCNTTKYQDLAQTAVAIYNYHEQAHLTFVENLNCKEQINGYDYYYITLAATDDAGKKAIYEAKIGVVESAGWFGVEEFKLV

SEQ ID NO: 244 - protein, 87 aa, Artificial Sequence
  Synthetic
GIVNVPNCNTTKYQQLAQTAVAIYNYHEQAHLTFVENLNCKEQLGEGDYYYITLAATDDAGKKAIYEAKIGVVESAGWTGVEEFKLV

SEQ ID NO: 245 - protein, 87 aa, Artificial Sequence
  Synthetic
GIVNVPNCNTTKYQQLAQTAVAIYNYHEQAHLTFVENLNCKEQLGEGDRYYITLAATDDAGKKAIYEAKIGVVESAGWTGVEEFKLV

SEQ ID NO: 246 - protein, 87 aa, Artificial Sequence
  Synthetic
GIVNVPNCNTTKYQQLAQTAVAIYNYHEQAHLTFVENLNCKEQLGGPDRYYITLAATDDAGKKAIYEAKIGVVEPAGWVAVMEFKLV

SEQ ID NO: 247 - protein, 87 aa, Artificial Sequence
  Synthetic
GIVNVPNCNTTKYQDLAQTAVAIYNYHEQAHLTFVENLNCKEQLGEGDYYYITLAATDDAGKKAIYEAKIGVVESAGWKGVEEFKLV

SEQ ID NO: 248 - protein, 87 aa, Artificial Sequence
  Synthetic
GIVNVPNCNTTKYQQLARTAVAIYNYHEQAHLTFVENLNCKEQLGGPDRYYITLAATDDAGKKAIYEAKIGVVEPAGWVAVEEFKLV

SEQ ID NO: 249 - protein, 87 aa, Artificial Sequence
  Synthetic
GIVNVPNCNTTKYQQLARSAVAIYNYHEQAHLTFVENLNCKEQLNDGIYYYITLAATDDAGKKAIYEAKIGVVESAGWIGVEEFKLV

SEQ ID NO: 250 - protein, 87 aa, Artificial Sequence
  Synthetic
GIVNVPNCNTTKYQRLADRAVAIYNYHEQAHLTFVENLNCKEQVGEGVYYYITLAATDDAGKKAIYEAKIGVVEPAGWVAVEEFKLV

SEQ ID NO: 251 - protein, 87 aa, Artificial Sequence
  Synthetic
GIVNVPNCNTTKYQQLAQRAVAIYNYHEQAHLTFVENLNCKEQWGLGIRYYITLAATDDAGKKAIYEAKIGVVEPAGWVAVMEFKLV

SEQ ID NO: 252 - protein, 87 aa, Artificial Sequence
  Synthetic
GIVNVPNCNTTKYQQLARTAVAIYNYHEQAHLTFVENLNCKEQLGGPDRYYITLAATDDAGKKAIYEAKIGVVEPAGWVAVMEFKLV

SEQ ID NO: 253 - protein, 87 aa, Artificial Sequence
  Synthetic
GIVNVPNCNTTKYQQLAQRAVAIYNYHEQAHLTFVENLNCKEQINGYDYYYITLAATDDAGKKAIYEAKIGVVESAGWTGVEEFKLV

SEQ ID NO: 254 - protein, 87 aa, Artificial Sequence
  Synthetic
GIVNVPNCNTTKYQQLAQRAVAIYNYHEQAHLTFVENLNCKEQWGLGIMYYITLAATDDAGKKAIYEAKIGVVEPAGWVAVMEFKLV

SEQ ID NO: 255 - protein, 87 aa, Artificial Sequence
  Synthetic
GIVNVPNCNTTKYQQLAQTAVAIYNYHEQAHLTFVENLNCKEQWGLGIMYYITLAATDDAGKKAIYEAKIGVVEPAGWVAVEEFKLV

SEQ ID NO: 256 - protein, 87 aa, Artificial Sequence
  Synthetic
GIVNVPNCNTTKYQQLAQRAVAIYNYHEQAHLTFVENLNCKEQWGLGIMYYITLAATDDAGKKAIYEAKIGVVEPAGWVAVEEFKLV

SEQ ID NO: 257 - protein, 87 aa, Artificial Sequence
  Synthetic
GIVNVPNCNTTKYQQLAQTAVAIYNYHEQAHLTFVENLNCKEQWGLGIMYYITLAATDDAGKKAIYEAKIGVVEPAGWVAVMEFKLV

SEQ ID NO: 258 - protein, 87 aa, Artificial Sequence
  Synthetic
GIVNVPNCNTTKYQDLAQTAVAIYNYHEQAHLTFVENLNCKEQLGEGDYYYITLAATDDAGKKAIYEAKIGVVESAGWFGVEEFKLV

SEQ ID NO: 259 - protein, 87 aa, Artificial Sequence
  Synthetic
GIVNVPNCNTTKYQQLAQDAVAIYNYHEQAHLTFVENLNCKEQLGEGDRYYITLAATDDAGKKAIYEAKIGVVESAGWTGVEEFKLV

SEQ ID NO: 260 - protein, 87 aa, Artificial Sequence
  Synthetic
GIVNVPNCNTTKYQDLAQTAVAIYNYHEQAHLTFVENLNCKEQLGDGVYYYITLAATDDAGKKAIYEAKIGVVESAGWKGVEEFKLV

SEQ ID NO: 261 - protein, 87 aa, Artificial Sequence
  Synthetic
GIVNVPNCNTTKYQQLARSAVAIYNYHEQAHLTFVENLNCKEQVSYGAYYYITLAATDDAGKKAIYEAKIGVVESAGWVGVEEFKLV

SEQ ID NO: 262 - protein, 87 aa, Artificial Sequence
  Synthetic
GIVNVPNCNTTKYQQLARSAVAIYNYHEQAHLTFVENLNCKEQTAKGNYYYITLAATDDAGKKAIYEAKIGVVESAGWVGVEEFKLV

SEQ ID NO: 263 - protein, 87 aa, Artificial Sequence
  Synthetic
GIVNVPNCNTTKYQDLAQTAVAIYNYHEQAHLTFVENLNCKEQLGDGVYYYITLAATDDAGKKAIYEAKIGVVESAGWFGVEEFKLV

SEQ ID NO: 264 - protein, 87 aa, Artificial Sequence
  Synthetic
GIVNVPNCNTTKYQQLARSAVAIYNYHEQAHLTFVENLNCKEQAGLGGYYYITLAATDDAGKKAIYEAKIGVVESAGWVAVGEFKLV

SEQ ID NO: 265 - protein, 87 aa, Artificial Sequence
  Synthetic
GIVNVPNCNTTKYQQLAQRAVAIYNYHEQAHLTFVENLNCKEQLGEGDYYYITLAATDDAGKKAIYEAKIGVVESAGWTGVEEFKLV

SEQ ID NO: 266 - protein, 87 aa, Artificial Sequence
  Synthetic
GIVNVPNCNTTKYQQLARSAVAIYNYHEQAHLTFVENLNCKEQVGDGDYYYITLAATDDAGKKAIYEAKIGVVESAGWVGVNEFKLV

SEQ ID NO: 267 - protein, 87 aa, Artificial Sequence
  Synthetic
GIVNVPNCNTTKYQQLARSAVAIYNYHEQAHLTFVENLNCKEQVGGMNYYYITLAATDDAGKKAIYEAKIGVVESAGWKGVEEFKLV

SEQ ID NO: 268 - protein, 87 aa, Artificial Sequence
  Synthetic
GIVNVPNCNTTKYQQLAQRAVAIYNYHEQAHLTFVENLNCKEQLGGPDRYYITLAATDDAGKKAIYEAKIGVVEPAGWVAVEEFKLV

SEQ ID NO: 269 - protein, 87 aa, Artificial Sequence
  Synthetic
GIVNVPNCNTTKYQQLARTAVAIYNYHEQAHLTFVENLNCKEQLGDGVYYYITLAATDDAGKKAIYEAKIGVVESAGWTGVEEFKLV

SEQ ID NO: 270 - protein, 87 aa, Artificial Sequence
  Synthetic
GIVNVPNCNTTKYQQLAQTAVAIYNYHEQAHLTFVENLNCKEQWGLGIRYYITLAATDDAGKKAIYEAKIGVVEPAGWVAVEEFKLV

SEQ ID NO: 271 - protein, 87 aa, Artificial Sequence
  Synthetic
GIVNVPNCNTTKYQQLARRAVAIYNYHEQAHLTFVENLNCKEQAGIGGYYYITLAATDDAGKKAIYEAKIGVVESAGWVAVGEFKLV

SEQ ID NO: 272 - protein, 87 aa, Artificial Sequence
  Synthetic
GIVNVPNCNTTKYQQLARIAVAIYNYHEQAHLTFVENLNCKEQIGEGNYYYITLAATDDAGKKAIYEAKIGVVESAGWIGVEEFKLV

SEQ ID NO: 273 - protein, 87 aa, Artificial Sequence
  Synthetic
GIVNVPNCNTTKYQRLADRAVAIYNYHEQAHLTFVENLNCKEQLGEGDRYYITLAATDDAGKKAIYEAKIGVVEPAGWVAVEEFKLV

SEQ ID NO: 274 - protein, 87 aa, Artificial Sequence
  Synthetic
GIVNVPNCNTTKYQQLAQRAVAIYNYHEQAHLTFVENLNCKEQLGDGVYYYITLAATDDAGKKAIYEAKIGVVESAGWTGVEEFKLV

SEQ ID NO: 275 - protein, 87 aa, Artificial Sequence
  Synthetic
GIVNVPNCNTTKYQQLARTAVAIYNYHEQAHLTFVENLNCKEQLTGYDYYYITLAATDDAGKKAIYEAKIGVVESAGWTGVEEFKLV

SEQ ID NO: 276 - protein, 87 aa, Artificial Sequence
  Synthetic
GIVNVPNCNTTKYQDLAQTAVAIYNYHEQAHLTFVENLNCKEQLTGYDYYYITLAATDDAGKKAIYEAKIGVVESAGWKGVEEFKLV

SEQ ID NO: 277 - protein, 87 aa, Artificial Sequence
  Synthetic
GIVNVPNCNTTKYQQLAQRAVAIYNYHEQAHLTFVENLNCKEQLTGYDYYYITLAATDDAGKKAIYEAKIGVVESAGWTGVEEFKLV

SEQ ID NO: 278 - protein, 87 aa, Artificial Sequence
  Synthetic
GIVNVPNCNTTKYQQLARRAVAIYNYHEQAHLTFVENLNCKEQINGYDYYYITLAATDDAGKKAIYEAKIGVVESAGWEVVGEFKLV

SEQ ID NO: 279 - protein, 87 aa, Artificial Sequence
  Synthetic
GIVNVPNCNTTKYQQLAQTAVAIYNYHEQAHLTFVENLNCKEQLGGPDRYYITLAATDDAGKKAIYEAKIGVVEPAGWVAVEEFKLV

SEQ ID NO: 280 - protein, 87 aa, Artificial Sequence
  Synthetic
GIVNVPNCNTTKYQQLAQRAVAIYNYHEQAHLTFVENLNCKEQVGEGVYYYITLAATDDAGKKAIYEAKIGVVESAGWTGVEEFKLV

SEQ ID NO: 281 - protein, 87 aa, Artificial Sequence
  Synthetic
GIVNVPNCNTTKYQRLADRAVAIYNYHEQAHLTFVENLNCKEQLGDGVYYYITLAATDDAGKKAIYEAKIGVVEPAGWVAVEEFKLV

SEQ ID NO: 282 - protein, 87 aa, Artificial Sequence
  Synthetic
GIVNVPNCNTTKYQQLAQRAVAIYNYHEQAHLTFVENLNCKEQLGGPDRYYITLAATDDAGKKAIYEAKIGVVEPAGWVAVMEFKLV

SEQ ID NO: 283 - protein, 87 aa, Artificial Sequence
  Synthetic
GIVNVPNCNTTKYQQLAQDAVAIYNYHEQAHLTFVENLNCKEQWGLGIMYYITLAATDDAGKKAIYEAKIGVVEPAGWVAVEEFKLV

SEQ ID NO: 284 - protein, 87 aa, Artificial Sequence
  Synthetic
GIVNVPNCNTTKYQQLARIAVAIYNYHEQAHLTFVENLNCKEQVSGYDYYYITLAATDDAGKKAIYEAKIGVVESAGWTGVEEFKLV

SEQ ID NO: 285 - protein, 87 aa, Artificial Sequence
  Synthetic
GIVNVPNCNTTKYQRLADRAVAIYNYHEQAHLTFVENLNCKEQLGEGDRYYITLAATDDAGKKAIYEAKIGVVESAGWTGVEEFKLV

SEQ ID NO: 286 - protein, 87 aa, Artificial Sequence
  Synthetic
GIVNVPNCNTTKYQQLAQDAVAIYNYHEQAHLTFVENLNCKEQLGGPDRYYITLAATDDAGKKAIYEAKIGVVEPAGWVAVMEFKLV

SEQ ID NO: 287 - protein, 87 aa, Artificial Sequence
  Synthetic
GIVNVPNCNTTKYQQLAQRAVAIYNYHEQAHLTFVENLNCKEQLGEGDRYYITLAATDDAGKKAIYEAKIGVVESAGWTGVEEFKLV

SEQ ID NO: 288 - protein, 87 aa, Artificial Sequence
  Synthetic
GIVNVPNCNTTKYQRLADRAVAIYNYHEQAHLTFVENLNCKEQVGEGVYYYITLAATDDAGKKAIYEAKIGVVESAGWTGVEEFKLV

SEQ ID NO: 289 - protein, 87 aa, Artificial Sequence
  Synthetic
GIVNVPNCNTTKYQQLARSAVAIYNYHEQAHLTFVENLNCKEQIDYGAYYYITLAATDDAGKKAIYEAKIGVVESAGWIGVEEFKLV

SEQ ID NO: 290 - protein, 87 aa, Artificial Sequence
  Synthetic
GIVNVPNCNTTKYQQLARIAVAIYNYHEQAHLTFVENLNCKEQLAVGDYYYITLAATDDAGKKAIYEAKIGVVESAGWIGVEEFKLV

SEQ ID NO: 291 - protein, 87 aa, Artificial Sequence
  Synthetic
GIVNVPNCNTTKYQQLAQTAVAIYNYHEQAHLTFVENLNCKEQWGLGIRYYITLAATDDAGKKAIYEAKIGVVEPAGWVAVMEFKLV

SEQ ID NO: 292 - protein, 87 aa, Artificial Sequence
  Synthetic
GIVNVPNCNTTKYQQLARSAVAIYNYHEQAHLTFVENLNCKEQIAGKTYYYITLAATDDAGKKAIYEAKIGVVESAGWEGVEEFKLV

SEQ ID NO: 293 - protein, 87 aa, Artificial Sequence
  Synthetic
GIVNVPNCNTTKYQQLARSAVAIYNYHEQAHLTFVENLNCKEQSSGYSYYYITLAATDDAGKKAIYEAKIGVVESAGWEAVTEFKLV

SEQ ID NO: 294 - protein, 87 aa, Artificial Sequence
  Synthetic
GIVNVPNCNTTKYQQLARTAVAIYNYHEQAHLTFVENLNCKEQWGLGIRYYITLAATDDAGKKAIYEAKIGVVEPAGWVAVEEFKLV

SEQ ID NO: 295 - protein, 87 aa, Artificial Sequence
  Synthetic
GIVNVPNCNTTKYQQLAQRAVAIYNYHEQAHLTFVENLNCKEQWGLGIRYYITLAATDDAGKKAIYEAKIGVVEPAGWVAVEEFKLV

SEQ ID NO: 296 - protein, 87 aa, Artificial Sequence
  Synthetic
GIVNVPNCNTTKYQRLADRAVAIYNYHEQAHLTFVENLNCKEQLTGYDYYYITLAATDDAGKKAIYEAKIGVVESAGWTGVEEFKLV

SEQ ID NO: 297 - protein, 87 aa, Artificial Sequence
  Synthetic
GIVNVPNCNTTKYQQLARSAVAIYNYHEQAHLTFVENLNCKEQLTGFDYYYITLAATDDAGKKAIYEAKIGVVESAGWEGVNEFKLV

SEQ ID NO: 298 - protein, 87 aa, Artificial Sequence
  Synthetic
GIVNVPNCNTTKYQQLARSAVAIYNYHEQAHLTFVENLNCKEQLGGYSYYYITLAATDDAGKKAIYEAKIGVVESAGWVGVGEFKLV

SEQ ID NO: 299 - protein, 87 aa, Artificial Sequence
  Synthetic
GIVNVPNCNTTKYQQLARSAVAIYNYHEQAHLTFVENLNCKEQINDGVYYYITLAATDDAGKKAIYEAKIGVVESAGWVGVEEFKLV

SEQ ID NO: 300 - protein, 87 aa, Artificial Sequence
  Synthetic
GIVNVPNCNTTKYQQLARSAVAIYNYHEQAHLTFVENLNCKEQSDLGSYYYITLAATDDAGKKAIYEAKIGVVESAGWIGVEEFKLV

SEQ ID NO: 301 - protein, 87 aa, Artificial Sequence
  Synthetic
GIVNVPNCNTTKYQQLARRAVAIYNYHEQAHLTFVENLNCKEQTGLGIYYYITLAATDDAGKKAIYEAKIGVVESAGWVAVGEFKLV

SEQ ID NO: 302 - protein, 87 aa, Artificial Sequence
  Synthetic
GIVNVPNCNTTKYQDLAQTAVAIYNYHEQAHLTFVENLNCKEQINGYDYYYITLAATDDAGKKAIYEAKIGVVESAGWKGVEEFKLV

SEQ ID NO: 303 - protein, 87 aa, Artificial Sequence
  Synthetic
GIVNVPNCNTTKYQQLARSAVAIYNYHEQAHLTFVENLNCKEQTGGPAYYYITLAATDDAGKKAIYEAKIGVVESAGWIGVEEFKLV

SEQ ID NO: 304 - protein, 87 aa, Artificial Sequence
  Synthetic
GIVNVPNCNTTKYQQLAQDAVAIYNYHEQAHLTFVENLNCKEQWGLGIMYYITLAATDDAGKKAIYEAKIGVVEPAGWVAVMEFKLV

SEQ ID NO: 305 - protein, 87 aa, Artificial Sequence
  Synthetic
GIVNVPNCNTTKYQQLAQDAVAIYNYHEQAHLTFVENLNCKEQVGEGVYYYITLAATDDAGKKAIYEAKIGVVESAGWTGVEEFKLV

SEQ ID NO: 306 - protein, 87 aa, Artificial Sequence
  Synthetic
GIVNVPNCNTTKYQQLARTAVAIYNYHEQAHLTFVENLNCKEQINGYDYYYITLAATDDAGKKAIYEAKIGVVESAGWTGVEEFKLV

SEQ ID NO: 307 - protein, 87 aa, Artificial Sequence
  Synthetic
GIVNVPNCNTTKYQQLARIAVAIYNYHEQAHLTFVENLNCKEQLGSGTYYYITLAATDDAGKKAIYEAKIGVVESAGWTGVEEFKLV

SEQ ID NO: 308 - protein, 87 aa, Artificial Sequence
  Synthetic
GIVNVPNCNTTKYQRLADRAVAIYNYHEQAHLTFVENLNCKEQINGYDYYYITLAATDDAGKKAIYEAKIGVVEPAGWVAVEEFKLV

SEQ ID NO: 309 - protein, 87 aa, Artificial Sequence
  Synthetic
GIVNVPNCNTTKYQQLAQTAVAIYNYHEQAHLTFVENLNCKEQLTGYDYYYITLAATDDAGKKAIYEAKIGVVESAGWTGVEEFKLV

SEQ ID NO: 310 - protein, 87 aa, Artificial Sequence
  Synthetic
GIVNVPNCNTTKYQQLAQTAVAIYNYHEQAHLTFVENLNCKEQVGEGVYYYITLAATDDAGKKAIYEAKIGVVESAGWTGVEEFKLV

SEQ ID NO: 311 - protein, 87 aa, Artificial Sequence
  Synthetic
GIVNVPNCNTTKYQQLARTAVAIYNYHEQAHLTFVENLNCKEQWGLGIMYYITLAATDDAGKKAIYEAKIGVVEPAGWVAVEEFKLV

SEQ ID NO: 312 - protein, 87 aa, Artificial Sequence
  Synthetic
GIVNVPNCNTTKYQQLAQTAVAIYNYHEQAHLTFVENLNCKEQLGDGVYYYITLAATDDAGKKAIYEAKIGVVESAGWTGVEEFKLV

SEQ ID NO: 313 - protein, 87 aa, Artificial Sequence
  Synthetic
GIVNVPNCNTTKYQQLAQDAVAIYNYHEQAHLTFVENLNCKEQLTGYDYYYITLAATDDAGKKAIYEAKIGVVESAGWTGVEEFKLV

SEQ ID NO: 314 - protein, 87 aa, Artificial Sequence; identical to SEQ ID NO: 135
  Synthetic

SEQ ID NO: 315 - protein, 87 aa, Artificial Sequence
  Synthetic
GIVNVPNCNTTKYQQLARTAVAIYNYHEQAHLTFVENLNCKEQWGLGIRYYITLAATDDAGKKAIYEAKIGVVEPAGWVAVMEFKLV

SEQ ID NO: 316 - protein, 87 aa, Artificial Sequence
  Synthetic
GIVNVPNCNTTKYQQLAQDAVAIYNYHEQAHLTFVENLNCKEQINGYDYYYITLAATDDAGKKAIYEAKIGVVESAGWTGVEEFKLV

SEQ ID NO: 317 - protein, 87 aa, Artificial Sequence
  Synthetic
GIVNVPNCNTTKYQRLADRAVAIYNYHEQAHLTFVENLNCKEQLGDGVYYYITLAATDDAGKKAIYEAKIGVVESAGWTGVEEFKLV

SEQ ID NO: 318 - protein, 87 aa, Artificial Sequence
  Synthetic
GIVNVPNCNTTKYQQLARTAVAIYNYHEQAHLTFVENLNCKEQVGEGVYYYITLAATDDAGKKAIYEAKIGVVESAGWTGVEEFKLV

SEQ ID NO: 319 - protein, 87 aa, Artificial Sequence
  Synthetic
GIVNVPNCNTTKYQQLAQDAVAIYNYHEQAHLTFVENLNCKEQLGEGDYYYITLAATDDAGKKAIYEAKIGVVESAGWTGVEEFKLV

SEQ ID NO: 320 - protein, 87 aa, Artificial Sequence
  Synthetic
GIVNVPNCNTTKYQQLARSAVAIYNYHEQAHLTFVENLNCKEQLGEGDYYYITLAATDDAGKKAIYEAKIGVVESAGWTGVEEFKLV

SEQ ID NO: 321 - protein, 87 aa, Artificial Sequence
  Synthetic
GIVNVPNCNTTKYQQLAQDAVAIYNYHEQAHLTFVENLNCKEQWGLGIRYYITLAATDDAGKKAIYEAKIGVVEPAGWVAVEEFKLV

SEQ ID NO: 322 - protein, 87 aa, Artificial Sequence
  Synthetic
GIVNVPNCNTTKYQDLAQTAVAIYNYHEQAHLTFVENLNCKEQLTGYDYYYITLAATDDAGKKAIYEAKIGVVESAGWFGVEEFKLV

SEQ ID NO: 323 - protein, 87 aa, Artificial Sequence
  Synthetic
GIVNVPNCNTTKYQRLADRAVAIYNYHEQAHLTFVENLNCKEQLTGYDYYYITLAATDDAGKKAIYEAKIGVVEPAGWVAVEEFKLV

SEQ ID NO: 324 - protein, 87 aa, Artificial Sequence
  Synthetic
GIVNVPNCNTTKYQQLARSAVAIYNYHEQAHLTFVENLNCKEQISEGIYYYITLAATDDAGKKAIYEAKIGVVESAGWIGVEEFKLV

SEQ ID NO: 325 - protein, 87 aa, Artificial Sequence
  Synthetic
GIVNVPNCNTTKYQQLAQDAVAIYNYHEQAHLTFVENLNCKEQLGGPDRYYITLAATDDAGKKAIYEAKIGVVEPAGWVAVEEFKLV

SEQ ID NO: 326 - protein, 87 aa, Artificial Sequence
  Synthetic
GIVNVPNCNTTKYQQLARRAVAIYNYHEQAHLTFVENLNCKEQTNGDVYYYITLAATDDAGKKAIYEAKIGVVESAGWVGVSEFKLV

SEQ ID NO: 327 - protein, 87 aa, Artificial Sequence
  Synthetic
GIVNVPNCNTTKYQQLARTAVAIYNYHEQAHLTFVENLNCKEQLGEGDRYYITLAATDDAGKKAIYEAKIGVVESAGWTGVEEFKLV

SEQ ID NO: 328 - protein, 87 aa, Artificial Sequence
  Synthetic
GIVNVPNCNTTKYQQLAQTAVAIYNYHEQAHLTFVENLNCKEQINGYDYYYITLAATDDAGKKAIYEAKIGVVESAGWTGVEEFKLV

SEQ ID NO: 329 - protein, 87 aa, Artificial Sequence
  Synthetic
GIVNVPNCNTTKYQQLARTAVAIYNYHEQAHLTFVENLNCKEQWGLGIMYYITLAATDDAGKKAIYEAKIGVVEPAGWVAVMEFKLV

SEQ ID NO: 330 - protein, 87 aa, Artificial Sequence
  Synthetic
GIVNVPNCNTTKYQDLAQTAVAIYNYHEQAHLTFVENLNCKEQVGEGVYYYITLAATDDAGKKAIYEAKIGVVESAGWFGVEEFKLV

SEQ ID NO: 331 - protein, 87 aa, Artificial Sequence
  Synthetic
GIVNVPNCNTTKYQDLAQTAVAIYNYHEQAHLTFVENLNCKEQLGEGDRYYITLAATDDAGKKAIYEAKIGVVESAGWKGVEEFKLV

SEQ ID NO: 332 - protein, 87 aa, Artificial Sequence
  Synthetic
GIVNVPNCNTTKYQQLARRAVAIYNYHEQAHLTFVENLNCKEQTGFGNYYYITLAATDDAGKKAIYEAKIGVVESAGWEGVSEFKLV

SEQ ID NO: 333 - protein, 87 aa, Artificial Sequence
  Synthetic
GIVNVPNCNTTKYQQLAQDAVAIYNYHEQAHLTFVENLNCKEQWGLGIRYYITLAATDDAGKKAIYEAKIGVVEPAGWVAVMEFKLV

SEQ ID NO: 334 - protein, 87 aa, Artificial Sequence
  Synthetic
GIVNVPNCNTTKYQQLARRAVAIYNYHEQAHLTFVENLNCKEQSGVGVYYYITLAATDDAGKKAIYEAKIGVVESAGWVAVGEFKLV

SEQ ID NO: 335 - protein, 87 aa, Artificial Sequence
  Synthetic
GIVNVPNCNTTKYQQLARRAVAIYNYHEQAHLTFVENLNCKEQIGFGDYYYITLAATDDAGKKAIYEAKIGVVESAGWEGVSEFKLV

SEQ ID NO: 336 - protein, 87 aa, Artificial Sequence
  Synthetic
GIVNVPNCNTTKYQRLADRAVAIYNYHEQAHLTFVENLNCKEQLGEGDYYYITLAATDDAGKKAIYEAKIGVVESAGWTGVEEFKLV

SEQ ID NO: 337 - protein, 87 aa, Artificial Sequence
  Synthetic
GIVNVPNCNTTKYQQLARIAVAIYNYHEQAHLTFVENLNCKEQSGLGDYYYITLAATDDAGKKAIYEAKIGVVESAGWTGVEEFKLV

SEQ ID NO: 338 - protein, 87 aa, Artificial Sequence
  Synthetic
GIVNVPNCNTTKYQQLARSAVAIYNYHEQAHLTFVENLNCKEQIGDGTYYYITLAATDDAGKKAIYEAKIGVVESAGWEVVSEFKLV

SEQ ID NO: 339 - protein, 87 aa, Artificial Sequence
  Synthetic
GIVNVPNCNTTKYQDLAQTAVAIYNYHEQAHLTFVENLNCKEQVGEGVYYYITLAATDDAGKKAIYEAKIGVVESAGWKGVEEFKLV

SEQ ID NO: 340 - protein, 87 aa, Artificial Sequence
  Synthetic
GIVNVPNCNTTKYQRLADRAVAIYNYHEQAHLTFVENLNCKEQLGEGDYYYITLAATDDAGKKAIYEAKIGVVEPAGWVAVEEFKLV

SEQ ID NO: 341 - protein, 87 aa, Artificial Sequence
  Synthetic
GIVNVPNCNTTKYQQLARRAVAIYNYHEQAHLTFVENLNCKEQTAGMGYYYITLAATDDAGKKAIYEAKIGVVESAGWVAVGEFKLV

SEQ ID NO: 342 - protein, 87 aa, Artificial Sequence
  Synthetic
GIVNVPNCNTTKYQQLARIAVAIYNYHEQAHLTFVENLNCKEQISDGVYYYITLAATDDAGKKAIYEAKIGVVESAGWTGVEEFKLV

SEQ ID NO: 343 - protein, 87 aa, Artificial Sequence
  Synthetic
GIVNVPNCNTTKYQQLAQDAVAIYNYHEQAHLTFVENLNCKEQLGDGVYYYITLAATDDAGKKAIYEAKIGVVESAGWTGVEEFKLV

SEQ ID NO: 344 - protein, 87 aa, Artificial Sequence
  Synthetic
GIVNVPNCNTTKYQDLAQTAVAIYNYHEQAHLTFVENLNCKEQLGEGDRYYITLAATDDAGKKAIYEAKIGVVESAGWFGVEEFKLV

SEQ ID NO: 345 - protein, 87 aa, Artificial Sequence
  Synthetic
GIVNVPNCNTTKYQQLARRAVAIYNYHEQAHLTFVENLNCKEQLTGYDYYYITLAATDDAGKKAIYEAKIGVVESAGWEVVAEFKLV

SEQ ID NO: 346 - protein, 87 aa, Artificial Sequence
  Synthetic
GIVNVPNCNTTKYQRLADRAVAIYNYHEQAHLTFVENLNCKEQINGYDYYYITLAATDDAGKKAIYEAKIGVVESAGWTGVEEFKLV

SEQ ID NO: 347 - protein, 5 aa, Artificial Sequence
  Synthetic
CYIGN

SEQ ID NO: 348 - protein, 5 aa, Artificial Sequence
  Synthetic
AGSQQ

SEQ ID NO: 349 - protein, 5 aa, Artificial Sequence
  Synthetic
AGEEN

SEQ ID NO: 350 - protein, 6 aa, Artificial Sequence
  Synthetic
AYSDQK

SEQ ID NO: 351 - protein, 5 aa, Artificial Sequence
  Synthetic
TGGGS

SEQ ID NO: 352 - protein, 8 aa, Artificial Sequence
  Synthetic
GGGGSGGS

SEQ ID NO: 353 - protein, 4 aa, Artificial Sequence
  Synthetic
GGGS

(19) United States
(12) Patent Application Publication
(10) Pub. No.: US 2017/0198014 A1
(43) Pub. Date: Jul. 13, 2017

(54) POLYPEPTIDES AND THEIR USE FOR TREATING INFLUENZA

(71) Applicant: UNIVERSITY WASHINGTON THROUGH ITS CENTER FOR COMMERCIALIZATION, Seattle, WA (US)

(72) Inventors: Eva-Maria STRAUCH, Seattle, WA (US); William H. SHEFFLER, Seattle, WA (US); David BAKER, Seattle, WA (US)

(21) Appl. No.: 15/320,936

(22) PCT Filed: Aug. 26, 2015
(86) PCT No.: PCT/US15/46920

§ 371 Date: Dec. 21, 2016

Related U.S. Application Data
(60) Provisional application No. 62/041,981, filed on Aug. 26, 2014.

(51) Int. Cl. C07K 14/00

(52) U.S. Cl. CPC C07K 14/00; A61K 38/00

(57) ABSTRACT

Provided herein are scaffold polypeptides, fusion proteins of the scaffold polypeptides and hemagglutinin (HA) binding proteins, and methods for using the fusion proteins for treating and diagnosing influenza infections.

### CROSS REFERENCE

This application claims priority to U.S. Provisional Patent Application Ser. No. 62/041981 filed Aug. 26, 2014, incorporated by reference herein in its entirety.

### FEDERAL FUNDING STATEMENT

This invention was made with government support under N00014-10-D-6318/0024 awarded by Defense Threat Reduction Agency. The government has certain rights in the invention.

### BACKGROUND

Influenza virus is a member of Orthomyxoviridae family. There are three subtypes of influenza viruses designated A, B, and C. The influenza virion contains a segmented negative-sense RNA genome, encoding, among other proteins, henagglutinin (HA) and neuraminidase (NA). Influenza virus infection is initiated by the attachment of the virion surface HA protein to a sialic acid-containing cellular receptor (glycoproteins and glycolipids). The NA protein mediates processing of the sialic acid receptor, and virus penetration into the cell depends on HA-dependent receptor-mediated endocytosis. In the acidic confines of internalized endosomes containing an influenza virion, the HA2 protein undergoes conformational changes that lead to fusion of viral and cell membranes and virus uncoating and M2-mediated release of proteins from nucleocapsid-associated ribonucleoproteins (RNPs), which migrate into the cell nucleus for viral RNA synthesis. Its surface protein hemagglutinin (HA) attaches to the sialic acid moieties on the host cell surface and mediates entry into the cell. So far, chemical analogs of the receptor have not been successful as viral-entry blockers. Current treatment options include therapeutic antibodies, small-molecules drugs and vaccination. These therapies allow protection against circulating subtypes, but may not protect against newly emerging strains. Hence, general or quickly adaptable solutions for cheap treatment options represent constant need. Additionally, in order to rapidly diagnose early whether a patient indeed suffers from Influenza, sensitive diagnostics are desirable, as treatment at the onset of the infection have been shown to be more efficient.

Influenza presents a serious public-health challenge and new therapies are needed to combat viruses that are resistant to existing antivirals or escape neutralization by the immune system.

### SUMMARY OF THE INVENTION

In a first aspect, the invention provides isolated polypeptides comprising an amino acid sequence at least 90% identical over the full length of a polypeptide selected from the group consisting of SEQ ID NOS: 200-212 and SEQ ID NO:227. In one embodiment, the isolated polypeptide further comprises an amino acid linker sequence al its C-terminus. In one embodiment, the amino acid linker is between 1 amino acid and 20 amino acids in length; in another embodiment, the amino acid linker comprises an amino acid sequence selected from the group consisting of GS, GG, SNS, NG, and SEQ ID NOS: 347-353. In another embodiment, the invention provides multimers of the polypeptides of the invention, including but not limited to trimers, in one embodiment, the two or more monomeric units of the polypeptide in a multimer are identical. In another embodiment, the isolated polypeptide, or multimers thereof, further comprise a polypeptide receptor binding site (RBS) inhibitor selected from the group consisting of:

(a) a polypeptide at least 70% identical over the full length of the amino acid sequence of any one of SEQ ID NOS: 1-5; and

(b) a polypeptide comprising the amino acid sequence of any one of SEQ ID NOS: 6-97, 121-199, and 228-346.

In another aspect, the invention provides isolated polypeptides having the amino acid sequence of SEQ ID NO:1, wherein one or more of the following is true:

(a) 1, 2, or all 3 of amino acids 44-46 are absent;

(b) amino acid 11 is K or P;

(c) amino acid 14 is I or R;

(d) amino acid 15 is K or R;

(e) amino acid 17 is G;

(f) amino acid 19 is Q or T;

(g) amino acid 22 is H, S, or Y;

(h) amino acid 2.4 is I, M, V, or W.

(i) amino acid 27 is D;

(j) amino acid 28 is D, E, K, W, or

(k) amino acid 29 is D, E, or P;

(l) amino acid 30 is E;

(m) amino acid 33 is A, F, H, I, K, L, M, N, Q, R, S, T, or Y

(n) amino acid 36 is H or Q;

(o) amino acid 43 is K

(p) amino acid 47 is K;

(q) amino acid 49 is L, W, or Y;

(r) amino acid 51 is W;

(s) amino acid 59 is Q;

(t) amino acid 62 is E;

(u) amino acid 64 is A, G, L, M Q, S, or W;

(v) amino acid 67 is A, F, M, S, W, or Y;

(w) amino acid 68 is T;

(x) amino acid 70 is L;

(y) amino acid 76 is S;

(z) amino acid 79 is L;

(aa) amino acid 81 is L;

(bb) amino acid 85 is A, D, or E; and/or

(cc) amino acid 87 is 1 or M

In one embodiment, 1, 2, or all 3 of amino acids 44-46 are absent. In another embodiment, the isolated polypeptides comprise the amino acid sequence of a polypeptide selected from the group consisting of SEQ ID NOS: 77, 79, 135-199, and 228-346.

In one embodiment of any of the polypeptides of the invention, the polypeptides further comprise a tag, including but not limited to a detectable moiety and a therapeutic agent.

In another aspect, the invention provides a pharmaceutical composition comprising (i) the polypeptide, or multimers thereof, of any embodiment or combination of embodiments of the invention, and (2) a pharmaceutically acceptable carrier.

In another aspect, the invention provides isolated nucleic acids encoding the polypeptide of any embodiment or combination of embodiments of the invention. In another aspect, the invention provides recombinant expression vectors comprising a nucleic acid of the invention. In a further aspect, the invention provides recombinant host cells comprising a recombinant expression vector of the invention.

In another aspect, the invention provides an assembly, comprising:

(a) scaffold; and

(b) three hemagglutinin (HA) receptor binding site (RBS) inhibitors bound to the scaffold, wherein the scaffold organizes the three inhibitors such that each inhibitor is between about 40 A° and about 60 A° in distance from each other and at an angle of between about 57 degrees and about 63 degrees from each other. In one embodiment, each inhibitor is between about 41 A° and about 59 A°, about 42 A° and about 58 A°, about 43 A° and about 57 A°, about 44 A° and about 56 A°, about 45 A° and about 55 A°, about 46 A° and about 54 A°, about 47 A° and about 53 A°, about 48 A° and about 52 A°, about 49 A°and about 51 A°, or about 49.5 A° in distance from each other. In another embodiment, each inhibitor is about 49.5 A° in distance from each other. In a further embodiment, wherein each inhibitor is at an angle of between about 58 degrees and about 62 degrees from each other, about 59 degrees and about 61 degrees from each other, or about 60 degrees from each other, in another embodiment, each inhibitor is at an angle of about 60 degrees from each other. In another embodiment, the scaffold comprises a material selected from the group consisting of nucleic acids, polypeptides, organic molecules, inorganic molecules, lipids, carbohydrates, synthetic polymers, and combinations thereof. In a further embodiment, the scaffold comprises a polypeptide, such as a trimeric polypeptide of any embodiment or combination of embodiments of the present invention.

In another aspect, the invention provides methods for treating and/or limiting an influenza infection, comprising administering to a subject in need thereof a therapeutically effective amount of the polypeptide any embodiment or combination of embodiments of the present invention, the pharmaceutical composition of any embodiment or combination of embodiments of the present invention, or the assembly of any embodiment or combination of embodiments of the present invention, or salts thereof, to treat and/or limit the influenza infection.

In a further aspect, the invention provides methods for diagnosing an influenza infection, or monitoring progression of an influenza infection, comprising

(a) contacting a biological sample from a subject suspected of having an influenza infection with a diagnostically effective amount of the polypeptide according to any embodiment or combination of embodiments of the present invention, the pharmaceutical compositions of any embodiment or combination of embodiments of the present invention, or the assembly of any embodiment or combination of embodiments of the present invention, or salts thereof, under conditions suitable for binding of the polypeptide to a viral HA protein present in the sample;

(b) removing unbound polypeptide and/or sample; and

(c) detecting polypeptide-viral HA binding complexes,

where the presence of such binding complexes indicates that the subject has an influenza infection, or provides a measure of progression of an influenza infection.

In a still further aspect, the invention provides methods for identifying candidate compounds for treating, limiting, and/or diagnosing influenza infection, comprising

(a) contacting an influenza HA protein with (i) test compounds and (ii) the polypeptide according to any embodiment or combination of embodiments of the present invention, the pharmaceutical compositions of any embodiment or combination of embodiments of the present invention, or the assembly of any embodiment or combination of embodiments of the present invention, or salts thereof, under conditions suitable for binding of the HA protein to the polypeptide of the present invention; and

(b) identifying those test compounds that outcompete the polypeptide for binding to the HA protein, wherein such test compounds are candidate compounds for treating, limiting, and/or diagnosing influenza infection.

### DESCRIPTION OF THE FIGURES

FIG. 1. Model of 3 exemplary SB-52 variants bound to the head region of trimeric hemagglutinin. The distances between the end points of the binding protein that becomes the binding domain are 49.5 Angstrom which is bridged and connected through the self-assembling trimeric units

FIG. 2, Biolayer interferometry titrations. Association (A) and dissociation (D) of either monovalent SB52nc-2 (SEQ ID NO:96) or trimeric SB52nc-2 fused to HAA.1 with HA of the H3 A/Hong Kong/1/1968. Resulting binding constants are 229 nM and L5 nM respectively.

FIG. 3. Biolayer interferometry titrations. Association (A) and dissociation (D) of trimeric SB52nc-2 (SEQ fused to HAA.1 with HA of the H3 A/Victoria/361/2011.

FIG. 4. HAA.1c fused to Hsc1 via a triLong linker. Association (A) and dissociation (D) of monovalent versus trimeric form indicate the strong avidity effect that increases affinity more than 1000 fold. The monovalent version binds with a binding constant of 31.2 nM the Hong Kong H3 and 1120 nM the New Caledonia 1999 HA; whereas the trimeric version binds with a K_{D} below 1 nM (estimated of 0.06 pM).

FIG. 5. Binding of HAA.1 c fused to Hsc1-EA via a triLong linker to H3 Hong Kong resulting; in a binding constant below 1 nM,

### DETAILED DESCRIPTION OF THE INVENTION

All references cite(are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by a Goeddel, 1991. Academic Press, San Diego, Calif.), “Guide to Protein Purification” in Methods in Enzymology (M. P. Deutshcer, ed., (1990) Academic Press, Inc.): PCR Protocols: A Guide to Methods and Applications (Innis, et al. 1990. Academic Press, San Diego, Calif.), Culture of Animal Cells: A Manual of Basic Technique, 2^{nd}° Ed. (R. I. Freshney. 1987. Liss, Inc. New York, N.Y.), Gene Transfer and Expression Protocols, pp. 109-128, ed. E. J. Murray, The Humana Press Inc., Clifton, N. J.), and the Ambion 1998 Catalog (Ambion, Austin, Tex.).

As used herein, the singular forms “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used combination. unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used herein, the teen “about” means within +/−5% of the recited value.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize

In a first aspect, the invention provides isolated polypeptides comprising or consisting of an amino acid sequence at least 90% identical over their full length to a polypeptide selected from the group consisting of:

HAA.1

SEQ ID NO: 200

MEEVVLITVPSEEVARTIAKALVEERLAACVNIVPGLTSIYRWQGEVVED
QELLLLVKTTTHAFPKLKERVKALHPYTVPEIVALPIAQGNQEYLDWLRE
NT;
HAA.1.1

SEQ ID NO: 201

MEEVVLITVPSEEVARTIAKALVEERLAACVNIVPGLTSIYRWQGEVVED
QELLLLVKTTTAAFPKLKERVKALHPYTVPEIVALPIAEGNREYLDWLQE
HAA.2

SEQ ID NO: 202

ALYFSGHMILVYSTFPNEEKALEIGRKLLEKRLIACFNAFEIRSGYWWKG
EIVQDKEWAAIFKTTQSKQKELYEELRKLHPYETPAIFTLKVENILTEYM
NWLAESVLGS;
HAA.3

SEQ ID NO: 203

MKLIVAIVRPEKLNEVLKALFQAEVRGLTLSRVQGHGMELHEKVRLEIGV
SEPFVKPTVEAILKAARTGEVGDGKIFVLPVEKVYRIRTGEEA;
HAA.3.1

SEQ ID NO: 204

MKLIVAIVRPEKLNEVLKALFQAEVRGLTLSRVQGHGGETERVETYRGTT
VKMELHEKVRLEIGVSEPFVKPTVEAILKAARTGEVGDGKIFVLPVEKVY
RIRTGEEA;
HAA.4

SEQ ID NO: 205

KMEELFKKHKIVAVLRANSVEEAKEKALAVFEGGVHLIEITFTVPDADTV
IKELSFLKEKGAIIGAGTVTSVEQCRKAVESGAEFIVSPHLDEEISQFCK
EKGVFYMPGVMTPTELVKAMKLGHTILKLFPGQVVGPQFVKAMKGPFPNV
KFVPTGGVNLSNVAAWFAAGVLAVGVGSALVKGTPNNVREKAKAFVEAIR
GA;
HAA.5

SEQ ID NO: 206

KDSEIVKALGDLDELNSVLGVVSSLYPSLSQVIQKLQNDIFSISSEIAGF
DMNFSDEKVKGIEELITNYSKELEPLSNFVLPGGHIASSFLHLARAVCRR
AERSVVTLLKESKAKEVHAKYLNRLSSLLFVLALVVNKRTSNPNVIWR;
HAA.6

SEQ ID NO: 207

SKDSPIIEANGTLDELTSFIGEAKHYVDAEMQGILEEIQNDIYKIMGEIG
SKGKIEGISEERIKWLEGLISRYEEMVNLKSFVLPGGTLESAKLDVCRTI
ARRAERKVATVLREFGIGKEALVYLNRLSDLLFLLARVIEIE;
HAA. 7

SEQ ID NO: 208

GSMKKVEAIIRPEKLEIVKKALSDAGYVGMTVSEVKGRGVQGGIVERYRG
REYIVDLIPKVKIELVVKQADVDNVIDIICENARTGNPGDGKIFVIPVQR
VVRVRTKEEGAAALLEH;
HAB.1

SEQ ID NO: 209

ILVYSTFPNEEKALEIGRKLLEKRLIACFNAFEIRSGYWWKGEIVQDKEW
AAIFKTTEEKEKELYEELRKLHPYETPAIFTLKVENVLTEYMNWLRESV;
HAB.2

SEQ ID NO: 210

GSMKKVEAIIRPEKLEIVKKALSDAGYVGMTVSEVKGRGVQGGIVERYRG
REYIVDLIPKVKIELVVKEEDVDNVIDIICENARTGNPGDGKIFVIPVER
VVRVRTKEEGKEAL;
HAB.3

SEQ ID NO: 211

KKIEAIIRPFKLDEVKIALVNAGIVGMTVSEVRGFGRQKRGSEYTVEFLQ
KLKLEIVVEDAQVDTVIDKIVAAARTGENGDGKIFVSPVDQTIRIRTGEK
NADAI;
HAB.4

SEQ ID NO: 212

HLTPREFDKLVIHMLSDVALKRKNKGLKLNHPEAVAVLSAYVLDGAREGK
TVEEVMDGARSVLKADDVMDGVPDLLPLIQVEAVFSDGSRLVSLHNPI;
and
HAA.1c

SEQ ID NO: 227

MEEVVLITVPSEEVARTIAKALVEERLAACVNIVPGLTSIYRWQGEVVED
QELLLLVKTTTHAFPKLKERVKALHPYTVPEIVALPIAQGNQEYLDWLRE
NA.

As shown in the examples that follow, the polypeptides of this aspect of the invention can, for example, self-assemble into multimers ((dimers, trimers, pentamers, hexamers, etc.) In one embodiment, the polypeptides can self-assemble/organize into trimeric units to appropriately display receptor binding site (RBS) inhibitors in alignment with the RBS of the trimeric hemagglutinin of influenza. In one embodiment, polypeptide RBS inhibitors can be expressed as a fusion protein with the polypeptides of the invention. Conjugation (such as genetic fusion) of RBS inhibitors with the self-assembling polypeptides of the invention allows binding/interaction of multiple units of the RBS inhibitor with the hemagglutinin, resulting in a significant boost of affinity, and providing enhanced performance in diagnostic and therapeutic treatments. Studies indicate that the trimeric fusions inhibit infectivity of the influenza virus and can pull down and immobilize live virus for diagnostic purposes.

In various embodiments, the polypeptides comprise or consist of an amino acid sequence at least 91%, 92%, 93%, 94%, 95%, 96%, 97%, 98%, 99%, or 100% identical over their full length to at least one of the recited polypeptides of the invention.

In another embodiment, the isolated polypeptides further comprise an amino acid linker sequence at its C-terminus. The linker may be used, for example, as a means to conjugate the RBS inhibitor to the polypeptide of the invention. It should be noted that the RBS inhibitors can be conjugated to the polypeptides in any suitable manner, and thus the linkers are not required. In one embodiment, the linkers may be between 1 amino acid and 20 amino acids in length; in various other embodiments, the linker may be between 1-19, 1-18, 1-17, 1-16, 1-15, 1-14, 1-13, 1-12, 1-11, 1-10, 1-9, 1-8, 1-7, 1-6, 1-5, 1-4, 1-3, 1-2, 2-19, 2-18, 2-17, 2-16, 2-15, 2-14, 2-13, 2-12, 2-11, 2-10, 2-9, 7-8, 2-7, 2-6, 2-5, 2-4, or 2-3 amino acids in length.

The linkers between the oligomerization domain and the binding domain may be of any amino acid composition as deemed most suitable for a given use. In one non-limiting embodiment, the linker can be a poly-glycine linker. In various other non-limiting embodiments, a GS, GG, SNS, Ng, or GGGS (SEQ ID NO: 353) linker can be used. In various further embodiments, the linker may comprise or consist of Linker1 (CYIGN (SEQ ID NO: 347)); Linker2 (AGSQQ (SEQ ID NO: 348)); Linker3 (AGEEN (SEQ ID NO: 349)); Linker4 (AYSDQ K (SEQ ID NO: 350)); Linker5 (TGGGS (SEQ ID NO: 351), or Linker triLong (GGGGSGGS (SEQ ID NO: 352)). In one particular embodiment, the linker may be the triLong linker GGGGSGGS (SEQ ID NO: 352).

In various further non-limiting embodiments, the polypeptides of the invention including linkers may be:

HAA.1

SEQ ID 213

MEEVVLITVPSEEVARTIAKALVEERLAACVNIVPGLTSIYRWQGEVVED
QELLLLVKTTTHAFPKLKERVKALHPYTVPEIVALPIAQGNQEYLDWLRE
NTGS
HAA.1b

SEQ ID NO: 226

MEEVVLITVPSEEVARTIAKALVEERLAACVNIVPGLTSIYRWQGEVVED
QELLLLVKTTTHAFPKLKERVKALHPYTVPEIVALPIAQGNQEYLDWLRE
NAGS
HAA.1.1

SEQ ID NO: 214

MEEVVLITVPSEEVARTIAKALVEERLAACVNIVPGLTSIYRWQGEVVED
QELLLLVKTTTAAFPKLKERVKALHPYTVPEIVALPIAEGNREYLDWLQE
HAA.2

SEQ ID NO: 215

ALYFSGHMILVYSTFPNEEKALEIGRKLLEKRLIACFNAFEIRSGYWWKG
EIVQDKEWAAIFKTTQSKQKELYEELRKLHPYETPAIFTLKVENILTEYM
NWLAESVLGSGS
HAA.3

SEQ ID NO: 216

MKLIVAIVRPEKLNEVLKALFQAEVRGLTLSRVQGHGMELHEKVRLEIGV
SEPFVKPTVEAILKAARTGEVGDGKIFVLPVEKVYRIRTGEEAGS
HAA.3.1

SEQ ID NO: 217

MKLIVAIVRPEKLNEVLKALFQAEVRGLTLSRVQGHGGETERVETYRGTT
VKMELHEKVRLEIGVSEPFVKPTVEAILKAARTGEVGDGKIFVLPVEKVY
RIRTGEEAGS
HAA.4

SEQ ID NO: 218

KMEELFKKHKIVAVLRANSVEEAKEKALAVFEGGVHLIEITFTVPDADTV
IKELSFLKEKGAIIGAGTVTSVEQCRKAVESGAEFIVSPHLDEEISQFCK
EKGVFYMPGVMTPTELVKAMKLGHTILKLFPGQVVGPQFVKAMKGPFPNV
KFVPTGGVNLSNVAAWFAAGVLAVGVGSALVKGTPNNVREKAKAFVEAIR
GAGS
HAA.5

SEQ ID NO: 219

KDSEIVKALGDLDELNSVLGVVSSLYPSLSQVIQKLQNDIFSISSEIAGF
DMNFSDEKVKGIEELITNYSKELEPLSNFVLPGGHIASSFLHLARAVCRR
AERSVVTLLKESKAKEVHAKYLNRLSSLLFVLALVVNKRTSNPNVIWRGS
HAA.6

SEQ ID NO: 220

SKDSPIIEANGTLDELTSFIGEAKHYVDAEMQGILEEIQNDIYKIMGEIG
SKGKIEGISEERIKWLEGLISRYEEMVNLKSFVLPGGTLESAKLDVCRTI
ARRAERKVATVLREFGIGKEALVYLNRLSDLLFLLARVIEIEGS
HAA.7

SEQ ID NO: 221

GSMKKVEAIIRPEKLEIVKKALSDAGYVGMTVSEVKGRGVQGGIVERYRG
REYIVDLIPKVKIELVVKQADVDNVIDIICENARTGNPGDGKIFVIPVQR
VVRVRTKEEGAAALLEHGS
HAB.1

SEQ ID NO: 222

ILVYSTFPNEEKALEIGRKLLEKRLIACFNAFEIRSGYWWKGEIVQDKEW
AAIFKTTEEKEKELYEELRKLHPYETPAIFTLKVENVLTEYMNWLRESVG
G
HAB.2

SEQ ID NO: 223

GSMKKVEAIIRPEKLEIVKKALSDAGYVGMTVSEVKGRGVQGGIVERYRG
REYIVDLIPKVKIELVVKEEDVDNVIDIICENARTGNPGDGKIFVIPVER
VVRVRTKEEGKEALSNS
HAB.3

SEQ ID NO: 224

KKIEAIIRPFKLDEVKIALVNAGIVGMTVSEVRGFGRQKRGSEYTVEFLQ
KLKLEIVVEDAQVDTVIDKIVAAARTGENGDGKIFVSPVDQTIRIRTGEK
NADAIGGGS
HAB.4

SEQ ID NO: 225

HLTPREFDKLVIHMLSDVALKRKNKGLKLNHPEAVAVLSAYVLDGAREGK
TVEEVMDGARSVLKADDVMDGVPDLLPLIQVEAVFSDGSRLVSLHNPING

As shown in the examples that follow, the polypeptides of this aspect of the invention can, for example, self-assemble into multimers (dimers, trimers, pentamers, hexamers. etc.) Thus, in another embodiment the isolated polypeptide comprises a multimer of polypeptide units (dimer, Winter, pentamer, hexamer, etc.). In one embodiment, the multimer comprises a multimer of identical polypeptides of the invention; in another embodiment, the multimer may comprise different polypeptides of the invention.

As shown in the examples that follow, the polypeptides of this aspect of the invention can, for example, can self-assemble/organize into trimeric units to appropriately display receptor binding site (RHS) inhibitors in alignment with the RBS of the trimeric hemagglutinin of influenza. In one embodiment, polypeptide RBS inhibitors can be expressed as a fusion protein with the polypeptides of the invention. Thus, in another embodiment, the isolated polypeptides of the invention further comprise a hemagglutinin (HA) binding protein (referred to generically as a polypeptide RBS inhibitor herein) C-terminal to the linker.

The polypeptide RBS inhibitors described herein bind to the sialic acid binding or receptor site of influenza hemagglutinin (HA) protein and can thus be used, for example, to treat or detect/diagnose influenza infection. The polypeptides provide a cheaper, more selective alternative to currently used hemagglutinin binding antibodies, which are costly to produce. The polypeptides can also be used for in vivo biosensing applications, whereas the antibodies cannot because of their structurally necessary disulfide bonds and difficulty to express robustly.

In one embodiment, the polypeptide RBS inhibitor is a peptide at least 70% identical over the full length of the amino acid sequence in Table 1. in one embodiment, the polypeptide does not comprise the amino acid sequence of GIVNVPNPNNTKFQELAREAIQDYNKKQNAHLEFVENLNVKEQVVAGIMYYI TLAATDDAGKKKIYKAKIWVKEWEDKKVVEFKLV (SEQ ID NO: 129).

TABLE 1
SB52 genus (SEQ ID NO: 1)

| Residue | AAs |
|---|---|
| 1 | G, D, S, K, Q |
| 2 | I |
| 3 | V |
| 4 | N |
| 5 | V |
| 6 | P |
| 7 | N, K |
| 8 | P, C |
| 9 | N, K |
| 10 | N, T |
| 11 | T, K, P |
| 12 | K |
| 13 | F, Y |
| 14 | Q, R, I |
| 15 | Q, K, R |
| 16 | L |
| 17 | A, G |
| 18 | R, N, Y, T, S |
| 19 | N, R, S, Y, T, Q |
| 20 | A |
| 21 | I, V |
| 22 | A, S, Y, H |
| 23 | N, I, V |
| 24 | Y, V, W, M, I |
| 25 | N |
| 26 | D, Y, N, H |
| 27 | N, H, Y, Q, D |
| 28 | Q, K, E, D, W, Y |
| 29 | N, P, E, D |
| 30 | A, E |
| 31 | H |
| 32 | L |
| 33 | E, T, R, K, Q, S, Y, A, |
| | L, H, F, M, V, I, N |
| 34 | F |
| 35 | V |
| 36 | E, H, Q |
| 37 | N |
| 38 | L, M |
| 39 | N |
| 40 | V, C |
| 41 | K |
| 42 | E, D, K |
| 43 | Q, K |
| 44 | Any amino acid, or |
| | absent |
| 45 | Any amino acid, or |
| | absent |
| 46 | G (Position 46 and 47 |
| | are set so that one of |
| | them is glycine and |
| | the other is any of the |
| | following amino |
| | acids; |
| | N, H, D, S, K, Y, H, A, V, T, |
| | I, Q, D, E, M, L, F), or |
| | absent |
| 47 | G (Position 46 and 47 |
| | are set so that one of |
| | them is glycine and |
| | the other is any of the |
| | following amino |
| | acids; |
| | N, H, D, S, K, Y, H, A, V, T, |
| | I, Q, D, E, M, L, F, K) |
| 48 | Any amino acid |
| 49 | M, L, Y, W |
| 50 | Y |
| 51 | Y, W |
| 52 | I |
| 53 | T |
| 54 | L |
| 55 | A |
| 56 | A |
| 57 | T |
| 58 | D |
| 59 | D, Q |
| 60 | A |
| 61 | G |
| 62 | K, E |
| 63 | K |
| 64 | K, S, A, W, L, M, Q, |
| | G |
| 65 | I |
| 66 | Y |
| 67 | K, S, A, Y, W, M, F |
| 68 | A, K, Y, T |
| 69 | K |
| 70 | I, V, L |
| 71 | A, G |
| 72 | V |
| 73 | V or is absent |
| 74 | D, E |
| 75 | S |
| 76 | A, S |
| 77 | G |
| 78 | W |
| 79 | E, V, I, K, A, T, L |
| 80 | G, V, A |
| 81 | V, L |
| 82 | Any amino acid |
| 83 | E |
| 84 | F |
| 85 | K, A, E, D |
| 86 | L |
| 87 | V, I, M |

In one embodiment, the polypeptide RBS inhibitors are at least 70% identical with the amino acid sequence of SEQ ID NO:1 (Table 1), over its full length. As disclosed in detail in the examples that follow, numerous HA-binding polypeptides (i.e.: polypeptide RBS inhibitors) of this embodiment have been identified, and the inventors have discovered that residues 18-27, 44-48, and 70-82 primarily make up the interface for HA protein binding; these regions have been subjected to extensive further analysis to identify variability within these regions. The remaining residues (1-17, 28-43, 49-69, and 83-87) can be modified, as these residues are not involved in the HA protein interface. Such modifications may comprise, for example, conservative amino acid substitutions. Thus, in one preferred embodiment, the polypeptide RBS inhibitors are at least 70% identical with the amino acid sequence of SEQ ID NO: 1, wherein variability is within residues 18-27, 44-48, and/or 70-82. In various embodiments, the polypeptide RBS inhibitors are at least 75%, 80%, 85%, 90%, 91%, 92%, 93%, 94%, 95%, 96%, 97%, 98%, 99%, or completely identical with to the amino acid sequence of SEQ ID NO:1 (Table 1), over its full length. In various preferred embodiments, the polypeptide RBS inhibitors are at least 75%, 80%, 85%, 90%, 91%, 92%, 93%, 94%, 95%, 96%, 97%, 98%, 99%, or completely identical with the amino acid sequence of SEQ ID NO:1, wherein variability is wherein residues 18-27, 44-48, and/or 70-82.

In one embodiment, the polypeptide RBS inhibitors comprise or consist of a polypeptide at least 70% identical the amino acid sequence in Table 2.

TABLE 2
(SEQ ID NO: 2)

| Residue | AAs |
|---|---|
| 1 | G, D |
| 2 | I |
| 3 | V |
| 4 | N |
| 5 | V |
| 6 | P |
| 7 | N |
| 8 | P, C |
| 9 | N |
| 10 | N, T |
| 11 | T |
| 12 | K |
| 13 | F, Y |
| 14 | Q |
| 15 | Q |
| 16 | L |
| 17 | A |
| 18 | R |
| 19 | N, R, S, I, Y |
| 20 | A |
| 21 | I |
| 22 | A |
| 23 | N, I, V |
| 24 | Y |
| 25 | N |
| 26 | D, Y, N, H |
| 27 | N, H, Y, Q |
| 28 | Q |
| 29 | N |
| 30 | A |
| 31 | H |
| 32 | L |
| 33 | E |
| 34 | F |
| 35 | V |
| 36 | E |
| 37 | N |
| 38 | L |
| 39 | N |
| 40 | V, C |
| 41 | K |
| 42 | E, D |
| 43 | Q |
| 44 | V, L, I, T, A, S, W, |
| 45 | T, S, D, G, A, N, Y |
| 46 | G, Y, F, A, L, S, E |
| | (wherein one of residue |
| | 46 or 47 is G) |
| 47 | M, K, G, L, H, Q, E, D, |
| | V, S (wherein one of |
| | residue 46 or 47 is G) |
| 48 | N, T, V, A, I, D, G, V, |
| | S |
| 49 | M |
| 50 | Y |
| 51 | Y |
| 52 | I |
| 53 | T |
| 54 | L |
| 55 | A |
| 56 | A |
| 57 | T |
| 58 | D |
| 59 | D |
| 60 | A |
| 61 | G |
| 62 | K |
| 63 | K |
| 64 | K |
| 65 | I |
| 66 | Y |
| 67 | K |
| 68 | A |
| 69 | K |
| 70 | I, V |
| 71 | A, G |
| 72 | V |
| 73 | V or is absent |
| 74 | D, E |
| 75 | S |
| 76 | A |
| 77 | G |
| 78 | W |
| 79 | E, V, I, K, A, T |
| 80 | G, V, A, E |
| 81 | V |
| 82 | T, E, K, R, D, A, S, N |
| 83 | E |
| 84 | F |
| 85 | K |
| 86 | L |
| 87 | V |

The polypeptide RBS inhibitors are at least 70% identical with the amino acid sequence of SEQ ID NO:2 (Table 2), over its full length. In one preferred embodiment, the polypeptide RBS inhibitors are at least 70% identical with the amino acid sequence of SEQ ID NO:2, wherein variability is within residues 18-27, 44-48, and/or 70-82. In various embodiments. the polypeptide RBS inhibitors are at least 75%, 80%, 85%, 90%, 91%, 92%, 93%, 94%, 95%, 96%, 97%, 98%, 99%, or completely identical with to the amino acid sequence of SEQ ID NO:2, over its full length. In various preferred embodiments, the polypeptide RBS inhibitors are at least 75%, 80%, 85%, 90%, 91%, 92%, 93%, 94%, 95%, 96%, 97%, 98%, 99%, or completely identical with the amino acid sequence of SEQ ID NO:2, wherein variability is within residues 18-27, 44-48, and/or 70-82.

In another embodiment, the polypeptide RBS inhibitors comprise or consist of a polypeptide at least 70% identical to the amino acid sequence in Table 3.

TABLE 3
(SEQ ID NO: 3)

| Residue | AAs |
|---|---|
| 1 | G |
| 2 | I |
| 3 | V |
| 4 | N |
| 5 | V |
| 6 | P |
| 7 | N |
| 8 | P, C |
| 9 | N |
| 10 | T |
| 11 | T |
| 12 | K |
| 13 | Y |
| 14 | Q |
| 15 | Q |
| 16 | L |
| 17 | A |
| 18 | R |
| 19 | N, R, S, I, Y |
| 20 | A |
| 21 | I |
| 22 | A |
| 23 | I, V, N |
| 24 | Y |
| 25 | N |
| 26 | Y, N, D, H |
| 27 | N, H, Y, Q |
| 28 | Q |
| 29 | N |
| 30 | A |
| 31 | H |
| 32 | L |
| 33 | E |
| 34 | F |
| 35 | V |
| 36 | E |
| 37 | N |
| 38 | L, M |
| 39 | N |
| 40 | V, C |
| 41 | K |
| 42 | E, D |
| 43 | Q |
| 44 | V, L, I, T, A, S, W |
| 45 | T, S, D, G, A |
| 46 | G, Y, L, H, V, S, E |
| | (wherein one of |
| | residue 46 or 47 is G) |
| 47 | M, K, G, L, H, Q, E, |
| | Y, D, S (wherein one |
| | of residue 46 or 47 is |
| | G) |
| 48 | N, T, V, A, I, D, G, V, S |
| 49 | M |
| 50 | Y |
| 51 | Y |
| 52 | I |
| 53 | T |
| 54 | L |
| 55 | A |
| 56 | A |
| 57 | T |
| 58 | D |
| 59 | D |
| 60 | A |
| 61 | G |
| 62 | K |
| 63 | K |
| 64 | K |
| 65 | I |
| 66 | Y |
| 67 | K |
| 68 | A |
| 69 | K |
| 70 | I, V |
| 71 | A, G |
| 72 | V |
| 73 | V or is absent |
| 74 | E |
| 75 | S |
| 76 | A |
| 77 | G, |
| 78 | W |
| 79 | E, V, I, K, A, T |
| 80 | G, A, V, E |
| 81 | V |
| 82 | T, E, S, R, K, N, A |
| 83 | E |
| 84 | F |
| 85 | K |
| 86 | L |
| 87 | V |

The polypeptide RBS inhibitors are at least 70% identical with the amino acid sequence of SEQ ID NO:3 (Table 3), over its full length. In one preferred embodiment, the polypeptide RBS inhibitors are at least 70% identical with the amino acid sequence of SEQ ID NO:3, wherein variability is within residues 18-27, 44-48, and/or 70-82. In various embodiments, the polypeptide RBS inhibitors are at least 75%, 80%, 85%, 90%, 91%, 92%, 93%, 94%, 95%, 96%, 97%, 98%, 99%, or completely identical with to the amino acid sequence of SEQ ID NO:3, over its full length. In various preferred embodiments, the polypeptide RBS inhibitors are at least 75%, 80%, 85%, 90%, 91%, 92%, 93%, 94% 95%, 96%, 97%, 98%, 99%, or completely identical with the amino acid sequence of SEQ ID NO:3, wherein variability is within residues 18-27, 44-48, and/or 70-82.

In various further embodiments, the polypeptide RBS inhibitors comprise or consist of a peptide with an amino acid sequence selected from the group consisting of:

SB52

(SEQ ID NO: 11)

GIVNVPN(P/C)NNTKFQQLARNAIANYNDNQNAHLEFVENLN(V/C)KE
QVTGGIMYYITLTDDAGKKKIYKAKIAVVDSAGWEGVTEFKLV;
Variant SB52-3 (G47D, N27Y)

(SEQ ID NO: 12)

GIVNVPN(P/C)NNTKFQQLARNAIANYNDYQNAHLEFVENLN(V/C)KE
QVTGDIMYYITLAATDDAGKKKIYKAKIAVVDSAGWEGVTEFKLV;
Variant H2 SB52-4 (N19Y, E79V)

(SEQ ID NO: 13)

GIVNVPN(P/C)NNTKFQQLARYAIANYNDNQNAHLEFVENLN(V/C)KE
QVTGGIMYYITLAATDDAGKKKIYKAKIAVVDSAGWVGNFTEFKLV;
Variant H2 SB52-13 (N7K, N19Y, E79V)

(SEQ ID NO: 14)

GIVNVPK(P/C)NNTKFQQLARYAIANYNDNQNAHLEFVENLN(V/C)KE
QVTGGIMYYITLAATDDAGKKKIYKAKIAVVDSAGWVGVTEFKLV;
52solo1

(SEQ ID NO: 15)

GIVNVPN(P/C)NTTKYQQLARSAIAIYNYHQNAHLEFVENLN(V/C)KE
QVGGMNMYYITLAATDDAGKKKIYKAKVGVVESAGWKGVEEFKLV;
52solo2

(SEQ ID NO: 16)

GIVNVPN(P/C)NTTKYQQLARSAIAVYNYHQNAHLEFVENLN(V/C)KE
QIAGKTMYYITLAATDDAGKKKIYKAKVGVVESAGWEGVEEFKLV;
52solo3

(SEQ ID NO: 17)

GIVNVPN(P/C)NTTKYQQLARIAIAVYNNYQNAHLEFVENLN(V/C)KE
QSGGKVMYYITLAATDDAGKKKIYKAKVGVVESAGWIGVEEFKLV;
52solo8

(SEQ ID NO: 18)

GIVNVPN(P/C)NTTKYQQLARSAIAVYNNHQNAHLEFVENLN(V/C)KE
QAGGKAMYYITLAATDDAGKKKIYKAKIGVVESAGWEGVEEFKLV;
52sing2

(SEQ ID NO: 19)

GIVNVPN(P/C)NTTKYQQLARRAIAIYNNNQNAHLEFVENLN(V/C)KE
QTGLGIMYYITLAATDDAGKKKIYKAKIAVVESAGWVAVSEFKLV;
52solo8a

(SEQ ID NO: 20)

GIVNVPN(P/C)NTTKYQQLARSAIAVYNNHQNAHLEFVENLN(V/C)KE
QAGGKAMYYITLAATDDAGKKKIYKAKIGVVESAGWEGVEEFKLV;
52solo8b

(SEQ ID NO: 21)

GIVNVPN(P/C)NTTKYQQLARSAIAVYNYHQNAHLEFVENLN(V/C)KE
QIAGKTMYYITLAATDDAGKKKIYKAKVGVVESAGWEGVEEFKLV;
52soloc

(SEQ ID NO: 22)

GIVNVPN(P/C)NTTKYQQLARSAIAVYNNHQNAHLEFVENLN(V/C)KE
QAGGKAMYYITLAATDDAGKKKIYKAKIGVVESAGWEGVEEFKLV;
52sing2

(SEQ ID NO: 23)

GIVNVPN(P/C)NTTKYQQLARRAIAIYNNNQNAHLEFVENLN(V/C)KE
QTGLGIMYYITLAATDDAGKKKIYKAKIAVVESAGWVAVSEFKLV;
52sing2W

(SEQ ID NO: 24)

GIVNVPN(P/C)NTTKYQQLARRAIAIYNNNQNAHLEFVENLN(V/C)KE
QWGLGIMYYITLAATDDAGKKKIYKAKIAVVESAGWVAVSEFKLV;
52sing2D

(SEQ ID NO: 25)

GIVNVPN(P/C)NTTKYQQLARRAIAIYNNNQNAHLEFVENLN(V/C)KD
QTGLGIMYYITLAATDDAGKKKIYKAKIAVVESAGWVAVSEFKLV;
52alt1-3

(SEQ ID NO: 26)

GIVNVPN(P/C)NTTKYQQLARSAIAIYNNHQNAHLEFVENLN(V/C)KE
QVSYGAMYYITLAATDDAGKKKIYKAKVGVVESAGWVGVEEFKLV;
52alt1-4

(SEQ ID NO: 27)

GIVNVPN(P/C)NTTKYQQLARSAIAVYNYHQNAHLEFVENLN(V/C)KE
QIDYGAMYYITLAATDDAGKKKIYKAKIGVVESAGWIGVEEFKLV;
52vic5-2

(SEQ ID NO: 28)

GIVNVPN(P/C)NTTKYQQLARRAIAIYNNNQNAHLEFVENLN(V/C)KE
QWGLGIMYYITLAATDDAGKKKIYKAKIAVVESAGWVAVSEFKLV;
52vic5-3

(SEQ ID NO: 29)

GIVNVPN(P/C)NTTKYQQLARSAIAIYNNNQNAHLEFVENLN(V/C)KE
QLNGYDMYYITLAATDDAGKKKIYKAKIGVVESAGWTGVNEFKLV;
52vic5-4

(SEQ ID NO: 30)

GIVNVPN(P/C)NTTKYQQLARSAIAIYNHYQNAHLEFVENLN(V/C)KE
QITGYDMYYITLAATDDAGKKKIYKAKVAVVESAGWEVVAEFKLV;
52del1

(SEQ ID NO: 31)

GIVNVPN(P/C)NTTKYQQLARRAIAVYNYYQNAHLEFVENLN(V/C)KE
QSSGLDMYYITLAATDDAGKKKIYKAKIAVESAGWIVVTEFKLV;
52del4

(SEQ ID NO: 32)

GIVNVPN(P/C)NTYKYQQLARRAIAVYNYYQNAHLEFVENLN(V/C)KE
QLTGHGMYYITLAATDDAGKKKIYKAKVAVESAGWIVVTEFKLV;
52del2

(SEQ ID NO: 33)

GIVNVPN(P/C)NTTKYQQLARRAIAVYNYYQNAHLEFVENLN(V/C)KE
QAGGQVMYYITLAATDDAGKKKIYKAKVAVESAGWIVVTEFKLV;
52del3

(SEQ ID NO: 34)

GIVNVPN(P/C)NTTKYQQLARRAIAVYNYYQNAHLEFVENLN(V/C)KE
QAAGEIMYYITLAATDDAGKKKIYKAKIAVESAGWIVVREFKLV;
52del5

(SEQ ID NO: 35)

GIVNVPN(P/C)NTTKYQQLARRAIAVYNYYQNAHLEFVENLN(V/C)KE
QAAGEIMYYITLAATDDAGKKKIYKAKIAVESAGWIVVREFKLV;
52del6

(SEQ ID NO: 36)

GIVNVPN(P/C)NTTKYQQLARIAIAIYNYHQNAHLEFVENLN(V/C)KE
QSGHGTMYYITLAATDDAGKKKIYKAKVGVVESAGWIGVTEFKLV;
52del7

(SEQ ID NO: 37)

GIVNVPN(P/C)NTTKYQQLARIAIAVYNHHQNAHLEFVENLN(V/C)KE
QISVGTMYYITLAATDDAGKKKIYKAKVGVVESAGWIEVEEFKLV;
52del8

(SEQ ID NO: 38)

GIVNVPN(P/C)NTTKYQQLARRAIAVYNYYQNAHLEFVENLN(V/C)KE
QLTLGVMYYITLAATDDAGKKKIYKAKVAV-ESAGWIVVTEFKLV;
52del9

(SEQ ID NO: 39)

GIVNVPN(P/C)NTTKYQQLARRAIAVYNYYQNAHLEFVENLN(V/C)KE
QSSSGNMYYITLAATDDAGKKKIYKAKIAV-ESAGWIVVKEFKLV;
52-N19S

(SEQ ID NO: 131)

GIVNVEN(P/C)NNTKFQQLARSAIANYNDYQNAHLEFVENLN(V/C)KE
QVTGDIMYYITLAATDDAGKKKIYKAKIAVVDSAGWEGVTEFKLV
52-N19Y

(SEQ ID NO: 132)

GIVNVPN(P/C)NNTKFQQLARYAIANYNDYQNAHLEFVENLN(V/C)KE
QVTGDIMYYITLAATDDAGKKKIYKAKIAVVDSAGWEGVTEFKLV
52-L38M

(SEQ ID NO: 133)

GIVNVPN(P/C)NNTKFQQLARNAIANYNDYQNAHLEFVENMN(V/C)KE
QVTGDIMYYITLAATDDAGKKKIYKAKIAVVDSAGWEGVTEFKLV
52-G47D

(SEQ ID NO: 134)

GIVNVPN(P/C)NNTKIFQQLARNAIANYNDYQNAHLEFVENLN(V/C)K
EQVTDDIMYYITLAATDDAGKKKIYKAKIAVVDSAGWEGVTEFKLV
52NC-1

(SEQ ID NO: 95)

GIVNVPN(P/C)NTTKYQQLARIAIAVYNHHQNAHLEFVENLN(V/C)KE
QLGEGDMYYITLAATDDAGKKKIYKAKVGVVESAGWTGVEEFKLV;
52NC-2

(SEQ ID NO: 96)

GIVNVPN(P/C)NTTKYQQLARSAIAIYNYHQNAHLEFVENLN(V/C)KE
QLGEGDMYYITLAATDDAGKKKIYKAKIGVVESAGWTGVEEFKLV;
52NC-8

(SEQ ID NO: 97)

GIVNVPN(P/C)NTTKYQQLARSAIAIYNYHQNAHLEFVENLN(V/C)KE
QIGEGSMYYITLAATDDAGKKKIYKAKVGVVESAGWEGVEEFKLV;
52solo6

(SEQ ID NO: 126)

GIVNVPN(P/C)NTTKYQQLARRAIAIYNHNQNAHLEFVENLN(V/C)KE
QIDYGSMYYITLAATDDAGKKKIYKAKVGVVESSGWTGVEEFKLV;
Hsc1

(SEQ ID NO: 135)

GIVNVPNCNTTKYQQLARTAVAIYNYHEQAHLTFVENLNCKEQLGEGDYY
YITLAATDDAGKKAIYEAKIGVVESAGWTGVEEFKLV;
Hsc3

(SEQ ID NO: 136)

GIVNVPNCNTTKYQQLARTAIAIMNYHQQAHLTFVENLNCKEQLGEGDYY
YITLAATDDAGKKAIYEAKIGVVESAGWTGVEEFKLV;
Hsc12

(SEQ ID NO: 137)

GIVNVPNCNTTKYQQLARTAIAIVNCHEHAHLTFVENLNCKEQLGEGDIY
YITLAATDDAGKKSIYEAKIGVVESAGWTGVEEFKLV;
HscA

(SEQ ID NO: 138)

GIVNVPNCNTTKYQQLARTAVAIYNYHKPAHLTFVENLNCKEQLGEGDIY
YITLAATDDAGKKSIYSAKIGVVESAGWTGVEEFKLV;
HscB

(SEQ ID NO: 139)

GIVNVPNCNTTKYQQLARTAVAIYNYHNHAHLTFVENLNCKEQLGEGDLY
YITLAATDDAGKKAIYTAKIGVVESAGWTGVEEFKLV;
Hsc1-3357

(SEQ ID NO: 140)

GIVNVPNPNTTKYQQLARTAVAIYNYHEQAHLCFVENLNVKEQLGEGDYY
YITLAACDDAGKKAIYEAKIGVVESAGWTGVEEFKLV;
Hsc1-EA

(SEQ ID NO: 141)

GIVNVPNCNTTKYQQLARTAVAIYNYHEQAHLTFVENLNCKEQGNYYYIT
LAATDDAGKKAIYEAKIGVVESAGWTGVEEFKLV;
Hscl-3357EA

(SEQ ID NO: 142)

GIVNVPNPNTTKYQQLARTAVAIYNYHEQAHLCFVENLNVKEQGNYYYIT
LAACDDAGKKAIYEAKIGVVESAGWTGVEEFKLV;
W-64

(SEQ ID NO: 143)

GIVNVPNCNTTKYQQLARTAVAIYNYHEQAHLTFVENLNCKEQLGEGDYY
YITLAATDDAGKKWIYEAKIGVVESAGWTGVEEFKLV;
L-64

(SEQ ID NO: 228)

GIVNVPNCNTTKYQQLARTAVAIYNYHEQAHLTFVENLNCKEQLGEGDYY
YITLAATDDAGKKLIYEAKIGVVESAGWTGVEEFKLV;
P-29

(SEQ ID NO: 144)

GIVNVPNCNTTKYQQLARTAVAIYNYHEPAHLTFVENLNCKEQLGEGDYY
YITLAATDDAGKKAIYEAKIGVVESAGWTGVEEFKLV;
R-14

(SEQ ID NO: 145)

GIVNVPNCNTTKYRQLARTAVAIYNYHEQAHLTFVENLNCKEQLGEGDYY
YITLAATDDAGKKAIYEAKIGVVESAGWTGVEEFKLV;
K-43

(SEQ ID NO: 146)

GIVNVPNCNTTKYQQLARTAVAIYNYHEQAHLTFVENLNCKEKLGEGDYY
YITLAATDDAGKKAIYEAKIGVVESAGWTGVEEFKLV;
T-68

(SEQ ID NO: 147)

GIVNVPNCNTTKYQQLARTAVAIYNYHEQAHLTFVENLNCKEQLGEGDYY
YITLAATDDAGKKAIYETKIGVVESAGWTGVEEFKLV;
K-11

(SEQ ID NO: 148)

GIVNVPNCNTKKYQQLARTAVAIYNYHEQAHLTFVENLNCKEQLGEGDYY
YITLAATDDAGKKAIYEAKIGVVESAGWTGVEEFKLV;
Q-59

(SEQ ID NO: 149)

GIVNVPNCNTTKYQQLARTAVAIYNYHEQAHLTFVENLNCKEQLGEGDYY
YITLAATDQAGKKAIYEAKIGVVESAGWTGVEEFKLV;
L-49

(SEQ ID NO: 150)

GIVNVPNCNTTKYQQLARTAVAIYNYHEQAHLTFVENLNCKEQLGEGDLY
YITLAATDDAGKKAIYEAKIGVVESAGWTGVEEFKLV;
Y-33

(SEQ ID NO: 151)

GIVNVPNCNTTKYQQLARTAVAIYNYHEQAHLYFVENLNCKEQLGEGDYY
YITLAATDDAGKKAIYEAKIGVVESAGWTGVEEFKLV;
N-33

(SEQ ID NO: 152)

GIVNVPNCNTTKYQQLARTAVAIYNYHEQAHLNFVENLNCKEQLGEGDYY
YITLAATDDAGKKAIYEAKIGVVESAGWTGVEEFKLV;
W-51

(SEQ ID NO: 153)

GIVNVPNCNTTKYQQLARTAVAIYNYHEQAHLTFVENLNCKEQLGEGDYY
WITLAATDDAGKKAIYEAKIGVVESAGWTGVEEFKLV;
A-33

(SEQ ID NO: 154)

GIVNVPNCNTTKYQQLARTAVAIYNYHEQAHLAFVENLNCKEQLGEGDYY
YITLAATDDAGKKAIYEAKIGVVESAGWTGVEEFKLV;
I-87

(SEQ ID NO: 155)

GIVNVPNCNTTKYQQLARTAVAIYNYHEQAHLTFVENLNCKEQLGEGDYY
YITLAATDDAGKKAIYEAKIGVVESAGWTGVEEFKLI;
A-85

(SEQ ID NO: 156)

GIVNVPNCNTTKYQQLARTAVAIYNYHEQAHLTFVENLNCKEQLGEGDYY
YITLAATDDAGKKAIYEAKIGVVESAGWTGVEEFALV;
I-14

(SEQ ID NO: 157)

GIVNVPNCNTTKYIQLARTAVAIYNYHEQAHLTFVENLNCKEQLGEGDYY
YITLAATDDAGKKAIYEAKIGVVESAGWTGVEEFKLV;
K-47

(SEQ ID NO: 158)

GIVNPNCNTTKYQQLARTAVAIYNYHEQAHLTFVENLNCKEQLGEKDYYY
ITLAATDDAGKKAIYEAKIGVVESAGWTGVEEFKLV;
E-62

(SEQ ID NO: 159)

GIVNVPNCNTTKYQQLARTAVAIYNHEQAHLTFVENLNCKEQLGEGDYYY
ITLAATDDAGEKAIYEAKIGVVESAGWTGVEEFKLV;
M-87

(SEQ ID NO: 160)

GIVNVPNCNTTKYQQLARTAVAIYNYHEQAHLTFVENLNCKEQLGEGDYY
YITLAATDDAGKKAIYEAKIGVVESAGWTGVEEFKLV;
D-28

(SEQ ID NO: 161)

GIVNVPNCNTTKYQQLARTAVAIYNYHDQAHLTFVENLNCKEQLGEGDYY
YITLAATDDAGKKAIYEAKIGVVESAGWTGVEEFKLV;
S-67

(SEQ ID NO: 162)

GIVNVPNCNTTKYQQLARTAVAIYNYHEQAHLTFVENLNCKEQLGEGDYY
YITLAATDDAGKKAIYSAKIGVVESAGWTGVEEFKLV;
E-85

(SEQ ID NO: 163)

GIVNVPNCNTTKYQQLARTAVAIYNYHEQAHLTFVENLNCKEQLGEGDYY
YITLAATDDAGKKAIYEAKIGVVESAGWTGVEEFELV;
A-67

(SEQ ID NO: 164)

GIVNVPNCNTTKYQQLARTAVAIYNYHEQAHLTFVENLNCKEQLGEGDYY
YITLAATDDAGKKAIYAAKIGVVESAGWTGVEEFKLV;
H-36

(SEQ ID NO: 165)

GIVNVPNCNTTKYQQLARTAVAIYNYHEQAHLTFVHNLNCKEQLGEGDYY
YITLAATDDAGKKAIYEAKIGVVESAGWTGVEEFKLV;
L-81

(SEQ ID NO: 166)

GIVNVPNCNTTKYQQLARTAVAIYNYHEQAHLTFVENLNCKEQLGEGDYY
YITLAATDDAGKKAIYEAKIGVVESAGWTGLEEFKLV;
E-30

(SEQ ID NO: 167)

GIVNVPNCNTTKYQQLARTAVAIYNYHEQEHLTFVENLNCKEQLGEGDYY
YITLAATDDAGKKAIYEAKIGVVESAGWTGVEEFKLV;
P-11

(SEQ ID NO: 168)

GIVNVPNCNTPKYQQLARTAVAIYNYHEQAHLTFVENLNCKEQLGEGDYY
YITLAATDDAGKKAIYEAKIGVVESAGWTGVEEFKLV;
M-64

(SEQ ID NO: 169)

GIVNVPNCNTTKYQQLARTAVAIYNYHEQAHLTFVENLNCKEQLGEGDYY
YITLAATDDAGKKMIYEAKIGVVESAGWTGVEEFKLV;
S-64

(SEQ ID NO: 170)

GIVNVPNCNTTKYQQLARTAVAIYNYHEQAHLTFVENLNCKEQLGEGDYY
YITLAATDDAGKKSIYEAKIGVVESAGWTGVEEFKLV;
K-28

(SEQ ID NO: 171)

GIVNVPNCNTTKYQQLARTAVAIYNYHKQAHLTFVENLNCKEQLGEGDYY
YITLAATDDAGKKAIYEAKIGVVESAGWTGVEEFKLV;
S-22

(SEQ ID NO: 172)

GIVNVPNCNTTKYQQLARTAVSIYNYHEQAHLTFVENLNCKEQLGEGDYY
YITLAATDDAGKKAIYEAKIGVVESAGWTGVEEFKLV;
D-27

(SEQ ID NO: 173)

GIVNVPNCNTTKYQQLARTAVAIYNYDEQAHLTFVENLNCKEQLGEGDYY
YITLAATDDAGKKAIYEAKIGVVESAGWTGVEEFKLV;
L-33

(SEQ ID NO: 174)

GIVNVPNCNTTKYQQLARTAVAIYNYHEQAHLLFVENLNCKEQLGEGDYY
YITLAATDDAGKKAIYEAKIGVVESAGWTGVEEFKLV;
I-33

(SEQ ID NO: 175)

GIVNVPNCNTTKYQQLARTAVAIYNYHEQAHLIFVENLNCKEQLGEGDYY
YITLAATDDAGKKAIYEAKIGVVESAGWTGVEEFKLV;
W-28

(SEQ ID NO: 176)

GIVNVPNCNTTKYQQLARTAVAIYNYHWQAHLTFVENLNCKEQLGEGDYY
YITLAATDDAGKKAIYEAKIGVVESAGWTGVEEFKLV;
Q-64

(SEQ ID NO: 177)

GIVNVPNCNTTKYQQLARTAVAIYNYHEQAHLTFVENLNCKEQLGEGDYY
YITLAATDDAGKKQIYEAKIGVVESAGWTGVEEFKLV;
Y-67

(SEQ ID NO: 178)

GIVNVPNCNTTKYQQLARTAVAIYNYHEQAHLTFVENLNCKEQLGEGDYY
YITLAATDDAGKKAIYYAKIGVVESAGWTGVEEFKLV;
Y-22

(SEQ ID NO: 179)

GIVNVPNCNTTKYQQLARTAVYIYNYHEQAHLTFVENLNCKEQLGEGDYY
YITLAATDDAGKKAIYEAKIGVVESAGWTGVEEFKLV;
G-64

(SEQ ID NO: 180)

GIVNVPNCNTTKYQQLARTAVAIYNYHEQAHLTFVENLNCKEQLGEGDYY
YITLAATDDAGKKGIYEAKIGVVESAGWTGVEEFKLV;
W-67

(SEQ ID NO: 181)

GIVNVPNCNTTKYQQLARTAVAIYNYHEQAHLTFVENLNCKEQLGEGDYY
YITLAATDDAGKKAIYWAKIGVVESAGWTGVEEFKLV;
M-67

(SEQ ID NO: 182)

GIVNVPNCNTTKYQQLARTAVAIYNYHEQAHLTFVENLNCKEQLGEGDYY
YITLAATDDAGKKAIYMAKIGVVESAGWTGVEEFKLV;
D-29

(SEQ ID NO: 183)

GIVNVPNCNTTKYQQLARTAVAIYNYHEDAHLTFVENLNCKEQLGEGDYY
YITLAATDDAGKKAIYEAKIGVVESAGWTGVEEFKLV;
H-33

(SEQ ID NO: 184)

GIVNVPNCNTTKYQQLARTAVAIYNYHEQAHLHFVENLNCKEQLGEGDYY
YITLAATDDAGKKAIYEAKIGVVESAGWTGVEEFKLV;
D-85

(SEQ ID NO: 185)

GIVNVPNCNTTKYQQLARTAVAIYNYHEQAHLTFVENLNCKEQLGEGDYY
YITLAATDDAGKKAIYEAKIGVVESAGWTGVEEFDLV;
M-24

(SEQ ID NO: 186)

GIVNVPNCNTTKYQQLARTAVAIMNYHEQAHLTFVENLNCKEQLGEGDYY
YITLAATDDAGKKAIYEAKIGVVESAGWTGVEEFKLV;
A-64

(SEQ ID NO: 187)

GIVNVPNCNTTKYQQLARTAVAIYNYHEQAHLTFVENLNCKEQLGEGDYY
YITLAATDDAGKKAIYEAKIGVVESAGWTGVEEFKLV;
Y-49

(SEQ ID NO: 188)

GIVNVPNCNTTKYQQLARTAVAIYNYHEQAHLTFVENLNCKEQLGEGDYY
YITLAATDDAGKKAIYEAKIGVVESAGWTGVEEFKLV;
L-79

(SEQ ID NO: 189)

GIVNVPNCNTTKYQQLARTAVAIYNYHEQAHLTFVENLNCKEQLGEGDYY
YITLAATDDAGKKAIYEAKIGVVESAGWLGVEEFKLV;
S-33

(SEQ ID NO: 190)

GIVNVPNCNTTKYQQLARTAVAIYNYHEQAHLSFVENLNCKEQLGEGDYY
YITLAATDDAGKKAIYEAKIGVVESAGWTGVEEFKLV;
W-24

(SEQ ID NO: 191)

GIVNVPNCNTTKYQQLARTAVAIWNYHEQAHLTFVENLNCKEQLGEGDYY
YITLAATDDAGKKAIYEAKIGVVESAGWTGVEEFKLV;
I-24

(SEQ ID NO: 192)

GIVNVPNCNTTKYQQLARTAVAIINYHEQAHLTFVENLNCKEQLGEGDYY
YITLAATDDAGKKAIYEAKIGVVESAGWTGVEEFKLV;
Q-19

(SEQ ID NO: 193)

GIVNVPNCNTTKYQQLARQAVAIYNYHEQAHLTFVENLNCKEQLGEGDYY
YITLAATDDAGKKAIYEAKIGVVESAGWTGVEEFKLV;
V-24

(SEQ ID NO: 194)

GIVNVPNCNTTKYQQLARTAVAIVNYHEQAHLTFVENLNCKEQLGEGDYY
YITLAATDDAGKKAIYEAKIGVVESAGWTGVEEFKLV;
F-33

(SEQ ID NO: 195)

GIVNVPNCNTTKYQQLARTAVAIYNYHEQAHLFFVENLNCKEQLGEGDYY
YITLAATDDAGKKAIYEAKIGVVESAGWTGVEEFKLV;
E-29

(SEQ ID NO: 229)

GIVNVPNCNTTKYQQLARTAVAIYNYHEEAHLTFVENLNCKEQLGEGDYY
YITLAATDDAGKKAIYEAKIGVVESAGWTGVEEFKLV;
M-33

(SEQ ID NO: 196)

GIVNVPNCNTTKYQQLARTAVAIYNYHEQAHLMFVENLNCKEQLGEGDYY
YITLAATDDAGKKAIYEAKIGVVESAGWTGVEEFKLV;
K-15

(SEQ ID NO: 197)

GIVNVPNCNTTKYQKLARTAVAIYNYHEQAHLTFVENLNCKEQLGEGDYY
YITLAATDDAGKKAIYEAKIGVVESAGWTGVEEFKLV;
V-33

(SEQ ID NO: 198)

GIVNVPNCNTTKYQQLARTAVAIYNYHEQAHLVFVENLNCKEQLGEGDYY
YITLAATDDAGKKAIYEAKIGVVESAGWTGVEEFKLV;
Q-36

(SEQ ID NO: 199)

GIVNVPNCNTTKYQQLARTAVAIYNYHEQAHLTFVQNLNCKEQLGEGDYY
YITLAATDDAGKKAIYEAKIGVVESAGWTGVEEFKLV;
F-67

(SEQ ID NO: 77)

GIVNVPNCNTTKYQQLARTAVAIYNYHEQAHLTFVENLNCKEQLGEGDYY
YITLAATDDAGKKAIYFAKIGVVESAGWTGVEEFKLV;
L-70

(SEQ ID NO: 79)

GIVNVPNCNTTKYQQLARTAVAIYNYHEQAHLTFVENLNCKEQLGEGDYY
YITLAATDDAGKKAIYEAKLGVVESAGWTGVEEFKLV;
Y-28

(SEQ ID NO: 230)

GIVNVPNCNTTKYQQLARTAVAIYNYHYQAHLTFVENLNCKEQLGEGDYY
YITLAATDDAGKKAIYEAKIGVVESAGWTGVEEFKLV;
R-15

(SEQ ID NO: 231)

GIVNVPNCNTTKYQRLARTAVAIYNYHEQAHLTFVENLNCKEQLGEGDYY
YITLAATDDAGKKAIYEAKIGVVESAGWTGVEEFKLV;
Q-33

(SEQ ID NO: 232)

GIVNVPNCNTTKYQQLARTAVAIYNYHEQAHLQFVENLNCKEQLGEGDYY
YITLAATDDAGKKAIYEAKIGVVESAGWTGVEEFKLV;
R-33

(SEQ ID NO: 233)

GIVNVPNCNTTKYQQLARTAVAIYNYHEQAHLRFVENLNCKEQLGEGDYY
YITLAATDDAGKKAIYEAKIGVVESAGWTGVEEFKLV;
V-21

(SEQ ID NO: 234)

GIVNVPNCNTTKYQQLARTAVAIYNYHEQAHLTFVENLNCKEQLGEGDYY
YITLAATDDAGKKAIYEAKIGVVESAGWTGVEEFKLV;
S-76

(SEQ ID NO: 235)

GIVNVPNCNTTKYQQLARTAVAIYNYHEQAHLTFVENLNCKEQLGEGDYY
YITLAATDDAGKKAIYEAKIGVVESSGWTGVEEFKLV;
W-49

(SEQ ID NO: 236)

GIVNVPNCNTTKYQQLARTAVAIYNYHEQAHLTFVENLNCKEQLGEGDWY
YITLAATDDAGKKAIYEAKIGVVESAGWTGVEEFKLV;
E-28

(SEQ ID NO: 237)

GIVNVPNCNTTKYQQLARTAVAIYNYHEQAHLTFVENLNCKEQLGEGDYY
YITLAATDDAGKKAIYEAKIGVVESAGWTGVEEFKLV;
H-22

(SEQ ID NO: 238)

GIVNVPNCNTTKYQQLARTAVHIYNYHEQAHLTFVENLNCKEQLGEGDYY
YITLAATDDAGKKAIYEAKIGVVESAGWTGVEEFKLV;
K-33

(SEQ ID NO: 239)

GIVNVPNCNTTKYQQLARTAVAIYNYHEQAHLKFVENLNCKEQLGEGDYY
YITLAATDDAGKKAIYEAKIGVVESAGWTGVEEFKLV;
G-17

(SEQ ID NO: 240)

GIVNVPNCNTTKYQQLGRTAVAIYNYHEQAHLTFVENLNCKEQLGEGDYY
YITLAATDDAGKKAIYEAKIGVVESAGWTGVEEFKLV;
T-33

(SEQ ID NO: 241)

GIVNVPNCNTTKYQQLARTAVAIYNYHEQAHLTFVENLNCKEQLGEGDYY
YITLAATDDAGKKAIYEAKIGVVESAGWTGVEEFKLV;
T-19

(SEQ ID NO: 242)

GIVNVPNCNTTKYQQLARTAVAIYNYHEQAHLTFVENLNCKEQLGEGDYY
YITLAATDDAGKKAIYEAKIGVVESAGWTGVEEFKLV;
h2_perm9

(SEQ ID NO: 243)

GIVNVPNCNTTKYQDLAQTAVAIYNYHEQAHLTFVENLNCKEQINGYDYY
YITLAATDDAGKKAIYEAKIGVVESAGWFGVEEFKLV;
gen_perm13

(SEQ ID NO: 244)

GIVNVPNCNTTKYQQLAQTAVAIYNYHEQAHLTFVENLNCKEQLGEGDYY
YITLAATDDAGKKAIYEAKIGVVESAGWTGVEEFKLV;
gen_perm14

(SEQ ID NO: 245)

GIVNVPNCNTTKYQQLAQTAVAIYNYHEQAHLTFVENLNCKEQLGEGDRY
YITLAATDDAGKKAIYEAKIGVVESAGWTGVEEFKLV;
panb_perm9

(SEQ ID NO: 246)

GIVNVPNCNTTKYQQLAQTAVAIYNYHEQAHLTFVENLNCKEQLGGPDRY
YITLAATDDAGKKAIYEAKIGVVEPAGWVAVMEFKLV;
h2_perm2

(SEQ ID NO: 247)

GIVNVPNCNTTKYQDLAQTAVAIYNYHEQAHLTFVENLNCKEQLGEGDYY
YITLAATDDAGKKAIYEAKIGVVESAGWKGVEEFKLV;
pan_perm6

(SEQ ID NO: 248)

GIVNVPNCNTTKYQQLARTAVAIYNYHEQAHLTFVENLNCKEQLGGPDRY
YITLAATDDAGKKAIYEAKIGVVEPAGWVAVEEFKLV;
combs5

(SEQ ID NO: 249)

GIVNVPNCNTTKYQQLARSAVAIYNYHEQAHLTFVENLNCKEQLNDGIYY
YITLAATDDAGKKAIYEAKIGVVESAGWIGVEEFKLV;
pr_gen_pan6

(SEQ ID NO: 250)

GIVNVPNCNTTKYQRLADRAVAIYNYHEQAHLTFVENLNCKEQVGEGVYY
YITLAATDDAGKKAIYEAKIGVVEPAGWVAVEEFKLV;
panb_perm13

(SEQ ID NO: 251)

GIVNVPNCNTTKYQQLAQRAVAIYNYHEQAHLTFVENLNCKEQWGLGIRY
YITLAATDDAGKKAIYEAKIGVVEPAGWVAVMEFKLV;
panb_perm3

(SEQ ID NO: 252)

GIVNVPNCNTTKYQQLARTAVAIYNYHEQAHLTFVENLNCKEQLGGPDRY
YITLAATDDAGKKAIYEAKIGVVEPAGWVAVMEFKLV;
gen_perm29

(SEQ ID NO: 253)

GIVNVPNCNTTKYQQLAQRAVAIYNYHEQAHLTFVENLNCKEQINGYDYY
YITLAATDDAGKKAIYEAKIGVVESAGWTGVEEFKLV;
panb_perm14

(SEQ ID NO: 254)

GIVNVPNCNTTKYQQLAQRAVAIYNYHEQAHLTFVENLNCKEQWGLGIMY
YITLAATDDAGKKAIYEAKIGVVEPAGWVAVMEFKLV;
pan_perm8

(SEQ ID NO: 255)

GIVNVPNCNTTKYQQLAQTAVAIYNYHEQAHLTFVENLNCKEQWGLGIMY
YITLAATDDAGKKAIYEAKIGVVEPAGWVAVEEFKLV;
pan_perm14

(SEQ ID NO: 256)

GIVNVPNCNTTKYQQLAQRAVAIYNYHEQAHLTFVENLNCKEQWGLGIMY
YITLAATDDAGKKAIYEAKIGVVEPAGWVAVEEFKLV;
panb_perm8

(SEQ ID NO: 257)

GIVNVPNCNTTKYQQLAQTAVAIYNYHEQAHLTFVENLNCKEQWGLGIMY
YITLAATDDAGKKAIYEAKIGVVEPAGWAVMEFKLV;
h2_perm1

(SEQ ID NO: 258)

GIVNVPNCNTTKYQDLAQTAVAIYNYHEQAHLTFVENLNCKEQLGEGDYY
YITLAATDDAGKKAIYEAKIGVVESAGWFGVEEFKLV;
gen_perm20

(SEQ ID NO: 259)

GIVNVPNCNTTKYQQLAQDAVAIYNYHEQAHLTFVENLNCKEQLGEGDRY
YITLAATDDAGKKAIYEAKIGVVESAGWTGVEEFKLV;
h2_perm6

(SEQ ID NO: 260)

GIVNVPNCNTTKYQDLAQTAVAIYNYHEQAHLTFVENLNCKEQLGDGVYY
YITLAATDDAGKKAIYEAKIGVVESAGWKGVEEFKLV;
trans_alt3

(SEQ ID NO: 261)

GIVNVPNCNTTKYQQLARSAVAIYNYHEQAHLTFVENLNCKEQVSYGAYY
YITLAATDDAGKKAIYEAKIGVVESAGWVGVEEFKLV;
h2t3

(SEQ ID NO: 262)

GIVNVPNCNTTKYQQLARSAVAIYNYHEQAHLTFVENLNCKEQTAKGNYY
YITLAATDDAGKKAIYEAKIGVVESAGWVGVEEFKLV;
h2_perm5

(SEQ ID NO: 263)

GIVNVPNCNTTKYQDLAQTAVAIYNYHEQAHLTFVENLNCKEQLGDGVYY
YITLAATDDAGKKAIYEAKIGVVESAGWFGVEEFKLV;
singt2

(SEQ ID NO: 264)

GIVNVPNCNTTKYQQLARSAVAIYNYHEQAHLTFVENLNCKEQAGLGGYY
YITLAATDDAGKKAIYEAKIGVVESAGWVAVGEFKLV;
gen_perm25

(SEQ ID NO: 265)

GIVNVPNCNTTKYQQLAQRAVAIYNYHEQAHLTFVENLNCKEQLGEGDYY
YITLAATDDAGKKAIYEAKIGVVESAGWTGVEEFKLV;
hkt3

(SEQ ID NO: 266)

GIVNVPNCNTTKYQQLARSAVAIYNYHEQAHLTFVENLNCKEQVGDGDYY
YITLAATDDAGKKAIYEAKIGVVESAGWVGVNEFKLV;
trans_solo1

(SEQ ID NO: 267)

GIVNVPNCNTTKYQQLARSAVAIYNYHEQAHLTFVENLNCKEQVGGMNYY
YITLAATDDAGKKAIYEAKIGVVESAGWKGVEEFKLV;
pan_perm15

(SEQ ID NO: 268)

GIVNVPNCNTTKYQQLAQRAVAIYNYHEQAHLTFVENLNCKEQLGGPDRY
YITLAATDDAGKKAIYEAKIGVVEPAGWVAVEEFKLV;
gen_perm3

(SEQ ID NO: 269)

GIVNVPNCNTTKYQQLARTAVAIYNYHEQAHLTFVENLNCKEQLGDGVYY
YITLAATDDAGKKAIYEAKIGVVESAGWTGVEEFKLV;
pan_perm7

(SEQ ID NO: 270)

GIVNVPNCNTTKYQQLAQTAVAIYNYHEQAHLTFVENLNCKEQWGLGIRY
YITLAATDDAGKKAIYEAKIGVVEPAGWVAVEEFKLV;
combs17

(SEQ ID NO: 271)

GIVNVPNCNTTKYQQLARRAVAIYNYHEQAHLTFVENLNCKEQAGIGGYY
YITLAATDDAGKKAIYEAKIGVVESAGWVAVGEFKLV;
combs8

(SEQ ID NO: 272)

GIVNVPNCNTTKYQQLARIAVAIYNYHEQAHLTFVENLNCKEQIGEGNYY
YITLAATDDAGKKAIYEAKIGVVESAGWIGVEEFKLV;
pr_gen_pan2

(SEQ ID NO: 273)

GIVNVPNCNTTKYQRLADRAVAIYNYHEQAHLTFVENLNCKEQLGEGDRY
YITLAATDDAGKKAIYEAKIGVVEPAGWVAVEEFKLV;
gen_perm27

(SEQ ID NO: 274)

GIVNVPNCNTTKYQQLAQRAVAIYNYHEQAHLTFVENLNCKEQLGDGVYY
YITLAATDDAGKKAIYEAKIGVVESAGWTGVEEFKLV;
gen_perm4

(SEQ ID NO: 275)

GIVNVPNCNTTKYQQLARTAVAIYNYHEQAHLTFVENLNCKEQLTGYDYY
YITLAATDDAGKKAIYEAKIGVVESAGWTGVEEFKLV;
h2_perm8

(SEQ ID NO: 276)

GIVNVPNCNTTKYQDLAQTAVAIYNYHEQAHLTFVENLNCKEQLTGYDYY
YITLAATDDAGKKAIYEAKIGVVESAGWKGVEEFKLV;
gen_perm28

(SEQ ID NO: 277)

GIVNVPNCNTTKYQQLAQRAVAIYNYHEQAHLTFVENLNCKEQLTGYDYY
YITLAATDDAGKKAIYEAKIGVVESAGWTGVEEFKLV;
vt1

(SEQ ID NO: 278)

GIVNVPNCNTTKYQQLARRAVAIYNYHEQAHLTFVENLNCKEQINGYDYY
YITLAATDDAGKKAIYEAKIGVVESAGWEVVGEFKLV;
pan_perm9

(SEQ ID NO: 279)

GIVNVPNCNTTKYQQLAQTAVAIYNYHEQAHLTFVENLNCKEQLGGPDRY
YITLAATDDACKKAIYEAKIGVVEPAGWVAVEEFKIN;
gen_perm30

(SEQ ID NO: 280)

GIVNVPNCNTTKYQQLAQRAVAIYNYHEQAHLTFVENLNCKEQVGEGVYY
YITLAATDDAGKKAIYEAKIGVVESAGWTGVEEFKLV;
pr_gen_pan3

(SEQ ID NO: 281)

GIVNVPNCNTTKYQRLADRAVAIYNYHEQAHLTFVENLNCKEQLGDGVYY
YITLAATDDAGKKAIYEAKIGVVEPAGWVAVEEFKLV;
panb_perm15

(SEQ ID NO: 282)

GIVNVPNCNTTKYQQLAQRAVAIYNYHEQAHLTFVENLNCKEQLGGPDRY
YITLAATDDAGKKAIYEAKIGVVEPAGWVAVMEFKLV;
pan_perm11

(SEQ ID NO: 283)

GIVNVPNCNTTKYQQLAQDAVAIYNYHEQAHLTFVENLNCKEQWGLGIMY
YITLAATDDAGKKAIYEAKIGVVEPAGWVAVEEFKLV;
combs15

(SEQ ID NO: 284)

GIVNVPNCNTTKYQQLARIAVAIYNYHEQAHLTFVENLNCKEQVSGYDYY
YITLAATDDAGKKAIYEAKIGVVESAGWTGVEEFKLV;
pr_gen_gen2

(SEQ ID NO: 285)

GIVNVPNCNTTKYQRLADRAVAIYNYHEQAHLTFVENLNCKEQLGEGDRY
YITLAATDDAGKKAIYEAKIGVVESAGWTGVEEFKLV;
panb_perm12

(SEQ ID NO: 286)

GIVNVPNCNTTKYQQLAQDAVAIYNYHEQAHLTFVENLNCKEQLGGPDRY
YITLAATDDAGKKAIYEAKIGVVEPAGWVAVMEFKLV;
gen_perm26

(SEQ ID NO: 287)

GIVNVPNCNTTKYQQLAQRAVAIYNYHEQAHLTFVENLNCKEQLGEGDRY
YITLAATDDAGKKAIYEAKIGVVESAGWTGVEEFKLV;
pr_gen_gen6

(SEQ ID NO: 288)

GIVNVPNCNTTKYQRLADRAVAIYNYHEQAHLTFVENLNCKEQVGEGVYY
YITLAATDDAGKKAIYEAKIGVVESAGWTGVEEFKLV;
trans_alt4

(SEQ ID NO: 289)

GIVNVPNCNTTKYQQLARSAVAIYNYHEQAHLTFVENLNCKEQIDYGAYY
YITLAATDDAGKKAIYEAKIGVVESAGWIGVEEFKIN;
h2t1

(SEQ ID NO: 290)

GIVNVPNCNTTKYQQLARIAVAIYNYHEQAHLTFVENLNCKEQLAVGDYY
YITLAATDDAGKKAIYEAKIGVVESAGWIGVEEFKLV;
panb_perm7

(SEQ ID NO: 291)

GIVNVPNCNTTKYQQLAQTAVAIYNYHEQAHLTFVENLNCKEQWGLGIRY
YITLAATDDAGKKAIYEAKIGVVEPAGWVAVMEFKLV;
trans_sclo2

(SEQ ID NO: 292)

GIVNVPNCNTTKYQQLARSAVAIYNYHEQAHLTFVENLNCKEQIAGKTYY
YITLAATDDAGKKAIYEAKIGVVESAGWEGVEEFKLV;
combs12

(SEQ ID NO: 293)

GIVNVPNCNTTKYQQLARSAVAIYNYHEQAHLTEVENLNCKEQSSGYSYY
YITLAATDDAGKKAIYEAKIGVVESAGWEAVTEFKLV;
pan_perm3

(SEQ ID NO: 294)

GIVNVPNCNTTKYQQLARTAVAIYNYHEQAHLTFVENLNCKEQWGLGIRY
YITLAATDDAGKKAIYEAKIGVVEPAGWVAVEEFKLV;
pan_perm13

(SEQ ID NO: 295)

GIVNVPNCNTTKYOQLAQRAVAIYNYHEQAHLTFVENLNCKEQWGLGIRY
YITLAATDDAGKKAIYEAKIGVVEPAGWVAVEEFKLV;
pr_gen_gen4

(SEQ ID NO: 296)

GIVNVPNCNTTKYQRLADRAVAIYNYHEQAHLTFVENLNCKEQLTGYDYY
YITLAATDDAGKKAIYEAKIGVVESAGWTGVEEFKLV;
vt3

(SEQ ID NO: 297)

GIVNVPNCNTTKYQQLARSAVAIYNYHEQAHLTFVENLNCKEQLTGFDYY
YITLAATDDAGKKAIYEAKIGVVESAGWEGVNEFKLV;
combs13

(SEQ ID NO: 298)

GIVNVPNCNTTKYQQLARSAVAIYNYHEQAHLTFVENLNCKEQLGGYSYY
YITLAATDDAGKKAIYEAKIGVVESAGWVGVGEFKLV;
combs24

(SEQ ID NO: 299)

GIVNVPNCNTTKYQQLARSAVAIYNYHEQAHLTFVENLNCKEQINDGVYY
YITLAATDDAGKKAIYEAKIGVVESAGWVGVEEFKLV;
combs3

(SEQ ID NO: 300)

GIVNVPNCNTTKYQQLARSAVAIYNYHEQAHLTFVENLNCKEQSDLGSYY
YITLAATDDAGKKAIYEAKIGVVESAGWIGVEEFKLV;
combs16

(SEQ ID NO: 301)

GIVNVPNCNTTKYQQLARRAVAIYNYHEQAHLTFVENLNCKEQTGLGIYY
YITLAATDDAGKKAIYEAKIGVVESAGWVAVGEFKLV;
h2_perm10

(SEQ ID NO: 302)

GIVNVPNCNTTKYQDLAQTAVAIYNYHEQAHLTFVENLNCKEQINGYDYY
YITLAATDDAGKKAIYEAKIGVVESAGWKGVEEFKLV;
combs4

(SEQ ID NO: 303)

GIVNVPNCNTTKYQQLARSAVAIYNYHEQAHLTFVENLNCKEQTGGPAYY
YITLAATDDAGKKAIYEAKIGVVESAGWIGVEEFKLV;
panb_perm11

(SEQ ID NO: 304)

GIVNVPNCNTTKYQQLAQDAVAIYNYHEQAHLTFVENLNCKEQWGLGIMY
YITLAATDDAGKKAIYEAKIGVVEPAGWVAVMEFKLV;
gen_perm24

(SEQ ID NO: 305)

GIVNVPNCNTTKYQQLAQDAVAIYNYHEQAHLTFVENLNCKEQVGEGVYY
YITLAATDDAGKKAIYEAKIGVVESAGWTGVEEFKLV;
gen_perm5

(SEQ ID NO: 306)

GIVNVPNCNTTKYQQLARTAVAIYNYHEQAHLTFVENLNCKEQINGYDYY
YITLAATDDAGKKAIYEAKIGVVESAGWTGVEEFKLV;
h2t2

(SEQ ID NO: 307)

GIVNVPNCNTTKYQQLARIAVAIYNYHEQAHLTFVENLNCKEQLGSGTYY
YITLAATDDAGKKAIYEAKIGVVESAGWTGVEEFKLV;
pr_gen_pan5

(SEQ ID NO: 308)

GIVNVPNCNTTKYQRLADRAVAIYNYHEQAHLTFVENLNCKEQINGYDYY
YITLAATDDAGKKAIYEAKIGVVEPAGWVAVEEFKLV;
gen_perm16

(SEQ ID NO: 309)

GIVNVPNCNTTKYQQLAQTAVAIYNYHEQAHLTFVENLNCKEQLTGYDYY
YITLAATDDAGKKAIYEAKIGVVESAGWTGVEEFKLV;
gen_perm18

(SEQ ID NO: 310)

GIVNVPNCNTTKYQQLAQTAVAIYNYHEQAHLTFVENLNCKEQVGEGVYY
YITLAATDDAGKKAIYEAKIGVVESAGWTGVEEFKLV;
pan_perm2

(SEQ ID NO: 311)

GIVNVPNCNTTKYQQLARTAVAIYNYHEQAHLTFVENLNCKEQWGLGIMY
YITLAATDDAGKKAIYEAKIGVVEPAGWVAVEEFKLV;
gen_perm15

(SEQ ID NO: 312)

GIVNVPNCNTTKYQQLAQTAVAIYNYHEQAHLTFVENLNCKEQLGDGVYY
YITLAATDDAGKKAIYEAKIGVVESAGWTGVEEFKLV;
gen_perm22

(SEQ ID NO: 313)

GIVNVPNCNTTKYQQLAQDAVAIYNYHEQAHLTFVENLNCKEQLTGYDYY
YITLAATDDAGKKAIYEAKIGVVESAGWTGYEEFKLV;
gen_perm1

(SEQ ID NO: 314)

GIVNVPNCNTTKYQQLARTAVAIYNYHEQAHLTFVENLNCKEQLGEGDYY
YITLAATDDAGKKAIYEAKIGVVESAGWTGVEEFKLV;
panb_perm1

(SEQ ID NO: 315)

GIVNVPNCNTTKYQQLARTAVAIYNYHEQAHLTFVENLNCKEQWGLGIRY
YITLAATDDAGKKAIYEAKIGVVEPAGWVAVMEFKLV;
gen_perm23

(SEQ ID NO: 316)

GIVNVPNCNTTKYQQLAQDAVAIYNYHEQAHLTFVENLNCKEQINGYDYY
YITLAATDDAGKKAIYEAKIGVVESAGWTGVEEFKLV;
pr_gen_gen3

(SEQ ID NO: 317)

GIVNVPNCNTTKYQRLADRAVAIYNYFIEQAHLTFVENLNCKEQLGDGVY
YYITLAATDDAGKKAIYEAKIGVVESAGWTGVEEFKLV;
gen_perm6

(SEQ ID NO: 318)

GIVNVPNCNTTKYQQLARTAVAIYNYHEQAHLTFVENLNCKEQVGEGVYY
YITLAATDDAGKKAIYEAKIGVVESAGWTGVEEFKLV;
gen_perm19

(SEQ ID NO: 319)

GIVNVPNCNTTKYQQLAQDAVAIYNYHEQAHLTFVENLNCKEQLGEGDYY
YITLAATDDAGIKKAIYEAKIGVVESAGWTGVEEFKLV;
combs20

(SEQ ID NO: 320)

GIVNVPNCNTTKYQQLARSAVAIYNYHEQAHLTFVENLNCKEQLGEGDYY
YITLAATDDAGKKAIYEAKIGVVESAGWTGVEEFKLV;
pan_perm10

(SEQ ID NO: 321)

GIVNVPNCNTTKYQQLAQDAVAIYNYHEQAHLTFVENLNCKEQWGLGIRY
YITLAATDDAGKKAIYEAKIGVVEPAGWVAVEEFKLV;
h2_perm7

(SEQ ID NO: 322)

GIVNVPNCNTTKYQDLAQTAVAIYNYHEQAHLTFVENLNCKEQLTGYDYY
YITLAATDDAGKKAIYEAKIGVVESAGWFGVEEFKLV;
pr_gen_pan4

(SEQ ID NO: 323)

GIVNVPNCNTTKYQRLADRAVAIYNYHEQAHLTFVENLNCKEQLTGYDYY
YITLAATDDAGKKAIYEAKIGVVEPAGWVAVEEFKLV;
combs2

(SEQ ID NO: 324)

GIVNVPNCNTTKYQQLARSAVAIYNYHEQAHLTFVENLNCKEQISEGYYY
ITLAATDDAGKKAIYEAKIGVVESAGWIGVEEFKLV;
pan_perm12

(SEQ ID NO: 325)

GIVNVPNCNTTKYQQLAQDAVAIYNYHEQAHLTFVENLNCKEQLGGPDRY
YITLAATDDAGKKAIYEAKIGVVEPAGWVAVEEFKLV;
hkt1

(SEQ ID NO: 326)

GIVNVPNCNTTKYQQLARRAVAIYNYHEQAHLTFVENLNCKEQTNGDVYY
YITLAATDDAGKKAIYEAKIGVVESAGWVGVSEFKLV;
gen_perm2

(SEQ ID NO: 327)

GIVNVPNCNTTKYQQLARTAVAIYNYHEQAHLTFVENLNCKEQLGEGDRY
YITLAATDDAGKKAIYEAKIGVVESAGWTGVEEFKLV;
gen_perm17

(SEQ ID NO: 328)

GIVNVPNCNTTKYQQLAQTAVAIYNYHEQAHLTFVENLNCKEQINGYDVY
YITLAATDDAGKKAIYEAKIGVVESAGWTGVEEFKLV;
panb_perm2

(SEQ ID NO: 329)

GIVNVPNCNTTKYQQLARTAVAIYNYHEQAHLTFVENLNCKEQWGLGIMY
YITLAATDDAGKKAIYEAKIGVVEPAGWVAVMEFKLV;
h2_perm11

(SEQ ID NO: 330)

GIVNVPNCNTTKYQDLAQTAVAIYNYHEQAHLTFVENLNCKEQVGEGVYY
YITLAATDDAGKKAIYEAKIGVVESAGWFGVEEFKLV;
h2_perm4

(SEQ ID NO: 331)

GIVNVPNCNTTKYQDLAQTAVAIYNYHEQAHLTFVENLNCKEQLGEGDRY
Y1TLAATDDAGKKAIYEAKIGVVESAGWKGVEEFKLV;
combs11

(SEQ ID NO: 332)

GIVNVPNCNTTKYQQLARRAVAIYNYHEQAHLTFVENLNCKEQTGFGNYY
YITLAATDDAGKKAIYEAKIGVVESAGWEGVSEFKLV;
panb_perm10

(SEQ ID NO: 333)

GIVNVPNCNTTKYQQLAQDAVAIYNYHEQAHLTFVENLNCKEQWGLGIRY
YITLAATDDAGKKAIYEAKIGVVEPAGWVAVMEFKLV;
combs23

(SEQ ID NO: 334)

GIVNVPNCNTTKYQQLARRAVAIYNYHEQAHLTFVENLNCKEQSGVGVYY
YITLAATDDAGKKAIYEAKIGVVESAGWVAVGEFKLV;
combs10

(SEQ ID NO: 335)

GIVNVPNCNTTKYQQLARRAVAIYNYHEQAHLTFVENLNCKEQIGFGDYY
YITLAATDDAGKKAIYEAKIGVVESAGWEGVSEFKLV;
pr_gen_gen1

(SEQ ID NO: 336)

GIVNVPNCNTTKYQRLADRAVAIYNYHEQAHLTFVENLNCKEQLGEGDYY
YITLAATDDAGKKAIYEAKIGVVESAGWTGVEEFKLV;
combs18

(SEQ ID NO: 337)

GIVNVPNCNTTKYQQLARIAVAIYNYHEQAHLTFVENLNCKEQSGLGDYY
YITLAATDDAGKKAIYEAKIGVVESAGWTGVEEFKLV;
hkt2

(SEQ ID NO: 338)

GIVNVPNCNTTKYQQLARSAVAIYNYHEQAHLTFVENLNCKEQIGDGTYY
YITLAATDDAGKKAIYEAKIGVVESAGWEVVSEFKLV;
h2_perm12

(SEQ ID NO: 339)

GIVNVPNCNTTKYQDLAQTAVAIYNYHEQAHLTFVENLNCKEQVGEGVYY
YITLAATDDAGKKAIYEAKIGVVESAGWKGVEEFKLV;
pr_gen_pan1

(SEQ ID NO: 340)

GIVNVPNCNTTKYQRLADRAVAIYNYHEQAHLTFVENLNCKEQLGEGDYY
YITLAATDDAGKKAIYEAKIGVVEPAGWVAVEEFKLV;
singt1

(SEQ ID NO: 341)

GIVNVPNCNTTKYQQLARRAVAIYNYHEQAHLTEVENLNCKEQTAGNIGY
YYITLAATDDAGKKAIYEAKIGVVESAGWVAVGEFKLV;
combs1

(SEQ ID NO: 342)

GIVNVPNCNTTKYQQLARIAVAIYNYHEQAHLTFVENLNCKEQISDGVYY
YLILAATDDAGKKAIYEAKIGVVESAGWTGVEEFKLV;
gen_perm21

(SEQ ID NO: 343)

GIVNVPNCNTTKYQQLAQDAVAIYNYHEQAHLTFVENLNCKEQLGDGVYY
YITLAATDDAGKKAIYEAKIGVVESAGWTGVEEFKLV;
h2_perm3

(SEQ ID NO: 344)

GIVNVPNCNTTKYQDLAQTAVAIYNYHEQAHLTFVENLNCKEQLGEGDRY
YITLAATDDAGKKAIYEAKIGVVESAGWFGVEEFKLV;
combs14

(SEQ ID NO: 345)

GIVNVPNCNTTKYQQLARRAVAIYNYHEQAHLTFVENLNCKEQLTGYDYY
YITLAATDDAGKKAIYEAKIGVVESAGWEVVAEFKLV;
and
pr_gen_gen5

(SEQ ID NO: 346)

GIVNVPNCNTTKYQRLADRAVAIYNYHEQAHLTFVENLNCKEQINGYDYY
YITLAATDDAGKKAIYEAKIGVVESAGWTGVEEFKLV.

In another embodiment, the polypeptide RBS inhibitors are at least 70% identical over the full length of the amino acid sequence in Table 4, wherein the polypeptide does not comprise the amino acid sequence of MINMKVAISMDVDKISNSFEDCKYFLIVRIDDNEVKSTKVIFNDESGKKSIVKE NVNAIICKNISEENYKKFSKKIEWHAEGDDVDKNISLEIEGELSKISNP (SEQ ID NO: 130).

TABLE 4
SB24 Genus (SEQ ID NO: 4)

| Residue | AAs |
|---|---|
| 1 | G, A |
| 2 | I, S, T |
| 3 | G |
| 4 | M |
| 5 | V, L |
| 6 | A |
| 7 | I |
| 8 | S |
| 9 | M |
| 10 | D |
| 11 | T |
| 12 | D |
| 13 | K |
| 14 | I |
| 15 | S |
| 16 | N |
| 17 | S |
| 18 | F |
| 19 | E |
| 20 | D |
| 21 | C |
| 22 | K |
| 23 | Y |
| 24 | F |
| 25 | Any amino acid |
| 26 | M, L, F, I, E |
| 27 | Any amino acid |
| 28 | V, M, I, L |
| 29 | V, A, S, P, E, D, Q, N, V, T, K |
| 30 | S |
| 31 | A |
| 32 | G |
| 33 | W, G |
| 34 | T, I, V, D, E |
| 35 | N |
| 36 | T |
| 37 | I |
| 38 | F, I, L, T, S |
| 39 | N |
| 40 | D |
| 41 | E |
| 42 | S |
| 43 | G |
| 44 | K |
| 45 | K, E |
| 46 | E |
| 47 | I, L |
| 48 | V |
| 49 | K, E, M, N |
| 50 | M |
| 51 | N, D, E, S, G |
| 52 | V |
| 53 | D |
| 54 | A |
| 55 | I |
| 56 | I |
| 57 | C |
| 58 | K |
| 59 | N |
| 60 | I |
| 61 | S |
| 62 | E |
| 63 | E |
| 64 | N |
| 65 | Y |
| 66 | K |
| 67 | K, E, R |
| 68 | F |
| 69 | S |
| 70 | K |
| 71 | K, E |
| 72 | I |
| 73 | E |
| 74 | I |
| 75 | Y |
| 76 | H |
| 77 | A |
| 78 | E |
| 79 | G |
| 80 | D |
| 81 | D |
| 82 | V |
| 83 | D, N, K, E |
| 84 | K |
| 85 | N |
| 86 | I |
| 87 | S |
| 88 | L |
| 89 | F, I, L |
| 90 | I, L, M, T, V, A |
| 91 | E, D, K, R, G |
| 92 | G |
| 93 | E |
| 94 | L |
| 95 | S |
| 96 | K |
| 97 | I |
| 98 | S |
| 99 | N |
| 100 | P |

As disclosed in detail in the examples that follow, numerous HA-binding polypeptides of this embodiment have been identified, and the inventors have discovered that residues 1-3, 25-38, 45-51, and position 83 primarily make up the interface for HA protein binding; these regions have been subjected to extensive further analysis to identify variability within these regions. The remaining residues (4-24, 39-44, 52-82, and 84 to 100) can be modified, as these residues are not involved in the HA protein interface. Such modifications may comprise, for example, conservative amino acid substitutions. Thus, in one preferred embodiment, the polypeptide RBS inhibitors are at least 70% identical with the amino acid sequence of SEQ ID NO:4, wherein variability is within residues 4-24, 39-44, 52-82, and 84 to 100. in various embodiments, the polypeptide RBS inhibitors are at least 75%, 80%, 85%, 90%, 91%, 92%, 93%, 94%, 95%, 96%, 97%, 98%, 99%, or completely identical with to the amino acid sequence of SEQ ID NO:4 (Table 4), over its full length. In various preferred embodiments, the polypeptide RBS inhibitors are at least 75%, 80%, 85%, 90%, 91%, 92%, 93%, 94%, 95%, 96%, 97%, 98%, 99%, or completely identical with the amino acid sequence of SEQ ID NO:4, wherein variability is within residues 4-24, 39-44, 52-82, and 84 to 100.

In one embodiment, the polypeptide RBS inhibitors comprise or consist of a polypeptide at least 70% identical to the amino acid sequence in Table 5,

TABLE 5
(SEQ ID NO: 5)

| Residue | AAs |
|---|---|
| 1 | G, A |
| 2 | I, S, T |
| 3 | G |
| 4 | M |
| 5 | V, L |
| 6 | A |
| 7 | I |
| 8 | S |
| 9 | M |
| 10 | D |
| 11 | T |
| 12 | D |
| 13 | K |
| 14 | I |
| 15 | S |
| 16 | N |
| 17 | S |
| 18 | F |
| 19 | E |
| 20 | D |
| 21 | C |
| 22 | K |
| 23 | Y |
| 24 | F |
| 25 | L, Q |
| 26 | L, I, E |
| 27 | M, V, E, Y |
| 28 | V, M, L |
| 29 | V, P, E, K, L, A |
| 30 | S |
| 31 | A |
| 32 | G |
| 33 | W |
| 34 | T |
| 35 | N |
| 36 | T |
| 37 | I |
| 38 | F, S |
| 39 | N |
| 40 | D |
| 41 | E |
| 42 | S |
| 43 | G |
| 44 | K |
| 45 | K, E |
| 46 | E |
| 47 | I |
| 48 | V |
| 49 | K, E |
| 50 | M |
| 51 | N, G, S |
| 52 | V |
| 53 | D |
| 54 | A |
| 55 | I |
| 56 | I |
| 57 | C |
| 58 | K |
| 59 | N |
| 60 | I |
| 61 | S |
| 62 | E |
| 63 | E |
| 64 | N |
| 65 | Y |
| 66 | K |
| 67 | K |
| 68 | F |
| 69 | S |
| 70 | K |
| 71 | K |
| 72 | I |
| 73 | E |
| 74 | I |
| 75 | Y |
| 76 | H |
| 77 | A |
| 78 | E |
| 79 | G |
| 80 | D |
| 81 | D |
| 82 | V |
| 83 | D, N, K, E |
| 84 | K |
| 85 | N |
| 86 | I |
| 87 | S |
| 88 | L |
| 89 | F |
| 90 | I, L, A |
| 91 | E |
| 92 | G |
| 93 | E |
| 94 | L |
| 95 | S |
| 96 | K |
| 97 | I |
| 98 | S |
| 99 | N |
| 100 | P |

In one preferred embodiment, the polypeptide RBS inhibitors are at least 70% identical with the amino acid sequence of SEQ ID NO:5, wherein variability is within residues 4-24, 39-44, 52-82, and 84 to 100. In various embodiments, the polypeptide RBS inhibitors of the invention are at least 75%, 80%, 85%, 90%, 91%, 92%, 93%, 94%, 95%, 96%, 97%, 98%, 99%, or completely identical with to the amino acid sequence of SEQ ID NO:5 over its full length. In various preferred embodiments, the polypeptide RBS inhibitors are at least 75%, 80%, 85%, 90%, 91%, 92%, 93%, 94%, 95%, 96%, 97%, 98%, 99%, or completely identical with the amino acid sequence of SEQ ID NO:5, wherein variability is within residues 1-24, 39-44, 52-82, and 84 to 100.

In various further embodiments, the polypeptide RBS inhibitors comprise or consist of a polypeptide with an amino acid sequence selected from the group consisting of:

SB24

(SEQ ID NO: 40)

GIGMVAISMDTDKISNSFEDCKYFLIVVVSAGWTNTIFNDESGKKEIVKM
NVDAIICKNISEENYKKFSKKIEIYHAEGDDVDKNISLFIEGELSKISN
P;
Variant H3 24-2 (V27M, F38S, K49E)

(SEQ ID NO: 41)

GIGMVAISMDTDKISNSFEDCKYFLIMVVSAGWTNTISNDESGKKEIVEM
NVDAIICKNISEENYKKFSKKIEIYHAEGDDVDKNISLFIEGELSKISN
P;
Variant H3 24-3a (I2T, V29E)

(SEQ ID NO: 42)

GIGMVAISMDTDKISNSFEDCKYFLIVVESAGWTNTIFNDESGKKEIVKM
NVDAIICKNISEENYKKFSKKIEIYHAEGDDVDKNISLFIEGELSKISN
P;
Variant H3 24-3b (V28M)

(SEQ ID NO: 43)

GTGMVAISMDTDKISNSFEDCKYFLIVMVSAGWTNTIFNDESGKKEIVKM
NVDAIICKNISEENYKKFSKKIEIYHAEGDDVDKNISLFIEGELSKISN
P;
Variant H3 24-8 (V27M, K49E)

(SEQ ID NO: 44)

GTGMVAISMDTDKISNSFEDCKYFLIMVVSAGWTNTIFNDESGKKEIVEM
NVDAIICKNISEENYKKFSKKIEIYHAEGDDVDKNISLFIEGELSKISN
P;
SB24solo2

(SEQ ID NO: 45)

ASGMLAISMDTDKISNSFEDCKYFLIELKSAGWTNTIFNDESGKEEIVKM
GVDAIICKNISEENYKKFSKKIEIYHAEGDDVNKNISLFLEGELSKISN
P;
SB24solo6

(SEQ ID NO: 46)

ASGMLAISMDTDKISNSFEDCKYFQLELPSAGWTNTIFNDESGKEEIVKM
NVDAIICKNISEENYKKFSKKIEIYHAEGDDVKKNISLFLEGELSKISN
P;
SB24NC1

(SEQ ID NO: 47)

ASGMLAISMDTDKISNSFEDCKYFQIELPSAGGTNTIFNDESGKEEIVKM
GVDAIICKNISEENYKKFSKKIEIYHAEGDDVKKNISLFIEGELSKISN
P;
SB24alt1-1

(SEQ ID NO: 125)

ASGMLAISMDTDKISNSFEDCKYFQLYVLSAGWTNTIFNDESGKEEIVKM
SVDAIICKNISEENYKKFSKKIEIYHAEGDDVDKNISLFLEGELSKISN
P;
SB24SingNC1

(SEQ ID NO: 127)

ASGMLAISMDTDKISNSFEDCKYFQIELPSAGWTNTIFNDESGKEEIVKM
GVDAIICKNISEENYKKFSKKIEIYHAEGDDVEKNISLFLEGELSKISN
P;
and
SB24SingNC3

(SEQ ID NO: 128)

ASGMLAISMDTDKISNSFEDCKYFQLVLASAGWTNTIFNDESGKEEIVKM
GVDAIICKNISEENYKKFSKKIEIYHAEGDDVDKNISLFAEGELSKISN
P.

In a further embodiment, the polypeptide RBS inhibitors comprise or consist of a polypeptide of the amino acid sequence in Table 6:

TABLE 6
Disulfide genus (SEQ ID NO: 6)

| Residue # | Amino acids |
|---|---|
| 1 | G |
| 2 | Any amino acid |
| 3 | C |
| 4 | Any amino acid |
| 5 | Any amino acid |
| 6 | V, M |
| 7 | P, A, T, I, L, V |
| 8 | S |
| 9 | A |
| 10 | G |
| 11 | W |
| 12 | E, V, T, I, A, K |
| 13 | Any amino acid |
| 14 | C |
| 15 | Any amino acid or is |
| | absent |

The polypeptide RBS inhibitors of this embodiment can assume a hairpin-like structure that can be stabilized through a disulfide bridge by the cysteine residues at a rron-hydrogen bonding beta strand pair. In one embodiment, e polypeptide RBS ibitors comprise or consist of the amino acid sequence in Table 7:

TABLE 7
(SEQ ID NO: 7)

| Residue # | Amino acids |
|---|---|
| 1 | G |
| 2 | G, W, R, H, D, Y, E, Q, C |
| 3 | C |
| 4 | I, L, M, F |
| 5 | G |
| 6 | V |
| 7 | P, A |
| 8 | S |
| 9 | A |
| 10 | G |
| 11 | W |
| 12 | E, V, T, I, A, K |
| 13 | W, I, V, R, M, G, L, V |
| 14 | C |
| 15 | P, W, D, R, S, A, E, Y, H, |
| | Q, C, G |

In a further embodiment, the polypeptide RBS inhibitors comprise or consist of a polypeptide of the amino acid sequence in Table 8:

TABLE 8
(SEQ ID NO: 8)

| Residue # | Amino acids |
|---|---|
| 1 | G |
| 2 | G, W, R, H, |
| 3 | C |
| 4 | I, L, M |
| 5 | G |
| 6 | V |
| 7 | P, A |
| 8 | S |
| 9 | A |
| 10 | G |
| 11 | W |
| 12 | E, V, T, I |
| 13 | W, I, V, R |
| 14 | C |
| 15 | P, W, D, R, |

In various further embodiments, the polypeptide RBS inhibitors comprise or consist of a polypeptide selected from the group consisting of:

(SEQ ID NO: 48)

GXCIGVPSAGWEXCW;

(SEQ ID NO: 49)

GGCIGVPSAGWEWCP;

(SEQ ID NO: 50)

GGCLGVPSAGWEICW;

(SEQ ID NO: 51)

GWCIGVPSAGWEICW;

(SEQ ID NO: 52)

GRCIGVPSAGWEVCW;

(SEQ ID NO: 53)

GHCMGVASAGWEICW;
and

(SEQ ID NO: 54)

GDCIGVASAGWEWCP.

In a further embodiment, the polypeptide RBS inhibitors comp ise car consist of a polypeptide of the amino acid sequence in Table 9.

TABLE 9
(SEQ ID NO: 9)

| Residue | Options |
|---|---|
| 1 | G |
| 2 | G, W, R, H, D, Y |
| 3 | C |
| 4 | I, L, M |
| 5 | G |
| 6 | V |
| 7 | P, A |
| 8 | S |
| 9 | A |
| 10 | G |
| 11 | W |
| 12 | E |
| 13 | W, I, V |
| 14 | C |
| 15 | W, P |

In various further embodiments, the polypeptide RBS inhibitors comprise or consist of a polypeptide selected from the group consisting of:

(SEQ ID NO: 55)

GGCIGVPSAGWEWCP;

(SEQ ID NO: 56)

GGCLGVPSAGWEWCP;

(SEQ ID NO: 57)

GWCIGVPSAGWEICW;

(SEQ ID NO: 58)

GWCIGVPSAGWEICW;

(SEQ ID NO: 59)

GRCIGVPSAGWEVCW;

(SEQ ID NO: 60)

GHCMGVASAGWEICW;

(SEQ ID NO: 61)

GDCIGVASAGWEWCP;

(SEQ ID NO: 62)

GYCIGVPSAGWEVCW;

(SEQ ID NO: 63)

GYCIGVPSAGWEICW;
and

(SEQ ID NO: 64)

GWCMGVPSAGWEICW.

In a further embodiment, the polypeptide RBS inhibitors comprise or consist of a polypeptide of the amino acid sequence in Table 10.

TABLE 10
(SEQ ID NO: 10)

| Residue | Options |
|---|---|
| 1 | G |
| 2 | S, F, D, C, M, N, K, G, T, |
| | R, W, V |
| 3 | C |
| 4 | P, C, T, G, F, R, A, Y, I, |
| | W, V |
| 5 | F, L, C, Y, H, A, W, R, V, |
| | P, G, K |
| 6 | V, M, A |
| 7 | T, I, L, V, A |
| 8 | S |
| 9 | A |
| 10 | G |
| 11 | W |
| 12 | E |
| 13 | K, E, P, M, V, I, T, A, G |
| 14 | C |
| 15 | L, Y, E, S, K, A, N, R, V, |
| | or is absent |

In various further embodiments, the polypeptide RBS inhibitors comprise or consist of a polypeptide selected from the group consisting of:

(SEQ ID NO: 65)

GSCYRVVSAGWETC;

(SEQ ID NO: 65)

GSCYRVVSAGWETC;

(SEQ ID NO: 67)

GGCARVASAGWEICN;

(SEQ ID NO: 68)

GKCRWVASAGWEVCA;

(SEQ ID NO: 69)

GNCFAVVSAGWEKCK;

(SEQ ID NO: 70)

GMCTHVLSAGWEPCL;

(SEQ ID NO: 71)

GMCTHVLSAGWEPCL;

(SEQ ID NO: 72)

GCCGYVISAGWEMCS;

(SEQ ID NO: 73)

GDCTCMISAGWEPCE;

(SEQ ID NO: 74)

GFCCLVTSAGWEECY;

(SEQ ID NO: 75)

GFCCLVTSAGWEECY;

(SEQ ID NO: 76)

GSCPFVTSAGWEKCL;

(SEQ ID NO: 78)

GDCIVVASAGWEACR_;

(SEQ ID NO: 80)

GRCICALSAGWETCA;

(SEQ ID NO: 81)

GWCWGVISAGWEGCR;
and

(SEQ ID NO: 82)

GVCVKVASAGWEECV.

In a further embodiment, the polypeptide RBS inhibitors comprise or consist of a polypeptide selected from the group consisting of the following, each of which is shown in the examples that follow to strongly bind the HA protein:

SB53

(SEQ ID NO: 85)

GIVNVPNPNNTKFQQLARNAIQNYNDNQNAHLEFVENLNVKEQVTGGIMY
YITLAATDDAGKKKIYKAKIAVVDSAGWEGITEFKLV;
SB55

(SEQ ID NO: 86)

GIVNVPNPNNTKFQQLARSAIQNYNDNQNAHLEFVENLNVKEQVTGGIMY
YITLAATDDAGKKKIYKAKIAVVDSAGWEGITEFKLV;
SB41

(SEQ ID NO: 87)

KEITNALETWGALGQDINLDIPSFQMSDDIADIKWEKTQDNKMIVVFSAG
WIAKDTYDLYENGTLKIAHLTTDDQAIYKVSITDTKGKNVLEKIFDLKIQ
ERVSKPKISWTCINTTLTCEVMNGTDPELNLYQDGKHLKLSQRVITHKWT
TSLSAKFKCTAGNKVSKESSVEPVSCPEK
SB60

(SEQ ID NO: 88)

KIIITGEPGVGKTTLVKKIVERLGKRAIGFWTEEVTDPETKKRTGFRIIT
TEGKKKVFSVVSAGWESKQNFEELAIPILERAYREAKKDRRKVIIIDEIG
DALGSSKFRDLVRQIHDPNVNVVATIPIRDDAPLIKEIRRLPGAVLIELT
PENRDVILEDILSLLER;
and
SB23

(SEQ ID NO: 89)

GHPTLKTPESVTGTWKGDVKIQCIYDPLRGYEQTEVKWLVRHGSDSVTIF
ERVSSAGWDGISQDKYNGRLQVSDSVPGDVSLQINTLQMDDRNHYTCEVT
WQTPDGNQVIRDKIIELRVRK.

In one embodiment, the isolated polypeptide+polypeptide RBS inhibitor (HA binding protein) comprises a combination according to the following (which may further comprise a linker C terminal to the isolated polypeptide, and N terminal to the polypeptide RBS inhibitor):

(1) isolated polypeptide selected from the group consisting of SEQ NOS: 200-204, 206, 208-212, + polypeptide RBS inhibitor selected from the group consisting of SEQ ID NOS: 1-3, 11-39, 77, 79, 95-97 126, 131-135-199 and SEQ ID NOS: 228-346; or

(2) isolated polypeptide selected from the group consisting of SEQ ID NOS: 205 or 207+ polypeptide RBS inhibitor selected from the group consisting of SEQ ID NOS: 4-5, 40-47, 125, and 127-128.

As shown in the examples that follow, the polypeptides of the invention can, for example, self-assemble into multimers (dimers, trimers, pentamers, hexamers, etc.) Thus, in another embodiment the isolated polypeptide H- polypeptide RBS inhibitor (HA binding protein) comprises a multimer of polypeptide units (dimer, trimer, pentamer, hexamer, etc.). In one embodiment, the multimer comprises a. multimer of identical polypeptides of the invention and identical polypeptide RBS inhibitors; in another embodiment, the multimer comprises a multimer of identical polypeptides of the invention and two or more different polypeptide RBS inhibitors; in another embodiment, the multimer may comprise different polypeptides of the invention and either identical or different polypeptide RBS inhibitors.

As used throughout the present application, the term “polypeptide” is used in its broadest sense to refer to a sequence of subunit amino acids. The polypeptides of the invention may comprise L-amino acids, D-amino acids (which are resistant to L-amino acid-specific proteases in vivo), or a combination of D- and L-amino acids. The polypeptides described herein may be chemically synthesized or recombinantly expressed. The polypeptides may be linked to other compounds to promote an increased half-life in vivo, such as by PEGylation, HESylation, PASylation, glycosylation, etc. Such linkage can be covalent or non-covalent as is understood by those of skill in the art.

In another embodiment that can be combined with any embodiment or combination of embodiments herein, the polypeptides of the invention further comprise a tag. The tag can be present on the polypeptide of the invention (self-assembling polypeptide) and/or the polypeptide RBS inhibitor (when present). In one embodiment, the tag is present on the RBS inhibitor. In exemplary embodiments, the tag may comprise a detectable moiety, a therapeutic agent, a toxin, a binding protein (for example, to other flu strains or to recruit immune cells; or an albumin binding protein, an Fe, or any other moiety to increase serum half-life), or a moiety to facilitate purification. The tag(s) can be linked to the polypeptide through covalent bonding, including, but not limited to, disulfide bonding, hydrogen bonding, electrostatic bonding, recombinant fusion and conformational bonding. Alternatively the tag(s) can be linked to the polypeptide by means of one or more linking compounds. Techniques for conjugating tags to polypeptides are well known to the skilled artisan. Polypeptides comprising a detectable tag can be used diagnostically to, for example, assess if a subject has been infected with influenza virus or monitor the development or progression of an influenza virus infection as part of a clinical testing procedure to, e.g, determine the efficacy of a given treatment regimen. However, they may also be used for other detection and/or analytical and/or diagnostic purposes. Any suitable detection tag can be used, including but not limited to enzymes, prosthetic groups, fluorescent materials, luminescent materials, bioluminescent materials, radioactive materials, positron emitting metals, and non radioactive paramagnetic metal ions. The tag used will depend on the specific detectionianalysis/diagnosis techniques and/or methods used such as immunohistochemical staining of (tissue) samples, flow cytometric detection, scanning laser cytometric detection, fluorescent immunoassays, enzyme-linked immunosorbent assays (ELISAs), radioimmunoassays (RIAs), bioassays (e.g., neutralization assays), Western blotting applications, etc. For mimunohistochemical staining of tissue samples preferred tags are enzymes that catalyze production and local deposition of a detectable product. Enzymes typically conjugated to polypeptides to permit their immunohistochemical visualization are well known and include, but are not limited to, acetylcholinesterase, alkaline phosphatase, beta-galactosidase, glucose oxidase, horseradish peroxidase, and urease. Typical substrates for production and deposition of visually detectable products are also well known to the skilled person in the art. The polypeptides can be labeled using colloidal gold or they can be labeled with radioisotopes.

When the polypeptides with an RBS inhibitor are used for flow cytometric detections, scanning laser cytometric detections, or fluorescent immunoassays, the tag may comprise, for example, a fluorophore. A wide variety of fluorophores useful for fluorescently labeling the polypeptides of the invention are known to the skilled artisan. When the polypeptides are used for in vivo diagnostic use, the tag can comprise, for example, magnetic resonance imaging (MRI) contrast agents, such as gadolinium diethylenetriaminepentaacetic acid, to ultrasound contrast agents or to X-ray contrast agents, or by radioisotopic labeling.

The polypeptides with an RBS inhibitor can also be attached to solid supports, which are particularly useful for in vitro assays or purification of influenza virus or HA protein. Such solid supports might be porous or nonporous, planar or nonplanar and include, but are not limited to, glass, cellulose, polyacrylamide, nylon, polystyrene, polyvinyl chloride or polypropylene supports. The polypeptides can also, for example, usefully be conjugated to filtration media, such as NHS-activated Sepharose or CNBr-activated Sepharose for purposes of affinity chromatography. They can also usefully be attached to paramagnetic microspheres, typically by biotin-streptavidin interaction. The microspheres can be used for isolation of influenza virus or HA protein from a sample containing influenza virus or HA protein. As another example, the polypeptides of the invention can usefully be attached to the surface of a microtiter plate for ELISA.

The polypeptides of the invention (with or without polypeptide RBS inhibitors) can be fused to marker sequences to facilitate purification. Examples include, but are not limited to, the hexa-histidine tag, the myc tag or the flag tag.

The polypeptides of the invention with an RBS inhibitor can be conjugated to an antigen recognized by the immune system of a subject to which the polypeptide is administered. Conjugation methods for attaching the antigens and polypeptide are well known in the art and include, but are not limited to, the use of cross-linking agents. The polypeptide will bind to the influenza virus HA protein and the antigen will initiate a T-cell attack on the conjugate that will facilitate destruction of the influenza virus.

In another aspect, the present invention provides novel RBS inhibitors, wherein the inhibitor comprises or consists of a polypeptide having the amino acid sequence of SEQ ID NO:1,

TABLE 11
SB52 genus (SEQ ID NO: 1)

| Residue | AAs |
|---|---|
| 1 | G, D, S, K, Q |
| 2 | I |
| 3 | V |
| 4 | N |
| 5 | V |
| 6 | P |
| 7 | N, K |
| 8 | P, C |
| 9 | N, K |
| 10 | N, T |
| 11 | T, K, P |
| 12 | K |
| 13 | F, Y |
| 14 | Q, R, I |
| 15 | Q, K, R |
| 16 | L |
| 17 | A, G |
| 18 | R, N, Y, T, S |
| 19 | N, R, S, Y, T, Q |
| 20 | A |
| 21 | I, V |
| 22 | A, S, Y, H |
| 23 | N, I, V |
| 24 | Y, V, W, M, I |
| 25 | N |
| 26 | D, Y, N, H |
| 27 | N, H, Y, Q, D |
| 28 | Q, K, E, D, W, Y |
| 29 | N, P, E, D |
| 30 | A, E |
| 31 | H |
| 32 | L |
| 33 | E, T, R, K, Q, S, Y, A, |
| | L, H, F, M, V, I, N |
| 34 | F |
| 35 | V |
| 36 | E, H, Q |
| 37 | N |
| 38 | L, M |
| 39 | N |
| 40 | V, C |
| 41 | K |
| 42 | E, D, K |
| 43 | Q, K |
| 44 | Any amino acid, or |
| | absent |
| 45 | Any amino acid, or |
| | absent |
| 46 | G (Position 46 and 47 |
| | are set so that one of |
| | them is glycine and |
| | the other is any of the |
| | following amino |
| | acids; |
| | N, H, D, S, K, Y, H, A, V, T, |
| | I, Q, D, E, M, L, F), or |
| | absent |
| 47 | G (Position 46 and 47 |
| | are set so that one of |
| | them is glycine and |
| | the other is any of the |
| | following amino |
| | acids; |
| | N, H, D, S, K, Y, H, A, V, T, |
| | I, Q, D, E, M, L, F, K) |
| 48 | Any amino acid |
| 49 | M, L, Y, W |
| 50 | Y |
| 51 | Y, W |
| 52 | I |
| 53 | T |
| 54 | L |
| 55 | A |
| 56 | A |
| 57 | T |
| 58 | D |
| 59 | D, Q |
| 60 | A |
| 61 | G |
| 62 | K, E |
| 63 | K |
| 64 | K, S, A, W, L, M, Q, |
| | G |
| 65 | I |
| 66 | Y |
| 67 | K, S, A, Y, W, M, F |
| 68 | A, K, Y, T |
| 69 | K |
| 70 | I, V, L |
| 71 | A, G |
| 72 | V |
| 73 | V or is absent |
| 74 | D, E |
| 75 | S |
| 76 | A, S |
| 77 | G |
| 78 | W |
| 79 | E, V, I, K, A, T, L |
| 80 | G, V, A |
| 81 | V, L |
| 82 | Any amino acid |
| 83 | E |
| 84 | F |
| 85 | K, A, E, D |
| 86 | L |
| 87 | V, I, M |

wherein one or more of the following is true:

(a) 1, 2, or all 3 of amino acids 44-46 are absen;

(b) amino acid 11 is K or P;

(c) amino acid 14 is 1 or R;

(d) amino acid 15 is K or R;

(e) amino acid 17 is G;

(f) amino acid 19 is Q or T;

(g) amino acid 22 is EL S, or Y;

(h) amino acid 24 is 1, M, V, or W;

(i) amino acid 27 is D;

(j) amino acid 28 is D, E, K, W, or

(k) amino acid 29 is D, E, or P;

(l) amino acid 30 is E;

(m) amino acid 33 is A, F, H, I, K, L, M, N, Q, R, S, T, or Y

(n) amino acid 36 is H or Q;

(o) amino acid 43 is K

(p) amino acid 47 is K;

(q) amino acid 49 is L, W, or Y;

(r) amino acid 51 is W;

(s) amino acid 59 is Q;

(t) amino acid 62 is E;

(u) amino acid 64 is A, G, L. M Q. S. or W;

(v) amino acid 67 is A, F, M, S, W, or Y;

(w) amino acid 68 is T;

(x) amino acid 70 is L;

(y) amino acid 76 is 5;

(z) amino acid 79 is L;

(aa) amino acid 81 is L;

(bb) amino acid 85 is A, D, or E; and/or

(cc) amino acid 87 is I or M

As shown in the examples that follow, peptides g n the recited genus are novel and improved RBS inhibitors.

Variants were selected independently at 22° C., 30° C., 37° C. and 42° C. for better binding to hemagglutinin. Variants that showed increasing enrichment across these temperature ranges were identified as substitutions that improved binding even at higher temperatures.

In one embodiment the novel RBS inhibitors, 1, 2, or all 3 of amino acids 44-46 are absent; in a particular embodiment, all 3 of amino acids 44-46 are absent; this embodiment is a particularly strong binder when used in combination with the polypeptide multimers of the invention to appropriately display the RBS inhibitor in alignment with the RBS of the trimeric hemagglutinin of influenza.

In a further embodiment, the novel RBS inhibitor peptide comprises or consists of the amino acid sequence of a polypeptide selected from the group consisting of SEQ ID NOS: 77, 79, 135-199, and 228-346. In one particular embodiment, the novel RBS inhibitor peptide comprises or consists of the amino acid sequence of a polypeptide selected from the group consisting of SEQ ID NO:135 and 141.

In a further aspect, the present invention provides pharmaceutical compositions, comprising one or more polypeptides of the invention that include an RBS inhibitor; or a novel RBS inhibitor of the invention, and a pharmaceutically acceptable carrier. In this embodiment, the polypeptides of the invention may be used, for example, in any of the methods of the present invention. The pharmaceutical composition may comprise in addition to the polypeptide of the invention (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, henzethonium, chiorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides may be the sole active agent in the pharmaceutical composition, or the composition may further comprise one or more other active agents suitable for an intended use, including but not limited to anti-HA and anti-NA antibodies.

In a further aspect, the present invention provides isolated nucleic acids encoding a polypeptide or RBS inhibitor of the present invention. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present invention provides recombinant expression vectors comprising the isolated nucleic acid of the invention operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Amhion 1998 Catalog (Ambion, Austin, Tex.). The expression vector must be replicable in the host organisms either as an episome or by integration into host chromosomal DNA. In a preferred embodiment, the expression vector comprises a plasmid. However, the invention is intended to include other expression vectors that serve equivalent functions, such as viral vectors.

In a still further aspect, the present invention provides host cells that have been transfected with the recombinant expression vectors disclosed herein, wherein the host cells can be either prokaryotic (such as bacteria) or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, N.Y.).

A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide.

In a further aspect, the present invention provides assemblies, comprising:

(a) a scaffold and

(b) three hemagglutinin (HA) receptor binding site (RBS) inhibitors bound to the scaffold, wherein the scaffold organizes the three inhibitors such that each inhibitor is between about 40 A° and about 60 A° in distance from each other and at an angle of between about 57 degrees and about 63 degrees from each other.

The inventors have discovered an appropriate organizational system by which three HA RBS inhibitors can be arranged on a scaffold to greatly increase HA affinity, thus providing greatly enhanced performance in diagnostic and therapeutic treatments compared to non-organized HA RBS inhibitors.

In various embodiments, each inhibitor is between about 41 A° and about 59 A°, about 42 A° and about 58 A°, about 43 A° and about 57 A° , about 44 A° and about 56 A°, about 45 A° and about 55 A°, about 46 A° and about 54 A° , about 47 A° and about 53 A° , about 48 A° and about 52 A° , about 49 A° and about 51 A°, and about 49.5 A° in distance from each other. In various embodiments, each inhibitor is at an angle of between about 58 degrees and about 62 degrees from each other, about 59 degrees and about 61 degrees from each other, or about 60 degrees from each other.

The scaffold may be of any molecule class, including but not limited to nucleic acids, polypeptides, organic molecules, inorganic molecules, lipids, carbohydrates, synthetic polymers, and combinations thereof. In one embodiment, the scaffold comprises a polypeptide scaffold, exemplified by those described herein. The polypeptide scaffold described herein comprise self-assembling polypeptides that, when assembled into a trimeric unit, can bind and organize the HA RBS inhibitors to possess the characteristics recited above (i.e.: that each inhibitor is between about 40 A° and about 60 A° in distance from each other and at an angle of between about 57 degrees and about 63 degrees from each other).

In one embodiment, the polypeptide scaffold comprises a trimeric polypeptide, wherein a monomeric unit of the trimeric polypeptide comprises a polypeptide at least 80% identical to a pub/peptide selected from the group consisting of SEQ ID NO:200-225. In various embodiments, a monomeric unit of the trimeric polypeptide comprises a polypeptide at least 80%, 85%, 90%, 91%, 92%, 93%, 94%, 95%, 96%, 97%, 98%, 99%, or 100% identical to a polypeptide selected from the group consisting of SEQ ID NO:200-225. As will be understood by those of skill in the art. in light of the disclosure herein, the monomeric units may comprise additional amino acid residues, including but not limited to other linkers as described herein.

The HA RBS inhibitors may be of any molecule class, including but not limited to nucleic acids, polypeptides, organic molecules, inorganic molecules, lipids, carbohydrates, synthetic polymers, and combinations thereof. In one embodiment, the HA RBS inhibitors comprises polypeptide RBS inhibitors, exemplified by those described herein, and exemplified by polypeptides including but not limited to SEQ ID NOS:1-82, 85-89, 95-97. 125-128, and 131-134. In one embodiment, the three HA RHS inhibitors are identical polypeptides.

In another aspect, the present invention provides methods for treating and/or limiting an influenza infection, comprising administering to a subject in need thereof a therapeutically effective amount of one or more polypeptides that includes the RBS inhibitor (such as a polypeptide RBS inhibitor), or an assembly of any embodiment of the invention, salts thereof, conjugates thereof, or pharmaceutical compositions thereof, to treat and/or limit the influenza infection. When the method comprises treating an influenza infection, the one or more polypeptides or assemblies are administered to a subject that has already been infected with the influenza virus, and/or who is suffering from symptoms (including but not limited to chills, fever, sore throat, muscle pains, coughing, weakness, fatigue, and general discomfort) indicating that the subject is likely to have been infected with the influenza virus. As used herein, “treat” or “treating” means accomplishing one or more of the following: (a) reducing influenza viral titer in the subject; (b) limiting any increase of influenza viral titer in the subject; (c) reducing the severity of flu symptoms; (d) limiting or preventing development of flu symptoms after infection; (e) inhibiting worsening of flu symptoms; (f) limiting or preventing recurrence of flu symptoms in subjects that were previously symptomatic for influenza infection.

When the method comprises limiting an influenza infection, the one or more polypeptides or assemblies are administered prophylactically to a subject that is not known to have been infected, but may be at risk of exposure to the influenza virus. As used herein, “limiting” means to limit influenza infection in subjects at risk of influenza infection. Given the nature of seasonal influenza outbreaks, virtually all subjects are at risk of exposure, at least at certain times of the year.

Groups at particularly high risk include children under age 18, adults over the age of 65, and individuals suffering from one or more of asthma, diabetes, heart disease, or any type of immunodeficiency.

As used herein, a “therapeutically effective amount” refers to an amount of the polypeptide or assembly that is effective for treating and/or limiting influenza infection. The polypeptides or assemblies are typically formulated as a pharmaceutical composition, such as those disclosed above, and can be administered via any suitable route, including orally, intranasally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Dosage regimens can be adjusted to provide the optimum desired response (e.g., a therapeutic or prophylactic response). A suitable dosage range may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. The polypeptides or assemblies can be delivered in a single bolus, or may be administered more than once (e.g., 2, 3, 4, 5, or more times) as determined by an attending physician.

In certain embodiments, the polypeptides or assemblies of the invention neutralize influenza virus infectivity. While not being limited by any mechanism of action, neutralizing activity may be achieved by preventing the influenza virus from interacting with its target cell. The polypeptides including the RBS inhibitor and assemblies of the invention target an HA epitope that blocks the receptor binding site of HA. Since the HA protein conformational change leads to fusion of the viral and cell membrane, polypeptide binding to the HA protein in its pre-fusion form may prevent fusion. In various embodiments, the polypeptides or assemblies of the invention prevent influenza virus from infecting host cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%. at least 20%, or at least 10% relative to infection of host cells by influenza virus in the absence of the polypeptides. Neutralization can, for instance, be measured as described in “Laboratory techniques in influenza,” edited by F. -X. Meslin, M. M. Kaplan and H. Koprowski (1996), 4th edition, Chapters 15-17, World Health Organization, Geneva.

The polypeptides or assemblies according to the invention can bind to the HA protein with any suitable affinity constant (K_{d} value) that provides therapeutic or prophylactic benefit. In various embodiments, the K_{d} value is lower than 0.2*10⁻⁴M, 1.0*10⁻⁵M, 1.0*10⁻⁶M, 1.0*10⁻⁷M, 1.0*10⁻⁸M, 1.0*10⁻⁹M, 1.0*10⁻¹⁰M, 1.0*10⁻¹¹M, or 1.0*10⁻¹²M. Affinity constants can for instance be measured using surface plasmon resonance, i.e., an optical phenomenon that allows for the analysis of real-lime biospecific interactions by detection of alterations in protein concentrations within a biosensor matrix, for example, using the BIACORE system (Pharmacia Biosensor AB, Uppsala, Sweden).

In another aspect, the present invention provides methods for diagnosing an influenza infection, or monitoring progression of an influenza infection, comprising

(a) contacting a biological sample from a subject suspected of having an influenza infection with a diagnostically effective amount of one or more polypeptides of the invention including the RBS inhibitor (such as a polypeptide RBS inhibitor) or assembly under conditions suitable for binding of the polypeptide or assembly to a viral HA protein present in the sample;

(b) removing unbound polypeptide or assembly, and/or sample; and

(c) detecting polypeptide-viral HA binding complexes or assembly-viral HA binding complexes,

where the presence of such binding complexes indicates that the subject has an influenza infection, or provides a measure of progression of an influenza infection.

The methods of this aspect of the invention can be used to more accurately identify patients that may be suffering from an influenza infection and to thus provide more informed determination of treatment options by an attending caregiver, individuals at risk of an influenza infection are as described above. The methods can also be used to monitor progression of an influenza infection; in this embodiment, the subject is known to be infected, and the methods can be used, for example, as a data point for an attending caregiver to determine whether to initiate, modify, or continue a particular course of therapy, such as treatment with neuraminidase or M2 protein inhibitors.

The biological sample may be any suitable biological sample including, but not limited to blood, serum, nasal secretions, tissue or other biological material from a subject at risk of infection.

The sample may first be manipulated to make it more suitable for the method of detection. “Manipulation” includes, but is not limited to treating the sample in such a way that any influenza virus in the sample will disintegrate into antigenic components such as proteins, polypeptides or other antigenic fragments. The polypeptides of the invention are contacted with the sample under conditions which allow the formation of a complex between the human polypeptides and influenza virus or antigenic components thereof that may be present in the sample. The formation of such complexes, if any, indicating the presence of influenza virus in the sample, is then detected and measured by suitable means. Such methods include, but are not limited to homogeneous and heterogeneous binding immunoassays, such as radioimmunoassays (RIA), ELISA, immunofluorescence, immunohistochemistry, FACS, BIACORE, biolayer interferometry and Western blot analyses. Suitable conditions to promote binding of the test compounds to one or more polypeptide of the invention can be determined by those of skill in the art, based on the teachings herein.

The polypeptides or assemblies of the invention for use in this aspect may comprise a conjugate as disclosed above, to provide a tag useful for any detection technique suitable for a given assay. The tag used will depend on the specific detection/analysis/diagnosis techniques and/or methods used. The methods may be carried in solution, or the polypeptide(s) of the invention may be bound or attached to a carrier or substrate, e.g., microtiter plates (ex: for ELISA), membranes and beads, etc. Carriers or substrates may be made of glass, plastic (e.g., polystyrene), polysaccharides, nylon, nitrocellulose, or teflon, etc. The surface of such supports may be solid or porous and of any convenient shape.

In another aspect, the invention provides methods for identifying candidate compounds for treating, limiting, and/or diagnosing influenza infection, comprising

(a) contacting an influenza HA protein with (i) test compounds and (ii) the polypeptide of any embodiment of the invention that includes the RBS inhibitor. the pharmaceutical compositions of the invention, or the assembly of any embodiment of the invention, or salts thereof, under conditions suitable for binding of the HA protein to the polypeptide of the present invention; and

(b) identifying those test compounds that outcompete the polypeptide for binding to the HA protein, wherein such test compounds are candidate compounds for treating, limiting, and/or diagnosing influenza infection.

In this aspect, the methods identify test compounds that compete with the polypeptides of the invention for binding to HA, and thus such candidate compounds may be useful in any of the other methods of the invention disclosed herein. Any suitable test compound can be used, as disclosed above in the aspect of the invention.

In general, competitive inhibition is measured by means of an assay, wherein an HA composition is admixed with the polypeptide(s) of the invention and the test compounds to be screened. In one embodiment, the test compounds to be screened are present in excess. Protocols based upon ELISAs are suitable for use in such competition studies. In certain embodiments, one may pre-mix the polypeptide(s) of the invention with varying amounts of test compounds to be screened (e.g., 1:10, 1:20, 1:30, 1:40, 1:50, 1 60, 1:70, 1:80, 1:90 or 1:100) for a period of time prior to applying to the HA composition. In other embodiments, the polypeptide(s) of the invention and varying amounts of test compounds to be screened are admixed during exposure to the HA composition. Any suitable detection means can be used binding. In one embodiment, the polypeptide(s) of the invention are tagged for detection, as discussed above. In this embodiment, the detectable label will decrease in the presence of competitive test compounds. The reactivity of the (labeled) polypeptide of the invention in the absence of test compound could serve as one suitable control. Preferably, competitive test compounds will, when present in excess, inhibit specific binding of the polypeptide(s) of the invention to HA by at least 10%, preferably by at least 25%, more preferably by at least 50%, and most preferably by at least 75% to 90% or even greater.

When the test compounds comprise polypeptide sequences, such polypeptides may be chemically synthesized or recombinantly expressed. Recombinant expression can be accomplished using standard methods in the art, as disclosed above. Such expression vectors can comprise bacterial or viral expression vectors, and such host cells can be prokaryotic or eukatyotic. Synthetic polypeptides, prepared using the well-known techniques of solid phase, liquid phase, or peptide condensation techniques, or any combination thereof, can include natural and unnatural amino acids. Amino acids used for peptide synthesis may be standard Boc (Na-amino protected Na-t-butyloxycarbonyl) amino acid resin with standard deprotecting, neutralization, coupling and wash protocols, or standard base-labile Na-amino protected 9-fluorenylmethoxycarbonyl (Emoc) amino acids. Both Fmoc and Boc Na-amino protected amino acids can be obtained from Sigma, Cambridge Research Biochemical, or other chemical companies familiar to those skilled in the art. In addition, the polypeptides can be synthesized with other Na-protecting groups that are familiar to those skilled in this art. Solid phase peptide synthesis may be accomplished by techniques familiar to those in the art and provided, such as by using automated synthesizers.

When the test compounds comprise antibodies, such antibodies can be polyclonal or monoclonal. The antibodies can be humanized, fully human, or murine forms of the antibodies. Such antibodies can be made by well-known methods. Such as described in Harlow and Lane, Antibodies; A Laboratory Manual, Cold Spring Harbor Laboratory, Cold Spring Harbor, N.Y., (1988).

When the test compounds comprise nucleic acid sequences, such nucleic acids may be produced by any suitable means, such as chemical synthesis. The nucleic acids may be DNA or RNA, and may be single stranded or double. Similarly, such nucleic acids can be chemically or enzymatically synthesized by manual or automated reactions, using standard techniques in the art. If synthesized chemically or by in vitro enzymatic synthesis, the nucleic acid may be purified prior to introduction into the cell. For example, the nucleic acids can be purified from a mixture by extraction with a solvent or resin, precipitation, electrophoresis, chromatography, or a combination thereof. Alternatively, the nucleic acids may be used with no or a minimum of purification to avoid losses due to sample processing.

When the test compounds comprise compounds other than polypeptides, antibodies, or nucleic acids, such compounds can be made by any of the variety of methods in the art for conducting organic chemical synthesis.

All of these aspects/embodiments disclosed herein can be combined with any other aspect/embodiment, unless the context clearly dictates otherwise,

### EXAMPLE 1

Computational Design of New Sialic-Acid Site Binders

A pre-selected list of 1718 high resolution monomeric crystal structures from the PDB, was searched for proteins that could harvest any fragments between Ser97 and Leu100K(Ala1001) of the heavy chain CDR3 of the c05 antibody(pdb 4fp8), resulting in grafted fragments of 6-15 residues. Variations of the loop were matched, allowing both endpoint matching as well as superposition. The shortest fragment contained only the tip of the hairpin and the few residues of the beta-strand-like conformation that hydrogen bond to Loop 130 of hemagglutinin (HA), which can be transplanted onto various beta-hairpin containing proteins. After integration of the matched segments, several rounds of ROSETTADESIGN™ along with rigid-body minimization were performed while ensuring that the hydrogen bonds to Loop 130 and Tyr98 were maintained. The new interface between the scaffold protein and HA was re-designed using ROSETTADESIGN™ while only keeping identities of the core beta turn region of the original loop fixed (amino acids: SAGW). Mutations in the designs were reverted to their original identity (the wildtype scaffold before design), if the reversion would not clash with the newly designed interface or would not harm the computed binding energy.

SB24 was based on the scaffold with the PDB 1.D. 2 qtd with the following sequence:

(SEQ ID NO: 130)

MINMKVAISMDVDKISNSFEDCKYFLIVRIDDNEVKSTKVIFNDESGKKS
IVKENVNAIICKNISEENYKKFSKKIEIYHAEGDDVDKNISLFIEGELSK
ISNP.

SB52 was based on the PDB entry 2w9q

(SEQ ID NO: 129)

GIVNVPNPNNTKFQELARFAIQDYNKKQNAHLEFVENLNVKEQVVAGIMY
YITLAATDDAGKKKIYKAKIWVKEWEDFKKVVEFKLV.

The sequence for SB24 was shortened in the progress of grafting, whereas no length changes were made to the SB52.

Design and Selection of Disulfide-Linked Circular Peptides against the Sialic Acid Site

To see whether a small disulfide-linked hairpin-like peptide would be able to mimic the sialic acid linked to galactose by α2,6-linkages (Siaα2,6Gal), which is displayed on the surface cells of the human respiratory tracts, we took the amino acids around the beta-turn of the CDR3 of the c05 antibody as a starting point. We replaced various positions to ensure stability of the small peptide as well as extra contacts. To ensure stability and folding of the peptide, we inserted two cysteine residues to allow the introduction of a disulfide bridge between the between the beta-strands of our model. The structure of the short peptide and its new disulfide bridge was designed using RosettaRemodel. Several variants were collected from the first library using yeast surface display.

### Results

### Designs Against the Sialic-Acid Binding Site

Designs were synthesized in bulk (Gen9 Inc.), and transformed as a pool together with a yeast surface display expression vector in to yeast cells. The pool of designs displayed on the surface of yeast was incubated with 1 μM H3 (A/Hong Kong/1/1968—H3N2) and yeast cells showing binding were selected. Indeed, several designs showed binding activity, however only if the graft contained the shortest fragment. Nine designs were identified that exhibited binding activity, three of these were sequence variations of the same scaffold and one bound non-specifically to other test proteins.

Sequences of experimentally identified computationally designed binders against HA are as follows:

SB24

(SEQ ID NO: 83)

GIGMVAISMDTDKISNSFEDCKYFLIVVVSAGWTNTIFNDESGKKEIVK
MNVDAIICKNISEENYKKFSKKIEIYHAEGDDVDKNISLFIEGELSKIS
NP;
SB52

(SEQ ID NO: 84)

GIVNVPNPNNTKFQQLARNAIANYNDNQNAHLEFVENLNVKEQVTGGIMY
YITLAATDDAGKKKIYKAKIAVVDSAGWEGVTEFKLV;
SB53

(SEQ ID NO: 85)

GIVNVPNPNNTKFQQLARNAIQNYNDNQNAHLEFVENLNVKEQVTGGIMY
YITLAATDDAGKKKIYKAKIAVVDSAGWEGITEFKLV;
SB55

(SEQ ID NO: 86)

GIVNVPNPNNTKFQQLARSAIQNYNDNQNAHLEFVENLNVKEQVTGGIMY
YITLAATDDAGKKKIYKAKIAVVDSAGWEGITEFKLV;
SB41

(SEQ ID NO: 87)

KEITNALETWGALGQDINLDIPSFQMSDDIADIKWEKTQDNKMIVVFSAG
WIAKDTYDLYENGTLKIAHLTTDDQAIYKVSITDTKGKNVLEKIFDLKIQ
ERVSKPKISWTCINTTLTCEVMNGTDPELNLYQDGKHLKLSQRVITHKWT
TSLSAKFKCTAGNKVSKESSVEPVSCPEK
SB60

(SEQ ID NO: 88)

KIIITGEPGVGKTTLVKKIVERLGKRAIGFWTEEVTDPETKKRTGFRIIT
TEGKKKVFSVVSAGWESKQNFEELAIPILERAYREAKKDRRKVIIIDEIG
DALGSSKFRDLVRQIHDPNVNVVATIPIRDDAPLIKEIRRLPGAVLIELT
PENRDVILEDILSLLER;
and
SB23

(SEQ ID NO: 89)

GHPTLKTPESVTGTWKGDVKIQCIYDPLRGYEQTEVKWLVRHGSDSVTIF
ERVSSAGWDGISQDKYNGRLQVSDSVPGDVSLQINTLQMDDRNHYTCEVT
WQTPDGNQVIRDKIIELRVRK.

### Sequence Modification of Two Selected Designs

Two designs (SB24 and S1352) were selected for further modification. Their genes were subjected to random mutagenesis and better binding protein variants were selected through yeast surface display selection against H3 (A/Hong Kong/1/1968—H3N2) and H2 (A/Adachi/2/1957—H2N2). The epitope was confirmed through competition with the S139/1 antibody for binding to H3; S139/1 binds to the sialic acid binding site as crystallographic analysis have confirmed. The fact that the designs could not bind when the S139/1 antibody was bound to heniaggiutinin strongly suggests that the designs indeed bind to the site they were designed for. Preliminary electron microscopy confirmed this data as well.

Improved Binding through Random Mutagenesis and Selections

A) SB24: individual mutations in the SB24 amino acid sequence identified after 3 rounds of selections against HA subtype H3 were 12T, V2 M, V281. or V28N4, V29E, and K49E.

Exemplary full length mutants are as follows:

H3 24-3 containing following mutations:

Variant H3 24-2 (V27M, F38S, K49E)

(SEQ ID NO: 90)

GIGMVAISMDTDKISNSFEDCKYFLIMVVSAGWTNTISNDESGKKEIVEM
NVDAIICKNISEENYKKFSKKIEIYHAEGDDVDKNISLFIEGELSKISNP
Variant H3 24-3a (I2T, V29E)

(SEQ ID NO: 91)

GTGMVAISMDTDKISNSFEDCKYFLIVVESAGWTNTIFNDESGKKEIVKM
NVDAIICKNISEENYKKFSKKIEIYHAEGDDVDKNISLFIEGELSKISNP
Variant H3 24-3b (V28M)

(SEQ ID NO: 92)

GTGMVAISMDTDKISNSFEDCKYFLIVMVSAGWTNTIFNDESGKKEIVKM
NVDAIICKNISEENYKKFSKKIEIYHAEGDDVDKNISLFIEGELSKISNP
LE
Variant H3 24-8 (V27M, K49E)

(SEQ ID NO: 93)

GTGMVAISMDTDKISNSFEDCKYFLIMVVSAGWTNTIFNDESGKKEIVEM
NVDAIICKNISEENYKKFSKKIEIYHAEGDDVDKNISLFIEGELSKISNP
LE

The binding behavior of these mutants was determined using yeast surface titration. It has been demonstrated before that binding to the head region, in particular to the sialic acid site, is highly effective for the neutralization of the virus with an effective concentration for neutralization (ECSO) close to the binding constant. Therefore, the SB24 variants provide great material for a potential therapeutics against Influenza viruses against various Influenza strains, as binding is relatively strong.

As the originally computational designs were designed to bind to H3, it was not surprising that they did not bind to the H1 nor H2 subtypes by using yeast surface display. To identify positions that were suboptimal, as well as positions that would allow binding to other subtypes, we generated a simple mutagenesis library of SB24. Screening of the library for binding to H3 and H2 allowed the identification of substitutions for SB24 to bind tighter to H3. No substitutions were found to enable binding of SB24 to H2, nor binding to H1 by either design library. We reasoned that binding to the other subtypes could require more than one or two substitutions.

Through next-generation sequencing, we obtained sequences for the naïve population as well as the population after selection 1 and 2. for binding to H3 and mapped out optimality of each position.

The results of these studies confirmed that the interface between the SB24 mutant polypeptides and the HA protein are around residues 1-3, 25-38, 45-51, and position 83. Heat maps (not shown) were generated showing how optimal or variable an amino acid at a given position at the interface residues of the SB24 mutants is in the context of binding against subtype H3 by determining the frequency of an amino acid change in naïve as well as sorted pool, followed by calculating the ratio of selected versus naïve frequencies of a given amino acid change of a given position.

Positions that had an effect on binding above average based on the first modification step and extracted from the heat map were interface positions 1, 2, 26-29, 38, 45, 49, 51. We additionally identified some small effect on binding through the positions 71-73 and 90-93, which are part of the variability regions, and changes within these positions likely contribute indirectly to binding, e.g. by stabilizing the protein. Changes and positions identified were included into the next generation sequence diversity discussed below and summarized together with additional changes for the next generation of improved binders in Table 4.

B) SB52: Individual Beneficial Mutations in the SB52 Amino Acid Sequence identified after 3 rounds of selections against HA subtype H3 were N195 or N19Y, L38M, and G47D.

Exemplary full length mutants were as follows:

Variant SB52-3 (G47D, N27Y)

(SEQ ID NO: 94)

GIVNVPNPNNTKFQQLARNAIANYNDYQNAHLEFVENLNVKEQVTGDIMY
YITLAATDDAGKKKIYKAKIAVVDSAGWEGVTEFKLV
52-N19S

(SEQ ID NO: 121)

GIVNVPNPNNTKFQQLARSAIANYNDYQNAHLEFVENLNVKEQVTGDIMY
YITLAATDDAGKKKIYKAKIAVVDSAGWEGVTEFKLV
52-N19Y

(SEQ ID NO: 122)

GIVNVPNPNNTKFQQLARYAIANYNDYQNAHLEFVENLNVKEQVTGDIMY
YITLAATDDAGKKKIYKAKIAVVDSAGWEGVTEFKLV
52-L38M

(SEQ ID NO: 123)

GIVNVPNPNNTKFQQLARNAIANYNDYQNAHLEFVENMNVKEQVTGDIMY
YITLAATDDAGKKKIYKAKIAVVDSAGWEGVTEFKLV
52-G47D

(SEQ ID NO: 124)

GIVNVPNPNNTKFQQLARNAIANYNDYQNAHLEFVENLNVKEQVTDDIMY
YITLAATDDAGKKKIYKAKIAVVDSAGWEGVTEFKLV
Variant SB52-3 (G47D, N27Y)

(SEQ ID NO: 94)

GIVNVPNPNNTKFQQLARNAIANYNDYQNAHLEFVENLNVKEQVTGDIMY
YITLAATDDAGKKKIYKAKIAVVDSAGWEGVTEFKLV

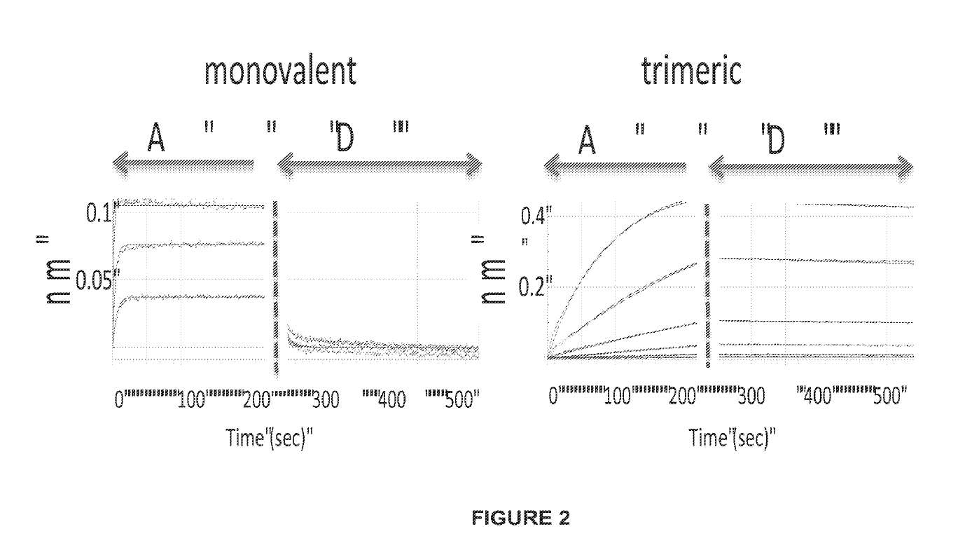

Binding behavior of exemplary SB52 mutants, as determined using yeast surface titration, is provided in FIG. 2. For example, variant 52NC-2 (SEQ ID NO:96) shows binding to all three subtypes H1, H2 and H3, which is ideal for the making of a potential new therapeutic.

Variants of 5B52 for Binding to H2

Influenza A can be phylogenetically described as two major groups. As the previous designs were made against H3, binders against the H2 subtypes are desirable. We were able to identify SB52 mutations that allow cross-specific binding between the group I subtype H2 and the group II subtype H3. Identified beneficial mutations were N7K, N19Y, E79V, and I21V based on a simple random mutagenesis library. Exemplary full length mutants were as follows:

Variant H2 SB52-4 (N19Y, E79V)

(SEQ ID NO: 13)

GIVNVPN(P/C)NNTKFQQLARYAIANYNDNQNAHLEFVENLN(V/C)KE
QVTGGIMYYITLAATDDAGKKKIYKAKIAVVDSAGWVGVTEFKLV
Variant H2 SB52-13 (N7K, N19Y, E79V)

(SEQ ID NO: 14)

GIVNVPK(P/C)NNTKFQQLARYAIANYNDNQNAHLEFVENLN(V/C)KE
QVTGGIMYYITLAATDDAGKKKIYKAKIAVVDSAGWVGVTEFKLV

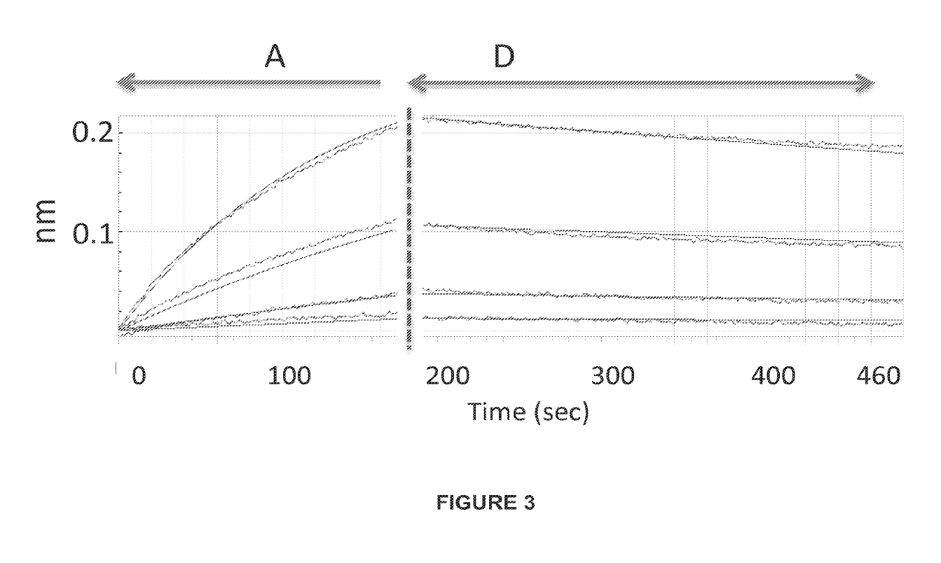

Binding behavior of exemplary SB52 mutants, as determined using yeast surface titration, is provided in FIG. 3. Different binding signals for diverse variants and hemagglutinin versions demonstrates the variants potential to serve as diagnostics, as it can discriminate strains or subtypes. This can be tremendously useful in clinical settings to identify which Influenza strain or subtype has infected a patient. Having a diverse set of variants with different specificity could allow providing a panel of these variants on a detection device to identify clearly which strain is dealt with.

As the originally computational designs were designed to bind to H3, it was not surprising that they did not bind to the H1 nor H2 subtypes by using yeast surface display. To identify positions that were suboptimal, as well as positions that would allow binding to other subtypes, we generated a simple mutagenesis library of SB52. Screening of the library for binding to H3 and H2 allowed the identification of substitutions for SB52 to bind tighter to H3, as well as substitutions in SB52 that allow binding to H2. No substitutions were found to enable binding of to H1. We reasoned that binding to the other subtypes could require more than one or two substitutions. For example, for S52, at least two substitutions were necessary to obtain binding to H2. Through next-generation sequencing, we obtained sequences for the naïve population as well as the population after selection 1 and 2 for binding to H3 and mapped out optimality of each position.

The results of these studies indicate that the interface between the SB52 mutant polypeptides and the HA protein are at around residues 18-27, 44-48, and 70-82.

### SB52 Against H3

Heal maps (not shown) were generated showing how optimal or variable an amino acid at a given position at the interface residues of the SB52 mutants is in the context of binding against subtype H3 by determining the frequency of an amino acid change in naïve as well as sorted pool, followed by calculating the ratio of selected versus naïve frequencies of a given amino acid change of a given position.

Positions that had an effect on binding above average based on the first modification step and extracted from the heat map were interface positions: 19, 23, 26, 27, 45-48, 71, 74, 79, 80 and 82. We additionally identified some small effect on binding through the positions 11, 13, 51, 59, 64, 67-69 and 86 which are part of the variability regions, and changes within these positions likely contribute indirectly to binding, e.g, by stabilizing the protein.

Modification of Binding through Combinatorial Libraries and Selections

Hemagglutinin is subject to constant genetic changes. This is particularly manifested in the head region of hernaggilutinin, as most antibodies bind to this exposed area: whenever a mutation of HA occurs, which prevents binding of any existing antibodies in an infected individual, the virus can propagate efficiently. Hence, the head region is under constant selective pressure to evade the immune response, so changes within the head region area tend to improve the survival of the virus and a new seasonal virus can evolve. For designing protein binders and inhibitors against the head region of HA that partially overlap with eas that are subject to change, it is important to provide enough diversity to the inhibitor library to accommodate those changes. The core part of the binder/inhibitor (S852 and SB24) binds to the sialic acid binding site which cannot mutate, otherwise the virus would not be able to infect cells. However, changes at the periphery may occur and the aim of the generation of further libraries was to introduce diversity within the sequence of the binders that would accommodate those changes. The constant genetic drift of HA introduces charge inversions, protrusions, insertions and deletions within the periphery of the sialic acid binding site of HA itself. Hence, for positions of SB24 that are close to areas of high sequence variations of HA, corresponding diversity is needed in the inhibitor to avoid charge repulsions or clashes. As we had a model of the interactions between SB24 and HA, the sequence variation became a spatially defined problem for which we could rationally decide on the diversity needed to compensate for the genetic drift of the influenza virus, at least for its surface protein HA. For instance, if HA would introduce a negatively charged residue, the binder cannot have a negatively charge residue right next to it as they would repel each other and weaken binding significantly. For the design of the generation II library, sequence changes of HA from different subtypes and strains were considered in the context of the three-dimensional model bound to the head region of HA. Thereby, corresponding sequence changes necessary to accommodate the diversity of HA were allowed. The overall idea is that if a single universal design against the head region is not possible, we will have a small library of variants front which the appropriate one for a given strain can be quickly pulled out of.

Next to mutations that would allow binding to other subtypes, several beneficial mutations that were identified through previous selection and deep sequencing were incorporated into the generation II libraty.

### (A) SB24 Variants

Variants below were isolated after selections against either H1 or H2 subtypes (exemplified by A/Hong Kong/1/1968, HIN1 A/Solomon Islands/3/2006 (HI SI), H2N2 A/Adachi/2/1957 (H2 Adachi) or A/New Caledonia/20/1999) or as a combination of these.

SB24solo2

(SEQ ID NO: 45)

ASGMLAISMDTDKISNSFEDCKYFLIELKSAGWTNTIFNDESGKEEIVKM
GVDAIICKNISEENYKKFSKKIEIYHAEGDDVNKNISLFLEGELSKISNP
SB24solo6

(SEQ ID NO: 46)

ASGMLAISMDTDKISNSFEDCKYFQLELPSAGWTNTIFNDESGKEEIVK
MNVDAIICKNISEENYKKFSKKIEIYHAEGDDVKKNISLFLEGELSKIS
NP;
SB24NC1

(SEQ ID NO: 47)

ASGMLAISMDTDKISNSFEDCKYFQIELPSAGGTNTIFNDESGKEEIVK
MGVDAIICKNISEENYKKFSKKIEIYHAEGDDVKKNISLFIEGELSKIS
NP;
SB24alt1-1

(SEQ ID NO: 125)

ASGMLAISMDTDKISNSFEDCKYFQLYVLSAGWTNTIFNDESGKEEIVK
MSVDAIICKNISEENYKKFSKKIEIYHAEGDDVDKNISLFLEGELSKIS
NP;
SB24SingNC1

(SEQ ID NO: 127)

ASGMLAISMDTDKISNSFEDCKYFQIELPSAGWTNTIFNDESGKEEIVK
MGVDAIICKNISEENYKKFSKKIEIYHAEGDDVEKNISLFLEGELSKIS
NP;
and
SB24SingNC3

(SEQ ID NO: 128)

ASGMLAISMDTDKISNSFEDCKYFQLVLASAGWTNTIFNDESGKEEIVK
MGVDAIICKNISEENYKKFSKKIEIYHAEGDDVDKNISLFAEGELSKIS
NP.

(B) SB52 Variants

The following sequence diversity was used for the SB52 library generation, based on the previous studies:
Library Composition:
Position 10: T only.
Position 13: Y or F (wt)
Position 18: N, Y, T, S
Position 21: V or I
Position 26: N,H and Y
Position 27: N, H and Y
Position 44: L, T, V, A, S, I
Position 45: A, G, S, N, T, Y
Position 46 and 47 are set so that one of them is glycine and the other is any of the following 17 amino acids; N, H, D, S, K, Y, H, A, V, T, I, Q, D, E, M, L, F
Position 48: N, G, T, I, A, S, D, V
Position 70: V, I
Position 71: G, A
Position 74: E
Position 79: K,I, A, T, V, E
Position 80: V, A, G
Position 82: T, K, R, D, E, A, S, N

SB52 variants below were isolated after selections against H1, H2, or H3 subtypes (exemplified by either H3 A/Hong Kong1968, H3 A/Victoria/361/2011 (H3 Victoria), H1 A/Solomon Islands/3/2006 (H1 S1), H1 A/South Carolina/1/1918 (H1 1918), H1 A/California/04/2009 (Cal 09), H1N1 A/Singapore/6/1986 (H1 Singapore) H₁ A/New Caledonia/20/1999 or H2 A/Adachi/2/1957 (H2 Adachi):

52solo1

(SEQ ID NO: 15)

GIVNVPN(P/C)NTTKYQQLARSAIAIYNYHQNAHLEFVENLN(V/C)KE
QVGGMNMYYITLAATDDAGKKKIYKAKVGVVESAGWKGVEEFKLV;
52solo2

(SEQ ID NO: 16)

GIVNVPN(P/C)NTTKYQQLARSAIAVYNYHQNAHLEFVENLN(V/C)KE
QIAGKTMYYITLAATDDAGKKKIYKAKVGVVESAGWEGVEEFKLV;
52solo3

(SEQ ID NO: 17)

GIVNVPN(P/C)NTTKYQQLARIAIAVYNNYQNAHLEFVENLN(V/C)KE
QSGGKVMYYITLAATDDAGKKKIYKAKVGVVESAGWIGVEEFKLV;
52solo8

(SEQ ID NO: 18)

GIVNVPN(P/C)NTTKYQQLARSAIAVYNNHQNAHLEFVENLN(V/C)KE
QAGGKAMYYITLAATDDAGKKKIYKAKIGVVESAGWEGVEEFKLV;
52sing2

(SEQ ID NO: 19)

GIVNVPN(P/C)NTTKYQQLARRAIAIYNNNQNAHLEFVENLN(V/C)KE
QTGLGIMYYITLAATDDAGKKKIYKAKIAVVESAGWVAVSEFKLV;
52solo8a

(SEQ ID NO: 20)

GIVNVPN(P/C)NTTKYQQLARSAIAVYNNHQNAHLEFVENLN(V/C)KE
QAGGKAMYYITLAATDDAGKKKIYKAKIGVVESAGWEGVEEFKLV;
52solo8b

(SEQ ID NO: 21)

GIVNVPN(P/C)NTTKYQQLARSAIAVYNYHQNAHLEFVENLN(V/C)KE
QIAGKTMYYITLAATDDAGKKKIYKAKVGVVESAGWEGVEEFKLV;
52soloc

(SEQ ID NO: 22)

GIVNVPN(P/C)NTTKYQQLARSAIAVYNNHQNAHLEFVENLN(V/C)KE
QAGGKAMYYITLAATDDAGKKKIYKAKIGVVESAGWEGVEEFKLV;
52sing2

(SEQ ID NO: 23)

GIVNVPN(P/C)NTTKYQQLARRAIAIYNNNQNAHLEFVENLN(V/C)KE
QTGLGIMYYITLAATDDAGKKKIYKAKIAVVESAGWVAVSEFKLV;
52sing2W

(SEQ ID NO: 24)

GIVNVPN(P/C)NTTKYQQLARRAIAIYNNNQNAHLEFVENLN(V/C)KE
QWGLGIMYYITLAATDDAGKKKIYKAKIAVVESAGWVAVSEFKLV;
52sing2D

(SEQ ID NO: 25)

GIVNVPN(P/C)NTTKYQQLARRAIAIYNNNQNAHLEFVENLN(V/C)KD
QTGLGIMYYITLAATDDAGKKKIYKAKIAVVESAGWVAVSEFKLV;
52alt1-3

(SEQ ID NO: 26)

GIVNVPN(P/C)NTTKYQQLARSAIAIYNNHQNAHLEFVENLN(V/C)KE
QVSYGAMYYITLAATDDAGKKKIYKAKVGVVESAGWVGVEEFKLV;
52alt1-4

(SEQ ID NO: 27)

GIVNVPN(P/C)NTTKYQQLARSAIAVYNYHQNAHLEFVENLN(V/C)KE
QIDYGAMYYITLAATDDAGKKKIYKAKIGVVESAGWIGVEEFKLV;
52del1

(SEQ ID NO: 31)

GIVNVPN(P/C)NTTKYQQLARRAIAVYNYYQNAHLEFVENLN(V/C)KE
QSSGLDMYYITLAATDDAGKKKIYKAKIAVESAGWIVVTEFKLV;
52del4

(SEQ ID NO: 32)

GIVNVPN(P/C)NTTKYQQLARRAIAVYNYYQNAHLEFVENLN(V/C)KE
QLTGHGMYYITLAATDDAGKKKIYKAKVAVESAGWIVVTEFKLV;
52del2

(SEQ ID NO: 33)

GIVNVPN(P/C)NTTKYQQLARRAIAVYNYYQNAHLEFVENLN(V/C)KE
QAGGQVMYYITLAATDDAGKKKIYKAKVAVESAGWIVVTEFKLV;
52del3

(SEQ ID NO: 34)

GIVNVPN(P/C)NTTKYQQLARRAIAVYNYYQNAHLEFVENLN(V/C)KE
QAAGEIMYYITLAATDDAGKKKIYKAKIAVESAGWIVVREFKLV;
52del5

(SEQ ID NO: 35)

GIVNVPN(P/C)NTTKYQQLARRAIAVYNYYQNAHLEFVENLN(V/C)KE
QAAGEIMYYITLAATDDAGKKKIYKAKIAVESAGWIVVREFKLV;
52del6

(SEQ ID NO: 36)

GIVNVPN(P/C)NTTKYQQLARIAIAIYNYHQNAHLEFVENLN(V/C)KE
QSGHGTMYYITLAATDDAGKKKIYKAKVGVVESAGWIGVTEFKLV;
52del7

(SEQ ID NO: 37)

GIVNVPN(P/C)NTTKYQQLARIAIAVYNHHQNAHLEFVENLN(V/C)KE
QISVGTMYYITLAATDDAGKKKIYKAKVGVVESAGWIEVEEFKLV;
52del8

(SEQ ID NO: 38)

GIVNVPN(P/C)NTTKYQQLARRAIAVYNYYQNAHLEFVENLN(V/C)KE
QLTLGVMYYITLAATDDAGKKKIYKAKVAV-ESAGWIVVTEFKLV;
52del9

(SEQ ID NO: 39)

GIVNVPN(P/C)NTTKYQQLARRAIAVYNYYQNAHLEFVENLN(V/C)KE
QSSSGNMYYITLAATDDAGKKKIYKAKIAV-ESAGWIVVKEFKLV;
52vic5-2

(SEQ ID NO: 28)

GIVNVPN(P/C)NTTKYQQLARRAIAIYNNNQNAHLEFVENLN(V/C)KE
QWGLGIMYYITLAATDDAGKKKIYKAKIAVVESAGWVAVSEFKLV;
52vic5-3

(SEQ ID NO: 29)

GIVNVPN(P/C)NTTKYQQLARSAIAIYNNNQNAHLEFVENLN(V/C)KE
QLNGYDMYYITLAATDDAGKKKIYKAKIGVVESAGWTGVNEFKLV;
52vic5-4

(SEQ ID NO: 30)

GIVNVPN(P/C)NTTKYQQLARSAIAIYNHYQNAHLEFVENLN(V/C)KE
QITGYDMYYITLAATDDAGKKKIYKAKVAVVESAGWEVVAEFKLV;
52NC-1

(SEQ ID NO: 95)

GIVNVPN(P/C)NTTKYQQLARIAIAVYNHHQNAHLEFVENLN(V/C)KE
QLGEGDMYYITLAATDDAGKKKIYKAKVGVVESAGWTGVEEFKLV;
52NC-2

(SEQ ID NO: 96)

GIVNVPN(P/C)NTTKYQQLARSAIAIYNYHQNAHLEFVENLN(V/C)KE
QLGEGDMYYITLAATDDAGKKKIYKAKIGVVESAGWTGVEEFKLV;
52NC-8

(SEQ ID NO: 97)

GIVNVPN(P/C)NTTKYQQLARSAIAIYNYHQNAHLEFVENLN(V/C)KE
QIGEGSMYYITLAATDDAGKKKIYKAKVGVVESAGWEGVEEFKLV;
and
52solo6

(SEQ ID NO: 126)

GIVNVPN(P/C)NTTKYQQLARRAIAIYNHNQNAHLEFVENLN(V/C)KE
QIDYGSMYYITLAATDDAGKKKIYKAKVGVVESSGWTGVEEFKLV.

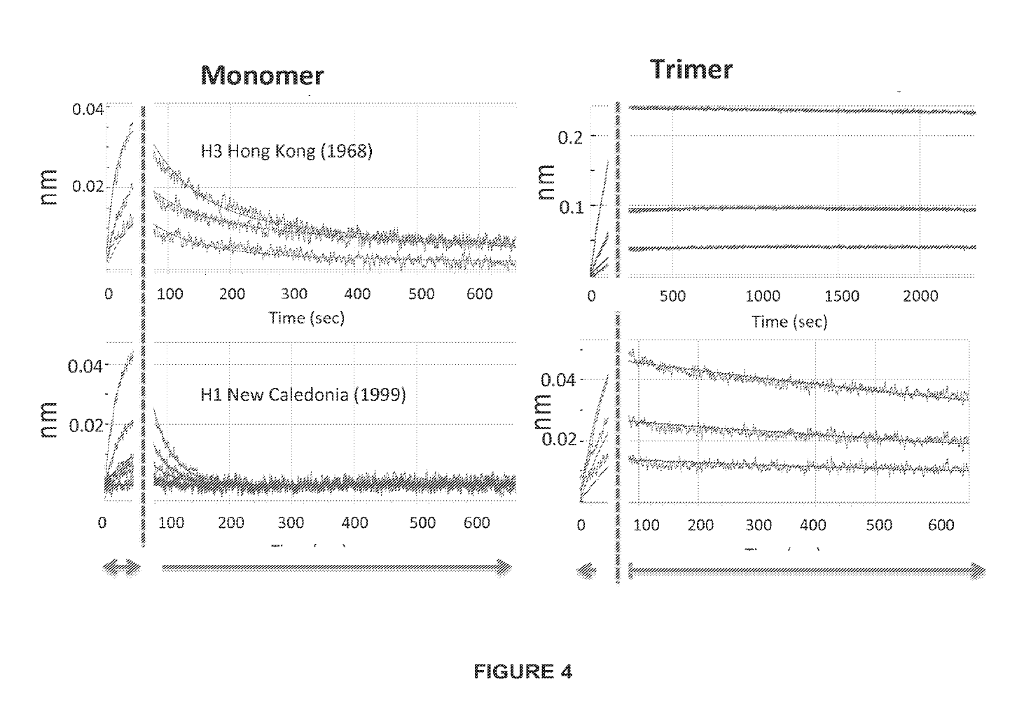

Table 11 shows binding data for exemplary mutants using yeast surface display, and demonstrate that several variants were identified that can either have strong binding to H1and H3, or H5 subtypes or even all three H1, H2 and H3 as 52NC,-2 (SEQ ID NO:96). * refers to any 52del variants listed in the document. As blocking the receptor binding site of Influenza also inhibits the virus, these variants present a new material for therapeutic avenues. As FIGS. 3 and 4 (barcharts) demonstrate, specific binders can also be quickly identified based on these polypeptide sequences, and we present several of these here. This high specificity will be tremendously useful for the manufacture of the next-generation flu diagnostics. The high specificity of the individual variants based on SB52 and SB52 can provide a set of variants that will allow paneling of the different Influenza strains.
Sequence Variations and Binding Data

TABLE 11
Determination of affinity constants via yeast surface display. Concentration
displayed in nM.

| | 52alt1-3 | 52solo2 | 52solo1 | 52Sing2 | 52NC-2 | 24alt1-1 | 24solo2 | 52del* |
|---|---|---|---|---|---|---|---|---|
| H1 (A/Solomonislands/3/2006) | − | 1.7 | 1.8 | 17.2 | 4.4 | | | nd |
| H1 (A/NewCaledonia/20/1999) | − | 9.1 | 20.7 | 44.9 | 27.2 | 436 | 23.2 | nd |
| H1 (A/Singapore/6/1986) | nd | nd | nd | ++ | nd | | | nd |
| H1 (A/South Carolina/1/1918) | − | + | + | + | | | | nd |
| H2 (A/Adachi/2/1957) | 8.5 | − | >350 | − | 9.6 | − | − | nd |
| H3 (A/Hong Kong/1/1968) | 3 | 1 | 35.66 | − | 3.9 | 3.7 | +++ | nd |
| H1 (California/2009) | (+) | − | − | + | − | − | − | nd |
| H5 A/Vietnam/1203/2004 | − | − | − | − | − | − | − | ++ |

### Generation of Disulfide-Linked Peptides

HA mediates the attachment to the host-cells, As this function is so central to viral propagation, its sequence can vary only to the extent that it does not disrupt the functions of HA. Thus, this functional site presents an Achilles' heel for targeted protein therapeutics. Despite the strong evolutionary constraints which maintain binding of HA to its substrate (sialic acid on the surface of the host cells), substantial sequence changes occur in the immediate proximity of this binding site, and only very few residues are conserved, providing few constant contacts to bind to. For this reason it is can be difficult to obtain a high affinity inhibitor without compromising the breadth of binding to various HA subtypes. After all, the antigenic drift is part of its protecting mechanism against the host adaptive immune response. Also binding of HA to its receptor sialic acid itself is very weak (low mM). Hence, the smaller a binding protein is, the higher the chance that it would not interfere with any residues that are subject to constant changes and would thereby escape by blocking binding with the introduction of an incompatible residue at the interface.

For the rational design of peptides binding to the sialic acid site of hemagglutinin, the polypeptide can assume a hairpin-like structure that should be stabilized through a disulfide bridge at the hairpin ends. Thus we introduced pairing cysteine residues at a non-hydrogen bonding beta strand pair, which were positions at positions 3 and 14. Peptides contain HM (N-terminal) and LE (C-terminal) as part of the cloning sites and was tested a fusion C-terminal of the Aga2 protein (yeast surface anchor) and N-terminal of the c-Myc tag.

The library was screened for the best variants. Exemplary peptides include (where “X: is any amino acid):

(SEQ ID NO: 48)

GXCIGVPSAGWEXCW;

(SEQ ID NO: 49)

GGCIGVPSAGWEWCP;

(SEQ ID NO: 50)

GGCLGVPSAGWEICW;

(SEQ ID NO: 51)

GWCIGVPSAGWEICW;

(SEQ ID NO: 52)

GRCIGVPSAGWEVCW;

(SEQ ID NO: 53)

GHCMGVASAGWEICW;

(SEQ ID NO: 54)

GDCIGVASAGWEWCP;

(SEQ ID NO: 55)

GGCIGVPSAGWEWCP;

(SEQ ID NO: 56)

GGCLGVPSAGWEWCP;

(SEQ ID NO: 57)

GWCIGVPSAGWEICW;

(SEQ ID NO: 58)

GWCIGVPSAGWEICW;

(SEQ ID NO: 59)

GRCIGVPSAGWEVCW;

(SEQ ID NO: 60)

GHCMGVASAGWEICW;

(SEQ ID NO: 61)

GDCIGVASAGWEWCP;

(SEQ ID NO: 65)

GSCYRVVSAGWETC;

(SEQ ID NO: 65)

GSCYRVVSAGWETC;

(SEQ ID NO: 67)

GGCARVASAGWEICN;

(SEQ ID NO: 68)

GKCRWVASAGWEVCA;

(SEQ ID NO: 69)

GNCFAVVSAGWEKCK;

(SEQ ID NO: 70)

GMCTHVLSAGWEPCL;

(SEQ ID NO: 71)

GMCTHVLSAGWEPCL;

(SEQ ID NO: 72)

GCCGYVISAGWEMCS;

(SEQ ID NO: 73)

GDCTCMISAGWEPCE;

(SEQ ID NO: 74)

GFCCLVTSAGWEECY;

(SEQ ID NO: 75)

GFCCLVTSAGWEECY;

(SEQ ID NO: 76

GSCPFVTSAGWEKCL;
and

(SEQ ID NO: 66)

GDCIVVASAGWEACR

### EXAMPLE 2

### Self-Sssembling Trimeric Protein Units for Modification of Binding to the Influenza's Hemagglutinins Head-Region

### Summary

The non-limiting example described here concerns polypeptides that organize into trimeric units to display prior disclosed receptor binding site (RBS) inhibitors in alignment with the RBS of the trimeric hemagglutinin of influenza. The genetic fusion of the receptor binding site (RBS) inhibitors with the here disclosed self-assembling polypeptides allows the interaction of multiple units of the RBS inhibitor with the hemagglutinin, resulting in a significant boost of affinity. Thereby, the fusions described here provide enhanced performance to provide new reagents for diagnostics and therapeutic treatments. Preliminary analysis demonstrated that the trimeric fusions inhibit infectivity of the influenza virus.

The self-assembling protein complexes were identified by searching pre-existing trimeric units that would fit onto either termini of the model of three units of the previously disclosed binders bound to the trimeric hemagglutinin, and clashing residues were re-designed.

### Geometric Description

The Influenza virus has two major coat proteins, neuramidase (NA) and hemagglutinin (HA). HA commonly mediates both the attachment to the host-cells and the fusion between the viral and host membranes, leading to the release of the viral genome into the cytoplasm of the host cell. HA consists of three identical units assembled into its characteristic mushroom-shaped form. The head-shaped section contains the receptor binding site which targets sialic acid moieties on the surface of the host cells, whereas the stem region contains the functional site responsible for the fusion with the host membrane. As the functional unit is trimeric, three receptor binding sites are displayed on the surface of the head-region. Blocking the receptor binding site enables neutralization and inhibition of the virus. The relative location of the receptor binding site can be described as an equilateral triangle.

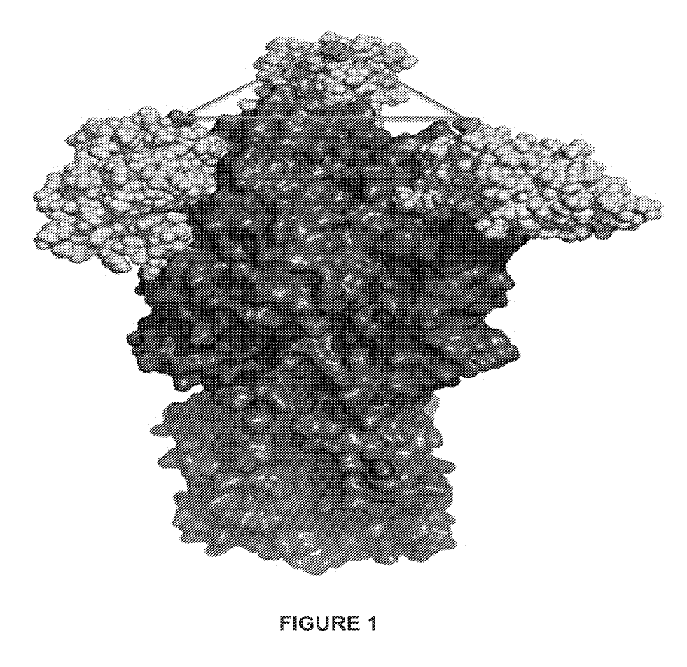

This example describes self-assembling trimeric protein units that allow ideal positioning of monomeric binding units/domains targeting the head region. FIG. 1 demonstrates the model of 3 units of the previously disclosed SB52 variants bound to trimeric hemagglutinin. Their N-terminus has an approximate distance of 49.5 A from each other, with each being approximately 60 degrees from each other. The disclosed proteins are genetic fusions to the termini to ensure the position of the receptor binding site proteins accordingly to the geometric arrangement.

### Computational Design of Trimeric Binding Units

For the search, we sampled translations and rotations of 600 homotrimeric structures along the symmetry axis of the HA trimers and scored proximity to either termini while avoiding clashes. Out of the successful solutions, we focused on small trimers of the thermophilic organisms with compatible geometry for loop closure between the identified nimeric adapter and SB52 variant. Residues of the trimer unit that could interfere with binding to HA were manually “shaved off” to avoid effects on the interaction between SB52 with HA, e.g. charges were designed to become neutral residues, and potentially clashing bulky residues were changed to smaller ones using FoldIt(1). The loops were typically closed with a Gly-Ser linker, which encodes the restriction site for BamHI. This setup was chosen to facilitate the versatility of the adapter to allow quick cloning of other variants into their trimeric form. For initial experimental characterization, we fused the trimer adapter to the SB52NC-2 (SEQ ID NO: 96) variant. Different loop lengths were tried out and expression and solubility were monitored.

Loops between the trimerization and binding domains were closed via ROSETTAREMODEL™ (2) Blueprint files were edited so that either 2, 3, 5 or 8 residues were modeled; the top scoring solution out of 50 was kept.

### Trimeric Self-Assembling Units

The following exemplary polypeptides self-assemble to form trimers. All HAA.x.0 sequences can additionally have a linker at the C-terminus, including but not limited to those described herein.

HAA.1 (used, for example, as N-terminal fusion to SB52 variants, though can be used with other RBS inhibitors) SEQ ID NO: 200 MEEVVLITVPSEEVARTIAKALVEERLAACVNIVPGLTSIYRWQGEVVED QELLLLVKTTTHAFPKLKERVKALHPYTVPEIVALPIAQGNQEYLDWLRENT

HAA.2 (used, for example, as N-terminal fusion to SB52 variants, though can be used with other RBS inhibitors) SEQ ID NO: 227 MEEVVLITVPSEEVARTIAKALVEERLAACVNIVPGLTSIYRWQGEVVED QELLLLVKTTTHAFPKLKERVKALHPYTVPEIVALPIAQGNQEYLDWLRENA

HAA.1.1 SEQ ID 201 (used, for example, as N-terminal fusion to 5B52 variants, though can be used with other RBS inhibitors) MEEVVLITVPSEEVARTIAKALVEERLAACVNIVPGLISIYRWQGEVVED QELLLLVKTTTAAFPKLKERVKALHPYTVPETVALPIAEGNREYLDWLQEN

HAA.2 (used, for example, as N-terminal fusion to SB52 variants, though can be used with other RBS inhibitors) SEQ ID 202 ALYFSGHMILVYSTFPNEEKALEIGRKLLEKRLIACFNAFEIRSGYWWKGEIV QDKEWAAIFKTfQSKQKELYEELRKLHPYETPAIFTLKVENLTEYMNWLAES VLGS

HAA.3 (used, for example, as N-terminal fusion to SB52 variants, though can be used with other RBS inhibitors) SEQ ID 203 MKLIVAIVRPEKINEVLKAIFQAEVRGLTLSRVQGHGMELHEKVRLEIGVSEP FVKPTVEAILKAARTGEVGDGICIFVLPVEKVYRIRTGEEA

HAA.3 (used, for example, as N-terminal fusion to SB52 variants, though can be used with other RBS inhibitors) SEQ ID 204 MKLIVAIVRPEKLNEVLKALFQAEVRGLTLSRVQGHGGETERVETYRGTTVK MELHEKVRLEIGVSEPFVKPTVEAILKAARTGEVCiDGKIFVLPVEKVYRIRTG EEA

HAA.4 (used, for example, as N-terminal fusion to SB24 variants, though can be used with other RBS inhibitors) SEQ ID 205 KMEELFKKHKIVAVLRANSVEEAKEKALAVFEGGVHLIEITFTVPDADTVIKE LSFLKEKGAIIGAG TVTSVEQCRKAVESGAEFINSPHIDEEISQFCKEKGVFYM PGVMTPTELVKAMKLGHTILKLFPGQVVGPQFVKAMKGPFPNVKFVPTGGV NLSNVAAWFAAGVLAVGVGSALVKGTPNNVREKAKAFVEAIRGA

HAA.5 (used, for example, as N-terminal fusion to SB52 variants, though can be used with other RBS inhibitors) SEQ ID 206 KDSEIVKALGDLDELNSVLGVVSSLYPSLSQVIQKLQNDIFSISSEIAGFDMNFS DEKVKGIEELITNYSKELEPLSNFVLPGGHIASSFLHLARAVCRRAERSVVTLL KESKAKEVHAKYLNRLSSLLFVLALVVNKRTSNPNVIWR

HAA.6 (used, for example, as N-terminal fusion to SB24, variants, though can be used with other RBS inhibitors) SEQ ID 207 SKDSPIIEANGTLDELTSFIGEAKHYVDAEMQGILEEIQNDIYKIMGEIGSKGKI EGISEERTKWLEGLISRYEEMVNLKSFVLPGGTLESAKLDVCRTIARRAERKV ATVLREFGIGKEALVYLNRLSDLLFLLARVIEIE

HAA.7 (used, for example, as N-terminal fusion to SB52 variants, though can be used with other RBS inhibitors) SEQ ID 208 GSMKKVEAIIRPEKLEIVKKALSDAGYVGMTVSEVKGRGVQGGIVERYRGRE YIVDLIPKVKIELVVKQADVDNVIDIICENARTGNPGDGKIFVIPVQRVVRVRT KEEGAAALLEH

HAB.1 (used, for example, as N-terminal fusion to SB52 variants, though can be used with other RBS inhibitors) SEQ ID 209 ILVYSTFPNEEKALEIGRKLLEKRLIACFNAFEIRSGYWWKGiEIVQDKEWAAIF KTTEEKEKELYEELRKLHPYETPAIFTLKVENVLTEYMNWIRESV

HAB.2 (used, for example, as N-terminal fusion to SB52 variants, though can be used with other RBS inhibitors) SEQ ID 210 GSMKKVEAIIRPEKLEIVKKALSDAGYVG MTVSEVKGRGVQGGIVERYRGRE YIVDLIPKVKIELVVKEEDVDNVIDIICENARTGNPGDGKIFVIPVERVVRVRT KEEGKEAL

HAB.3 (used, for example, as N-terminal fusion to 5B52 variants, though can be used with other RBS inhibitors) SEQ ID 211 KKIEAIIRPFKLDEVKIALVNAGIVGMTVSEVRCiFGRQKRGSEYTVEFLQKLK LEIVVEDAQVDTVIDKIVAAARTGENGDGKIFVSPVDQTIRIRTGEKNADAI; and

HAB.4 (used, for example, as N-terminal fusion to 5B52 variants, though can be used with other RBS inhibitors) SEQ ID 212 HLTPREFDKLVIHMLSDVALKRKNKGLKLNHPEAVAVLSAYVLDGAREGKT VEEVMDGARSVLKADDVMDGVPDLLPLIQVEAVFSDGSRLVSLHNPI.

### Design Evaluation and Characterization

### Soluble Protein Expression and Purification

Genes were cloned into the bacterial expression vector pET29b between the NdeI and Xhol cut-sites and transformed into BL21Star (Invitrogen). For expression, 10 ml of overnight culture grown in TB II. was used to start a 500 ml culture. Cells were grown for 8 h at 37C, before reducing the temperature to 18° C. for another 14-16 h. We used the Studier autoinduction method with the modification that we replaced yeast extract and peptone with TB II medium. Cells were re-suspended in 35 ml phosphate buffered saline (PBS, 150 mM NaCI and 25 mM phosphate buffer at pH 7.4) and lysed using a microfluidizer or sonicator. Insoluble cell debris was removed by centrifugation for 20 m at 40,000 g. Supernatant was applied to gravity-flow columns containing 2 mL of Ni-NTA for each 500 ml of culture, washed with 50 ml PBS and 50 ml PBS containing 30 mM imidazole. Proteins were eluted with 20 ml of 250 mM imidazole in PBS. If necessary, proteins concentrated to 1.5 mg/ml using a VIVASPIN™ 5 (for the monomers) or 10 kD MWCO (for the trimers) centrifugal concentrator (Sartorius Stedim, Goettingen, Germany) at 4000 g.

Binding Constant Measurements

Titrations were performed on an OCTETRED™ 96 BLI system (ForteBio, Menlo Park, Calif.) using streptavidin-coated biosensors, Sensors were equilibrated for 30 min in PBSTB buffer (PBS, 0.02% Tween 20, 0,01% BSA). For a measurement the following steps were executed at 30° C. while rotating at 1000 rpm: sensors were calibrated again for 60 sec, followed by loading of 15-50 nM biotinylated HA for typically 100 sec, after which we washed the sensors shortly for a 10 sec, before establishing baseline for another 60 sec. Association rates were obtained by incubating each sensor for 60 up to 120 sec in different concentrations of purified designed protein variants spanning the predicted k_{d} for the given HA subtype. Dissociation was measured by incubating between 450-600 sec. All proteins were diluted into 200 μl PBSTB and all washing and calibration steps contained the same buffer.

### Initial Evaluation of Designed Trimers

To evaluate initially how well each of the generated trimer constructs would work as a fusion to the HA binding domain, proteins were expressed in E. coli (as described above and binding constants were obtained via biolayer interferometry instrument OCTET™ (as described above). Data are shown in Table 12.

TABLE 12
Binding to H3 (Hong Kong 1968)
A Initial Screen for off-rates using crude protein extracts

| Sample ID | kdis(1/s) |
|---|---|
| HAA.4 fused to 52solo2 | 3.60E−03 |
| (SEQ ID NO: 16) | |
| HAA.6 fused to 52solo2 | 1.20E−03 |
| HAA.3 fused to 52solo2 | 1.80E−03 |
| HAA.1 fused to 52solo2 | 1.31E−01 |
| HAA.5 fused to 52solo2 | 1.17E−01 |
| HAA.3 fused to 52nc2 | 3.14E−03 |
| (SEQ ID NO: 96) | |
| HAA.1 fused to 52nc2 | 1.47E−03 |

B Measurement of Binding Constants of Ni-NTA purified proteins

| construct | Kd (nM) |
|---|---|
| HAA.5 fused to 52solo2 | 3.737 |
| (SEQ ID NO: 16) | |
| HAA.6 fused to 24alt1 | 4.99 |
| (SEQ ID NO: 125) | |
| HAA.2 fused to 52nc2 | <1 |
| (SEQ ID NO: 96) | |
| HAA.6 fused to 24solo2 | 4.87 |
| HAA.4 fused to 24alt1 | 4.3 |

The data here demonstrates dissociation rates and binding constants of various tamerversions (as indicated) that are binding to the H3 hemagglutinin (Hong Kong 1968). Both off-rates and measured binding constant (Kd) indicates tight binding.

### Comparison of Monomeric Unit Versus Trimeric

To obtain binding constant for both the SB52NC2 variants and the trinierized version, both proteins were independently titrated and measured for binding to H3N2 (A/Hong Kong/1/1968) using biolayer interferometry (BLI). Biotinylated HA was immobilized to the hiosensor and then incubated with either the monomeric SB52nc2 or the trimeric version to obtain the association rates. To measure the dissociation rate, the sensor was incubated in buffer for up to 7 min. Data is shown in FIG. 2 and Table 13. The binding data of the monomeric SB52 variant demonstrate a very fast dissociation rate. On the other hand, the fusion of the SB52 variants to the self-assembling unit HAA.1 resulted in an affinity boost of more than 1000 fold. The trimerization, however, significantly slowed down dissociation from hemagglutinin and thereby “glued” the binding domains to HA resulting in subnanomolar affinity ranges.

TABLE 13
Binding Constants for HAA.1-SB52NC-2 (SEQ ID NO: 96)

| | KD (nM) |
|---|---|
| monovalent SB52NC2 (SEQ ID | |
| NO: 96): | |
| H3N2 A/Hong Kong/1/1968 | 180 |
| 52NC2 - H1 (New Caledonia) | 533.9 |
| 52NC2 - H2 (Adachi) | 289.4 |
| Trimeric HAA.1 fused to 52NC2: | |
| H3N2 A/Hong Kong/1/1968 | 1.5 |
| H1N1 A/New Caledonia/20/1999 | 11.5 |
| H2N2 A/Adachi/2/1957 | 16 |
| H3N2 A/Victoria/361/2011 | 30.9 |

### Trimeric Fusion Increases Breadth of Binding

Binding to H3N2 A/Victoria/361/2011 of monovalent SB52 variant SB52nc2 could not be detected by biolayer interferometry nor by yeast display titrations.

However, binding as trimeric fusions (here to HAA.1) increases the breadth of binding as it dramatically decreases the dissociation of the trimer from HA (FIG. 3).

Optimization of the SB52 HA binding Protein

### Varying Temperature Selections for the Optimization of the Best Variants

To further improve and stabilize the binding protein, all possible amino acid substitutions of the protein variant NC52nc-2 containing cysteines at position 8 and 40 (SB52nc2-8-40 cys) were examined. The single site mutagenesis (SSM) library was screened for mutations that would improve binding over a range of temperature between 22 and 42° C. Yeast cells displaying all possible point mutations were non-stringently selected for binding to hemagglutinin (using 2.5 nM of H1 Solomon Island (2006)) at 22° C., 30° C., 37° C. and 42° C. Substitutions were considered as beneficial to stability if they were enriched consistently across the temperature range.

### Library Constructions

All possible point mutations for each position were generated through overlap PCR using one shorter reverse primer and a longer forward primer that contains the mismatch encoded by NNK. Two fragments for each position were generated, here 2×87 positions. Outer primers for the amplification were colonyF and colonyR; PCRs were performed using 1μl of each primer (20 μM) and 25 ng plasmid DNA of 52NC2-8-40 as a template and 0.125 μl phusion with appropriate buffer and 1.25 μl dNTPs for amplification. The overlap-fusing reaction for each position was performed by combining 1 μl of the two fragments followed by 30 cycles of amplification using the primer colonyF and colonyR primers and the same PCR conditions as the first reaction. All 87 overlap PCR were checked for their success through agarose gel electrophoresis. We combined the reaction by taking 5 μl of each reaction, which was selected for the size through agarose electrophoresis and gel extraction (QIAgen) of the pooled DNA. The extracted DNA was co-transformed with linearized pETCON plasmid (digested with Nhel,XhoI and Sail I) into yeast as above resulting in 10e7 transformants.

### Library Selections

Yeast cells were induced for about 16-18 hat 22° C., washed once with PBS containing 0.1% BSA (PBSF). Library was incubated with 2.5 nM 200 μl 22° C., 30° C., 37° C. and 42° C. for 2 h while rotating, washed once with ice-cold PBSF and incubated at on ice for suspending in 100 μL with nM SAPE and 2 ng/ml anti-Cmyc antibody.

### Library Preparation and Next-Generation Sequencing

Plasmids were extracted as previously described(3). Briefly, around 5×10e7 cells were treated with Zymolase (50 U) in 400 μl Solution buffer 1 (Zymo Research, yeast plasmid miniprep II) and incubated at 37° C. for 4 h and vortexed every hour. Cells were freeze-thawed once and treated as instructed in Zymo kit manual with the exception that lysate was applied to higher-yield columns (QIAgen, plasmid miniprep kit), followed by plasmid elution with 30 μl EB (QIAgen). Possible contaminating genomic DNA was eliminated through digestion with ExoI (NEB) and Lambda exonucelase (NEB)(3). After a QIAgen PCR clean-up step, Illumina flow cell adapters and population specific-barcodes were added through PCR (primer sequences available upon request). PCR product was purified through gel extraction (QIAgen).

The two printer sets have overhangs that add Iliumina sequencing primer binding sites, barcode sequences for each unselected or selected pool and flow cell adaptors to the gene to be sequenced. They additionally add 12 bases at the beginning of the forward and reverse read with alternating basses, ensuring adequate diversity for the Illumina basecalling algorithms. This enabled the DNA pools to be prepared and sequenced in two runs of paired-end 150 bp mode on an illumina MISEQ™ (Illumina, San Diego, Calif.) using a standard MISEQ™ kit and protocols. After quality filtering, sequences were obtained for the starting population and for the selected pool. Each substitution at each position was counted as a single instance, and the ratio of their frequencies of selected versus unselected populations were computed through custom python. For the difference between the different temperature sorts, we used linear regression to identify the best performing substitutions.

The following peptides are single amino acid substitutions that were identified in the temperature selections above, and thus allow the protein to bind better at higher temperatures.

W-64

(SEQ ID NO: 143)

GIVNVPNCNTTKYQQLARTAVAIYNYHEQAHLTFVENLNCKEQLGEGDYY
YITLAATDDAGKKWIYEAKIGVVESAGWTGVEEFKLV
L-64

(SEQ ID NO: 228)

GIVNVPNCNTTKYQQLARTAVAIYNYHEQAHLTFVENLNCKEQLGEGDYY
YITLAATDDAGKKLIYEAKIGVVESAGWTGVEEFKLV
P-29

(SEQ ID NO: 144)

GIVNVPNCNTTKYQQLARTAVAIYNYHEPAHLTFVENLNCKEQLGEGDYY
YTTLAATDDAGKKAIYEAKIGVVESAGWTGVEEFKLV
R-14

(SEQ ID NO: 145)

GIVNVPNCNTTKYRQLARTAVAIYNYHEQAHLTFVENLNCKEQLGEGDYY
YITLAATDDAGKKAIYEAKIGVVESAGWTGVEEFKLV
K-43

(SEQ ID NO: 146)

GIVNVPNCNTTKYQQLARTAVAIYNYHEQAHLTFVENLNCKEKLGEGDYY
YITLAATDDAGKKAIYEAKIGVVESAGWTGVEEFKLV
T-68

(SEQ ID NO: 147)

GIVNVPNCNTTKYQQLARTAVAIYNYHEQAHLTFVENLNCKEQLGEGDYY
YITLAATDDAGKKAIYETKIGVVESAGWTGVEEFKLV
K-11

(SEQ ID NO: 148)

GIVNVPNCNTKKYQQLARTAVAIYNYHEQAHLTFVENLNCKEQLGEGDYY
YITLAATDDAGKKAIYEAKIGVVESAGWTGVEEFKLV
Q-59

(SEQ ID NO: 149)

GIVNVPNCNTTKYQQLARTAVAIYNYHEQAHLTFVENLNCKEQLGEGDYY
YITLAATDQAGKKAIYEAKIGVVESAGWTGVEEFKLV
L-49

(SEQ ID NO: 150)

GIVNVPNCNTTKYQQLARTAVAIYNYHEQAHLTFVENLNCKEQLGEGDLY
YITLAATDDAGKKAIYEAKIGVVESAGWTGVEEFKLV
Y-33

(SEQ ID NO: 151)

GIVNVPNCNTTKYQQLARTAVAIYNYHEQAHLYFVENLNCKEQLGEGDYY
YITLAATDDAGKKAIYEAKIGVVESAGWTGVEEFKLV
N-33

(SEQ ID NO: 152)

GIVNVPNCNTTKYQQLARTAVAIYNYHEQAHLNFVENLNCKEQLGEGDYY
YITLAATDDAGKKAIYEAKIGVVESAGWTGVEEFKLV
W-51

(SEQ ID NO: 153)

GIVNVPNCNTTKYQQLARTAVAIYNYHEQAHLTFVENLNCKEQLGEGDYY
WITLAATDDAGKKAIYEAKIGVVESAGWTGVEEFKLV
A-33

(SEQ ID NO: 154)

GIVNVPNCNTTKYQQLARTAVAIYNYHEQAHLAFVENLNCKEQLGEGDYY
YITLAATDDAGKKAIYEAKIGVVESAGWTGVEEFKLV
I-87

(SEQ ID NO: 155)

GIVNVPNCNTTKYQQLARTAVAIYNYHEQAHLTFVENLNCKEQLGEGDYY
YITLAATDDAGKKAIYEAKIGVVESAGWTGVEEFKLI
A-85

(SEQ ID NO: 156)

GIVNVPNCNTTKYQQLARTAVAIYNYHEQAHLTFVENLNCKEQLGEGDYY
YITLAATDDAGKKAIYEAKIGVVESAGWTGVEEFALV
I-14

(SEQ ID NO: 157)

GIVNVPNCNTTKYIQLARTAVAIYNYHEQAHLTFVENLNCKEQLGEGDYY
YITLAATDDAGKKAIYEAKIGVVESAGWTGVEEFKLV
K-47

(SEQ ID NO: 158)

GIVNVPNCNTTKYQQLARTAVAIYNYHEQAHLTFVENLNCKEQLGEKDYY
YITLAATDDAGKKAIYEAKIGVVESAGWTGVEEFKLV
E-62

(SEQ ID NO: 159)

GIVNVPNCNTTKYQQLARTAVAIYNYHEQAHLTFVENLNCKEQLGEGDYY
YITLAATDDAGEKAIYEAKIGVVESAGWTGVEEFKLV
M-87

(SEQ ID NO: 160)

GIVNVPNCNTTKYQQLARTAVAIYNYHEQAHLTFVENLNCKEQLGEGDYY
YITLAATDDAGKKAIYEAKIGVVESAGWTGVEEFKLV;
D-28

(SEQ ID NO: 161)

GIVNVPNCNTTKYQQLARTAVAIYNYHDQAHLTFVENLNCKEQLGEGDYY
YITLAATDDAGKKAIYEAKIGVVESAGWTGVEEFKLV
S-67

(SEQ ID NO: 162)

GIVNVPNCNTTKYQQLARTAVAIYNYHEQAHLTFVENLNCKEQLGEGDYY
YITLAATDDAGKKAIYSAKIGVVESAGWTGVEEFKLV
E-85

(SEQ ID NO: 163)

GIVNVPNCNTTKYQQLARTAVAIYNYHEQAHLTFVENLNCKEQLGEGDYY
YITLAATDDAGKKAIYEAKIGVVESAGWTGVEEFELV
A-67

(SEQ ID NO: 164)

GIVNVPNCNTTKYQQLARTAVAIYNYHEQAHLTFVENLNCKEQLGEGDYY
YITLAATDDAGKKAIYAAKIGVVESAGWTGVEEFKLV
H-36

(SEQ ID NO: 165)

GIVNVPNCNTTKYQQLARTAVAIYNYHEQAHLTFVHNLNCKEQLGEGDYY
YITLAATDDAGKKAIYEAKIGVVESAGWTGVEEFKLV
L-81

(SEQ ID NO: 166)

GIVNVPNCNTTKYQQLARTAVAIYNYHEQAHLTFVENLNCKEQLGEGDYY
YITLAATDDAGKKAIYEAKIGVVESAGWTGLEEFKLV
E-30

(SEQ ID NO: 167)

GIVNVPNCNTTKYQQLARTAVAIYNYHEQEHLTFVENLNCKEQLGEGDYY
YITLAATDDAGKKAIYEAKIGVVESAGWTGVEEFKLV
P-11

(SEQ ID NO: 168)

GIVNVPNCNTPKYQQLARTAVAIYNYHEQAHLTFVENLNCKEQLGEGDYY
YITLAATDDAGKKAIYEAKIGVVESAGWTGVEEFKLV
M-64

(SEQ ID NO: 169)

GIVNVPNCNTTKYQQLARTAVAIYNYHEQAHLTFVENLNCKEQLGEGDYY
YITLAATDDAGKKMIYEAKIGVVESAGWTGVEEFKLV
S-64

(SEQ ID NO: 170)

GIVNVPNCNTTKYQQLARTAVAIYNYHEQAHLTFVENLNCKEQLGEGDYY
YITLAATDDAGKKSIYEAKIGVVESAGWTGVEEFKLV
K-28

(SEQ ID NO: 171)

GIVNVPNCNTTKYQQLARTAVAIYNYHKQAHLTFVENLNCKEQLGEGDYY
YITLAATDDAGKKAIYEAKIGVVESAGWTGVEEFKLV
S-22

(SEQ ID NO: 172)

GIVNVPNCNTTKYQQLARTAVSIYNYHEQAHLTFVENLNCKEQLGEGDYY
YITLAATDDAGKKAIYEAKIGVVESAGWTGVEEFKLV
D-27

(SEQ ID NO: 173)

GIVNVPNCNTTKYQQLARTAVAIYNYDEQAHLTFVENLNCKEQLGEGDYY
YITLAATDDAGKKAIYEAKIGVVESAGWTGVEEFKLV
L-33

(SEQ ID NO: 174)

GIVNVPNCNTTKYQQLARTAVAIYNYHEQAHLLFVENLNCKEQLGEGDYY
YITLAATDDAGKKAIYEAKIGVVESAGWTGVEEFKLV
I-33

(SEQ ID NO: 175)

GIVNVPNCNTTKYQQLARTAVAIYNYHEQAHLIFVENLNCKEQLGEGDYY
YITLAATDDAGKKAIYEAKIGVVESAGWTGVEEFKLV
W-28

(SEQ ID NO: 176)

GIVNVPNCNTTKYQQLARTAVAIYNYHWQAHLTFVENLNCKEQLGEGDYY
YITLAATDDAGKKAIYEAKIGVVESAGWTGVEEFKLV
Q-64

(SEQ ID NO: 177)

GIVNVPNCNTTKYQQLARTAVAIYNYHEQAHLTFVENLNCKEQLGEGDYY
YITLAATDDAGKKQIYEAKIGVVESAGWTGVEEFKLV
Y-67

(SEQ ID NO: 178)

GIVNVPNCNTTKYQQLARTAVAIYNYHEQAHLTFVENLNCKEQLGEGDYY
YITLAATDDAGKKAIYYAKIGVVESAGWTGVEEFKLV
Y-22

(SEQ ID NO: 179)

GIVNVPNCNTTKYQQLARTAVYIYNYHEQAHLTFVENLNCKEQLGEGDYY
YITLAATDDAGKKAIYEAKIGVVESAGWTGVEEFKLV
G-64

(SEQ ID NO: 180)

GIVNVPNCNTTKYQQLARTAVAIYNYHEQAHLTFVENLNCKEQLGEGDYY
YITLAATDDAGKKGIYEAKIGVVESAGWTGVEEFKLV
W-67

(SEQ ID NO: 181)

GIVNVPNCNTTKYQQLARTAVAIYNYHEQAHLTFVENLNCKEQLGEGDYY
YITLAATDDAGKKAIYWAKIGVVESAGWTGVEEFKLV
M-67

(SEQ ID NO: 182)

GIVNVPNCNTTKYQQLARTAVAIYNYHEQAHLTFVENLNCKEQLGEGDYY
YITLAATDDAGKKAIYMAKIGVVESAGWTGVEEFKLV
D-29

(SEQ ID NO: 183)

GIVNVPNCNTTKYQQLARTAVAIYNYHEDAHLTFVENLNCKEQLGEGDYY
YITLAATDDAGKKAIYEAKIGVVESAGWTGVEEFKLV
H-33

(SEQ ID NO: 184)

GIVNVPNCNTTKYQQLARTAVAIYNYHEQAHLHFVENLNCKEQLGEGDYY
YITLAATDDAGKKAIYEAKIGVVESAGWTGVEEFKLV
D-85

(SEQ ID NO: 185)

GIVNVPNCNTTKYQQLARTAVAIYNYHEQAHLTFVENLNCKEQLGEGDYY
YITLAATDDAGKKAIYEAKIGVVESAGWTGVEEFDLV
M-24

(SEQ ID NO: 186)

GIVNVPNCNTTKYQQLARTAVAIMNYHEQAHLTFVENLNCKEQLGEGDYY
YITLAATDDAGKKAIYEAKIGVVESAGWTGVEEFKLV
A-64

(SEQ ID NO: 187)

GIVNVPNCNTTKYQQLARTAVAIYNYHEQAHLTFVENLNCKEQLGEGDYY
YITLAATDDAGKKAIYEAKIGVVESAGWTGVEEFKLV
Y-49

(SEQ ID NO: 188)

GIVNVPNCNTTKYQQLARTAVAIYNYHEQAHLTFVENLNCKEQLGEGDYY
YITLAATDDAGKKAIYEAKIGVVESAGWTGVEEFKLV
L-79

(SEQ ID NO: 189)

GIVNVPNCNTTKYQQLARTAVAIYNYHEQAHLTFVENLNCKEQLGEGDYY
YITLAATDDAGKKAIYEAKIGVVESAGWLGVEEFKLV
S-33

(SEQ ID NO: 190)

GIVNVPNCNTTKYQQLARTAVAIYNYHEQAHLSFVENLNCKEQLGEGDYY
YITLAATDDAGKKAIYEAKIGVVESAGWTGVEEFKLV
W-24

(SEQ ID NO: 191)

GIVNVPNCNTTKYQQLARTAVAIWNYHEQAHLTFVENLNCKEQLGEGDYY
YITLAATDDAGKKAIYEAKIGVVESAGWTGVEEFKLV
I-24

(SEQ ID NO: 192)

GIVNVPNCNTTKYQQLARTAVAIINYHEQAHLTFVENLNCKEQLGEGDYY
YITLAATDDAGKKAIYEAKIGVVESAGWTGVEEFKLV
Q-19

(SEQ ID NO: 193)

GIVNVPNCNTTKYQQLARQAVAIYNYHEQAHLTFVENLNCKEQLGEGDYY
YITLAATDDAGKKAIYEAKIGVVESAGWTGVEEFKLV
V-24

(SEQ ID NO: 194)

GIVNVPNCNTTKYQQLARTAVAIVNYHEQAHLTFVENLNCKEQLGEGDYY
YITLAATDDAGKKAIYEAKIGVVESAGWTGVEEFKLV
F-33

(SEQ ID NO: 195)

GIVNVPNCNTTKYQQLARTAVAIYNYHEQAHLFFVENLNCKEQLGEGDYY
YITLAATDDAGKKAIYEAKIGVVESAGWTGVEEFKLV
E-29

(SEQ ID NO: 229)

GIVNVPNCNTTKYQQLARTAVAIYNYHEEAHLTFVENLNCKEQLGEGDYY
YITLAATDDAGKKAIYEAKIGVVESAGWTGVEEFKLV
M-33

(SEQ ID NO: 196)

GIVNVPNCNTTKYQQLARTAVAIYNYHEQAHLMFVENLNCKEQLGEGDYY
YITLAATDDAGKKAIYEAKIGVVESAGWTGVEEFKLV
K-15

(SEQ ID NO: 197)

GIVNVPNCNTTKYQKLARTAVAIYNYHEQAHLTFVENLNCKEQLGEGDYY
YITLAATDDAGKKAIYEAKIGVVESAGWTGVEEFKLV
V-33

(SEQ ID NO: 198)

GIVNVPNCNTTKYQQLARTAVAIYNYHEQAHLVFVENLNCKEQLGEGDYY
YITLAATDDAGKKAIYEAKIGVVESAGWTGVEEFKLV
Q-36

(SEQ ID NO: 199)

GIVNVPNCNTTKYQQLARTAVAIYNYHEQAHLTFVQNLNCKEQLGEGDYY
YITLAATDDAGKKAIYEAKIGVVESAGWTGVEEFKLV
F-67

(SEQ ID NO: 77)

GIVNVPNCNTTKYQQLARTAVAIYNYHEQAHLTFVENLNCKEQLGEGDYY
YITLAATDDAGKKAIYFAKIGVVESAGWTGVEEFKLV
L-70

(SEQ ID NO: 79)

GIVNVPNCNTTKYQQLARTAVAIYNYHEQAHLTFVENLNCKEQLGEGDYY
YITLAATDDAGKKAIYEAKLGVVESAGWTGVEEFKLV
Y-28

(SEQ ID NO: 230)

GIVNVPNCNTTKYQQLARTAVAIYNYHYQAHLTFVENLNCKEQLGEGDYY
YITLAATDDAGKKAIYEAKIGVVESAGWTGVEEFKLV
R-15

(SEQ ID NO: 231)

GIVNVPNCNTTKYQRLARTAVAIYNYHEQAHLTFVENLNCKEQLGEGDYY
YITLAATDDAGKKAIYEAKIGVVESAGWTGVEEFKLV
Q-33

(SEQ ID NO: 232)

GIVNVPNCNTTKYQQLARTAVAIYNYHEQAHLQFVENLNCKEQLGEGDYY
YITLAATDDAGKKAIYEAKIGVVESAGWTGVEEFKLV
R-33

(SEQ ID NO: 233)

GIVNVPNCNTTKYQQLARTAVAIYNYHEQAHLRFVENLNCKEQLGEGDYY
YITLAATDDAGKKAIYEAKIGVVESAGWTGVEEFKLV
V-21

(SEQ ID NO: 234)

GIVNVPNCNTTKYQQLARTAVAIYNYHEQAHLTFVENLNCKEQLGEGDYY
YITLAATDDAGKKAIYEAKIGVVESAGWTGVEEFKLV
S-76

(SEQ ID NO: 235)

GIVNVPNCNTTKYQQLARTAVAIYNYHEQAHLTFVENLNCKEQLGEGDYY
YITLAATDDAGKKAIYEAKIGVVESSGWTGVEEFKLV
W-49

(SEQ ID NO: 236)

GIVNVPNCNTTKYQQLARTAVAIYNYHEQAHLTFVENLNCKEQLGEGDWY
YITLAATDDAGKKAIYEAKIGVVESAGWTGVEEFKLV
E-28

(SEQ ID NO: 237)

GIVNVPNCNTTKYQQLARTAVAIYNYHEQAHLTFVENLNCKEQLGEGDYY
YITLAATDDAGKKAIYEAKIGVVESAGWTGVEEFKLV
H-22

(SEQ ID NO: 238)

GIVNVPNCNTTKYQQLARTAVHIYNYHEQAHLTFVENLNCKEQLGEGDYY
YITLAATDDAGKKAIYEAKIGVVESAGWTGVEEFKLV
K-33

(SEQ ID NO: 239)

GIVNVPNCNTTKYQQLARTAVAIYNYHEQAHLKFVENLNCKEQLGEGDYY
YITLAATDDAGKKAIYEAKIGVVESAGWTGVEEFKLV
G-17

(SEQ ID NO: 240)

GIVNVPNCNTTKYQQLGRTAVAIYNYHEQAHLTFVENLNCKEQLGEGDYY
YITLAATDDAGKKAIYEAKIGVVESAGWTGVEEFKLV
T-33

(SEQ ID NO: 241)

GIVNVPNCNTTKYQQLARTAVAIYNYHEQAHLTFVENLNCKEQLGEGDYY
YITLAATDDAGKKAIYEAKIGVVESAGWTGVEEFKLV
T-19

(SEQ ID NO: 242)

GIVNVPNCNTTKYQQLARTAVAIYNYHEQAHLTFVENLNCKEQLGEGDYY
YITLAATDDAGKKAIYEAKIGVVESAGWTGVEEFKLV

### Generation of Combinatorial Libraries

To find ideal combination of the substitutions that enabled better binding at higher temperatures, we generated a combinatorial library in which we allowed 11 positions to change accordingly to the previously identified substitutions. The gene was assembled recursively using ultramers (IDT) containing degenerate codons.

TABLE 14

| | Amino |
|---|---|
| Position | Acid |
| 9 | N |
| 19 | ST |
| 11 | VI |
| 24 | YVWMI |
| 28 | QKED |
| 29 | PED |
| 33 | TRKQ |
| 49 | MLY |
| 64 | SA |
| 68 | KAY |
| 70 | IL |

The following peptide sequences were identified after 5 rounds of sorting at 37° C. Genes were cloned into pET29b and expressed in E. coli. Hsc1 expressed significantly higher than the other identified clones:

Hsc1

(SEQ ID NO: 135)

GIVNVPNCNTTKYQQLARTAVAIYNYHEQAHLTFVENLNCKEQLGEGDYY
YITLAATDDAGKKAIYEAKIGVVESAGWTGVEEFKLV
Hsc3

(SEQ ID NO: 136)

GIVNVPNCNTTKYQQLARTAIAIMNYHQQAHLTFVENLNCKEQLGEGDYY
YITLAATDDAGKKAIYEAKIGVVESAGWTGVEEFKLV
Hsc12

(SEQ ID NO: 137)

GIVNVPNCNTTKYQQLARTAIAIVNCHEHAHLTFVENLNCKEQLGEGDIY
YITLAATDDAGKKSIYEAKIGVVESAGWTGVEEFKLV
HscA

(SEQ ID NO: 138)

GIVNVPNCNTTYKYQQLARTAVAIYNYHKPAHLTFVENLNCKEQLGEGDI
YYITLAATDDAGKKSIYSAKIGVVESAGWTGVEEFKLV
HscB

(SEQ ID NO: 139)

GIVNVPNCNTTKYQQLARTAVAIYNYHNHAHLTFVENLNCKEQLGEGDLY
YITLAATDDAGKKAIYTAKIGVVESAGWTGVEEFKLV

### Determination of Binding Breadth for the Variant

To evaluate the binding breadth of Hsc1 (SEQ ID NO:135) as a trimer, we measured its binding of when fused to HAA.1c (SEQ ID NO:227) via the triLong linker (SEQ ID NO: 352) to a variety of flu strains as summarized in table 15. The protein was expressed in E. coli and purified using a Ni-NTA resin and size exclusion chromatography. Binding against a variety of strains was tested using biolayer interferonietry as described above; the data is summarized under Table 15. The variant has a broad binding spectrum which binds to strains from both group I and II Influenza A viruses. However, as expected, a few strains could not be targeted. In the subsection below we address further sequences that target the remaining strains.

TABLE 15

| | | Kd |
|---|---|---|
| Strain | | (nM) |
| A/New Caledonia/20/1999 | H1 | <1 |
| A/Solomon Islands/3/2006 | H1 | <1 |
| A/Texas/36/1991 | H1 | — |
| A/Beijing/262/1995 | H1 | 2.8 |
| A/USSR/90/1977 | H1 | — |
| A/Adachi/2/1957 | H2 | <1 |
| A/Japan/305/1957 | H2 | 4.8 |
| A/Panama/2007/1999 | H3 | 56.5 |
| A/Hong Kong/1/1968 | H3 | <1 |
| A/Victoria/361/2011 | H3 | <1 |
| A/Shangdong/9/1993 | H3 | — |
| A/Moscow/10/1999 | H3 | — |
| A/Perth/16/2009 | H3 | — |
| A/Vietnam/1203/2004 | H5 | — |
| A/Shanghai/2/2013 | H7 | — |
| A/Netherlands/219/2003 | H7 | — |
| A/turkey/Wisconsin/1/1966 | H9 | 9.4 |
| A/chicken/Germany/N/1949 | H10 | — |
| A/duck/England/1/1956 | H11 | — |
| A/duck/Alberta/60/1976 | H12 | 9.2 |
| A/gull/Maryland/704/1977 | H13 | 681.0 |
| A/shearwater/Western Australia/2576/1979 | H15 | — |
| A/black-headed gull/Sweden/4/1999 | H16 | — |

### Further Modifications for Stabilization

For stabilization and solubility, several further modifications were undertaken: first we introduced an alternative disulfide bridge between residues 33-57. Secondly, we tested different loop variations of loop 2 (first loop between the first two beta-sheets) between positions 44-46.

Hsc1-3357

(SEQ ID NO: 140)

GIVNVPNPNTTKYQQLARTAVAIYNYHEQAHLCFVENLNVKEQLGEGDYY
YITLAACDDAGKKAIYEAKIGVVESAGWTGVEEFKLV
Hsc1-EA

(SEQ ID NO: 141)

GIVNVPNCNTTKYQQLARTAVAIYNYHEQAHLTFVENLNCKEQGNYYYIT
LAATDDAGKKAIYEAKIGVVESAGWTGVEEFKLV
Hsc1-3357EA

(SEQ ID NO: 142)

GIVNVPNPNTTKYQQLARTAVAIYNYHEQAHLCFVENLNVKEQGNYYYIT
LAACDDAGKKAIYEAKIGVVESAGWTGVEEFKLV

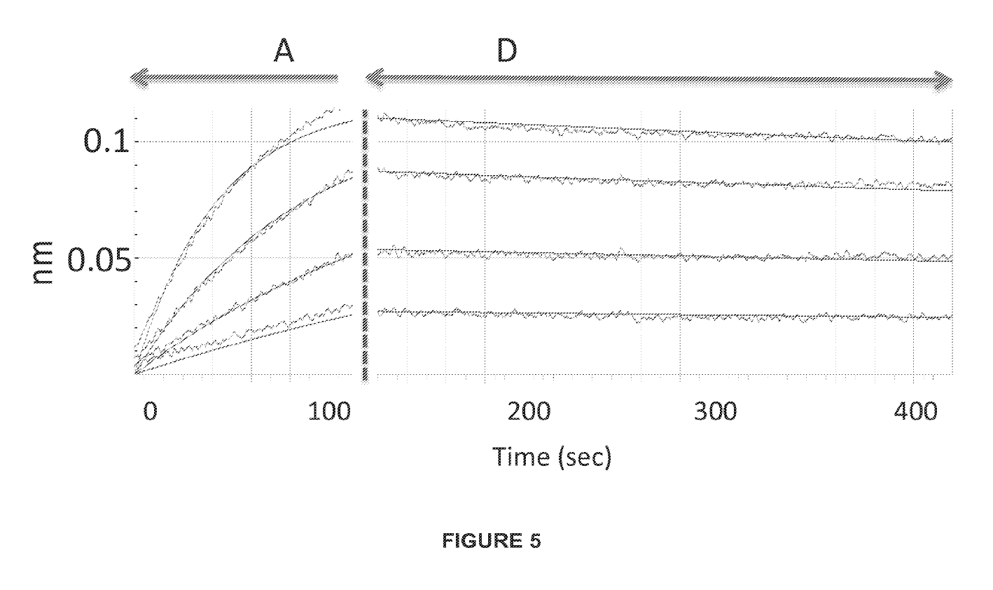

Hsc1-3357EA and Hsci -EA have a truncation of 3 residues after position 43. The monomeric version binds worse than the longer Hsc1 variant, but as soon as it is trimerized it binds extremely tightly and its stability is improved (FIG. 5).

### Rational Design of Variants to Target an Extensive Set of Influenza Strains

As expected, we could not identify a single clone that could target all strains. This is due to the fact that the binding site has contacts to a few surface residues of HA that are part of antigenic drifts and are changing over time. Charge inversions and conversions of small residues into large and vice versa can be found when analyzing 10,000 non-redundant influenza A strains. Therefore we designed a variety of sequences that have chemical complementary to the sequence changes in the HA molecule in the area of the binding epitope the SB52 variants. As a starting point, we utilized our design model.

Additionally, we screened a combinatorial library against 5 strains from the H1, H2 and H3 subtypes (H1N1 A/Singapore/6/1986, H1N1 A/Solomon Islands/3/2006, H2N2 A/Adachi/2/1957, H3N2 A/Hong Kong/1/1968 and H3N2 A/Victoria/361/2011) and sequenced thousands of the variants enriched after either 1 or 2 rounds of selections using a MISEQ™ (Tilumina and protocol as above). Sequences were analyzed in context of their specificities towards the 5 strains and re-designed to (1) accommodate the most stable sequence composition as we identified after the differential temperature selection and combinatorial library screen at a higher temperature. In short, interface residues of Hsc 1 with HA (as modeled) were re-designed. (2) Based on these sequences we identified positions that accommodated the changing chemical complementarity of the HA head region. Therefore, we designed sequences that would be the chemical counterpart to address the chemical complexity of HA. To realize this, we generated a sequence profile of the head-region epitope for 10,000 non-redundant H1 and H3 strains obtained from the Influenza Research Database (IRD) and re-designed the contact residues of the Hsc1 variant to reflect this chemical diversity. Designed sequences are listed below.

h2_perm9

(SEQ ID NO: 243)

GIVNVPNCNTTKYQDLAQTAVAIYNYHEQAHLTFVENLNCKEQINGYDYY
YITLAATDDAGKKAIYEAKIGVVESAGWFGVEEFKLV
gen_perm13

(SEQ ID NO: 244)

GIVNVPNCNTTKYQQLAQTAVAIYNYHEQAHLTFVENLNCKEQLGEGDYY
YITLAATDDAGKKAIYEAKIGVVESAGWTGVEEFKLV
gen_perm14

(SEQ ID NO: 245)

GIVNVPNCNTTKYQQLAQTAVAIYNYHEQAHLTFVENLNCKEQLGEGDRY
YITLAATDDAGKKAIYEAKIGVVESAGWTGVEEFKLV
panb_perm9

(SEQ ID NO: 246)

GIVNVPNCNTTKYQQLAQTAVAIYNYHEQAHLTFVENLNCKEQLGGPDRY
YITLAATDDAGKKAIYEAKIGVVEPAGWVAVMEFKLV
h2_perm2

(SEQ ID NO: 247)

GIVNVPNCNTTKYQDLAQTAVAIYNYHEQAHLTFVENLNCKEQLGEGDYY
YITLAATDDAGKKAIYEAKIGVVESAGWKGVEEFKLV
pan_perm6

(SEQ ID NO: 248)

GIVNVPNCNTTKYQQLARTAVAIYNYHEQAHLTFVENLNCKEQLGGPDRY
YITLAATDDAGKKAIYEAKIGVVEPAGWVAVEEFKLV
combs5

(SEQ ID NO: 249)

GIVNVPNCNTTKYQQLARSAVAIYNYHEQAHLTFVENLNCKEQLNDGIYY
YITLAATDDAGKKAIYEAKIGVVESAGWIGVEEFKLV
pr_gen_pan6

(SEQ ID NO: 250)

GIVNVPNCNTTKYQRLADRAVAIYNYHEQAHLTFVENLNCKEQVGEGVYY
YITLAATDDAGKKAIYEAKIGVVEPAGWVAVEEFKLV
panb_perm13

(SEQ ID NO: 251)

GIVNVPNCNTTKYQQLAQRAVAIYNYHEQAHLTFVENLNCKEQWGLGIRY
YITLAATDDAGKKAIYEAKIGVVEPAGWVAVMEFKLV
panb_perm3

(SEQ ID NO: 252)

GIVNVPNCNTTKYQQLARTAVAIYNYHEQAHLTFVENLNCKEQLGGPDRY
YITLAATDDAGKKAIYEAKIGVVEPAGWVAVMEFKLV
gen_perm29

(SEQ ID NO: 253)

GIVNVPNCNTTKYQQLAQRAVAIYNYHEQAHLTFVENLNCKEQINGYDYY
YITLAATDDAGKKAIYEAKIGVVESAGWTGVEEFKLV
panb_perm14

(SEQ ID NO: 254)

GIVNVPNCNTTKYQQLAQRAVAIYNYHEQAHLTFVENLNCKEQWGLGIMY
YITLAATDDAGKKAIYEAKIGVVEPAGWVAVMEFKLV
pan_perm8

(SEQ ID NO: 255)

GIVNVPNCNTTKYQQLAQTAVAIYNYHEQAHLTFVENLNCKEQWGLGIMY
YITLAATDDAGKKAIYEAKIGVVEPAGWVAVEEFKLV
pan_perm14

(SEQ ID NO: 256)

GIVNVPNCNTTKYQQLAQRAVAIYNYHEQAHLTFVENLNCKEQWGLGIMY
YITLAATDDAGKKAIYEAKIGVVEPAGWVAVEEFKLV
panb_perm8

(SEQ ID NO: 257)

GIVNVPNCNTTKYQQLAQTAVAIYNYHEQAHLTFVENLNCKEQWGLGIMY
YITLAATDDAGKKAIYEAKIGVVEPAGWVAVMEFKLV
h2_perm1

(SEQ ID NO: 258)

GIVNVFNCNTTKYQDLAQTAVAIYNYHEQAHLTFVENLNCKEQLGEGDYY
YITLAATDDAGKKAIYEAKIGVVESAGWFGVEEFKLV
gen_perm20

(SEQ ID NO: 259)

GIVNVPNCNTTKYQQLAQDAVAIYNYHEQAHLTFVENLNCKEQLGEGDRY
YITLAATDDAGKKAIYEAKIGVVESAGWTGVEEFKLV
h2_perm6

(SEQ ID NO: 260)

GIVNVPNCNTTKYQDLAQTAVAIYNYHEQAHLTFVENLNCKEQLGDGVYY
YITLAATDDAGKKAIYEAKIGVVESAGWKGVEEFKLV
trans_alt3

(SEQ ID NO: 261)

GIVNVPNCNTTKYQQLARSAVAIYNYHEQAHLTFVENLNCKEQVSYGAYY
YITLAATDDAGKKAIYEAKIGVVESAGWVGVEEFKLV
h2t3

(SEQ ID NO: 262)

GIVNVPNCNTTKYQQLARSAVAIYNYHEQAHLTFVENLNCKEQTAKGNYY
YITLAATDDAGKKAIYEAKIGVVESAGWVGVEEFKLV
h2_perm5

(SEQ ID NO: 263)

GIVNVPNCNTTKYQDLAQTAVAIYNYHEQAHLTFVENLNCKEQLGDGVYY
YITLAATDDAGKKAIYEAKIGVVESAGWFGVEEFKLV
singt2

(SEQ ID NO: 264)

GIVNVPNCNTTKYQQLARSAVAIYNYHEQAHLTFVENLNCKEQAGLGGYY
YITLAATDDAGKKAIYEAKIGVVESAGWVAVGEFKLV
gen_perm25

(SEQ ID NO: 265)

GIVNVPNCNTTKYQQLAQRAVAIYNYHEQAHLTFVENLNCKEQLGEGDYY
YITLAATDDAGKKAIYEAKIGVVESAGWTGVEEFKLV
hkt3

(SEQ ID NO: 266)

GIVNVPNCNTTKYQQLARSAVAIYNYHEQAHLTFVENLNCKEQVGDGDYY
YITLAATDDAGKKAIYEAKTGVVESAGWVGVNEFKLV
trans_solo1

(SEQ ID NO: 267)

GIVNVPNCNTTKYQQLARSAVAIYNYHEQAHLTFVENLNCKEQVGGMNYY
YITLAATDDAGKKAIYEAKIGVVESAGWKGVEEFKLV
pan_perm15

(SEQ ID NO: 268)

GIVNVPNCNTTKYQQLAQRAVAIYNYHEQAHLTFVENLNCKEQLGGPDRY
YITLAATDDAGKKAIYEAKIGVVEPAGWVAVEEFKLV
gen_perm3

(SEQ ID NO: 269)

GIVNVPNCNTTKYQQLARTAVAIYNYHEQAHLTFVENLNCKEQLGDGVYY
YITLAATDDAGKKAIYEAKIGVVESAGWTGVEEFKLV
pan_perm7

(SEQ ID NO: 270)

GIVNVPNCNTTKYQQLAQTAVAIYNYHEQAHLTFVENLNCKEQWGLGIRY
YITLAATDDAGKKAIYEAKIGVVEPAGWVAVEEFKLV
combs17

(SEQ ID NO: 271)

GIVNVPNCNTTKYQQLARRAVAIYNYHEQAHLTFVENLNCKEQAGIGGYY
YITLAATDDAGKKAIYEAKIGVVESAGWVAVGEFKLV
combs8

(SEQ ID NO: 272)

GIVNVPNCNTTKYQQLARIAVAIYNYHEQAHLTFVENLNCKEQIGEGNYY
YITLAATDDAGKKAIYEAKIGVVESAGWIGVEEFKLV
pr_gen_pan2

(SEQ ID NO: 273)

GIVNVPNCNTTKYQRLADRAVAIYNYHEQAHLTFVENLNCKEQLGEGDRY
YITLAATDDAGKKAIYEAKIGVVEPAGWVAVEEFKLV
gen_perm27

(SEQ ID NO: 274)

GIVNTVPNCNTTKYQQLAQRAVAIYNYHEQAHLTFVENLNCKEQLGDGVY
YYITLAATDDAGKKAIYEAKIGVVESAGWTGVEEFKLV
gen_perm4

(SEQ ID NO: 275)

GIVNVPNCNTTKYQQLARTAVAIYNYHEQAHLTFVENLNCKEQLTGYDYY
YITLAATDDAGKKAIYEAKIGVVESAGWTGVEEFKLV
h2_perm8

(SEQ ID NO: 276)

GIVNVPNCNTTKYQDLAQTAVAIYNYHEQAHLTFVENLNCKEQLTGYDYY
YITLAATDDAGKKAIYEAKIGVVESAGWKGVEEFKLV
gen_perm28

(SEQ ID NO: 277)

GIVNVPNCNTTKYQQLAQRAVAIYNYHEQAHLTFVENLNCKEQLTGYDYY
YITLAATDDAGKKAIYEAKIGVVESAGWTGVEEFKLV
vt1

(SEQ ID NO: 278)

GIVNVPNCNTTKYQQLARRAVAIYNYHEQAHLTFVENLNCKEQINGYDYY
YITLAATDDAGKKAIYEAKIGVVESAGWEVVGEFKLV
pan_perm9

(SEQ ID NO: 279)

GIVNVPNCNTTKYQQLAQTAVAIYNYHEQAHLTFVENLNCKEQLGGPDRY
YITLAATDDAGKKAIYEAKIGVVEPAGWVAVEEFKLV
gen_perm30

(SEQ ID NO: 280)

GIVNVPNCNTTKYQQLAQRAVAIYNYHEQAHLTFVENLNCKEQVGEGVYY
YITLAATDDAGKKAIYEAKIGVVESAGWTGVEEFKLV
pr_gen_pan3

(SEQ ID NO: 281)

GIVNVPNCNTTKYQRLADRAVAIYNYHEQAHLTFVENLNCKEQLGDGVYY
YITLAATDDAGKKAIYEAKIGVVEPAGWVAVEEFKLV
panb_perm15

(SEQ ID NO: 282)

GIVNVPNCNTTKYQQLAQRAVAIYNYHEQAHLTFVENLNCKEQLGGPDRY
YITLAATDDAGKKAIYEAKIGVVEPAGWAVMEFKLV
pan_perm11

(SEQ ID NO: 283)

GIVNVPNCNTTKYQQLAQDAVAIYNYHEQAHLTFVENLNCKEQWGLGIMY
YITLAATDDAGKKAIYEAKIGVVEPAGWVAVEEFKLV
combs15

(SEQ ID NO: 284)

GIVNVPNCNTTKYQQLARIAVAIYNYHEQAHLTFVENLNCKEQVSGYDYY
YITLAATDDAGKKAIYEAKIGVVESAGWTGVEEFKLV
pr_gen_gen2

(SEQ ID NO: 285)

GIVNVPNCNTTKYQRLADRAVAIYNYHEQAHLTFVENLNCKEQLGEGDRY
YITLAATDDAGKKAIYEAKIGVVESAGWTGVEEFKLV
panb_perm12

(SEQ ID NO: 286)

GIVNVPNCNTTKYQQLAQDAVAIYNYHEQAHLTFVENLNCKEQLGGPDRY
YITLAATDDAGKKAIYEAKIGVVEPAGWVAVMEFKLV
gen_perm26

(SEQ ID NO: 287)

GIVNVPNCNTTKYQQLAQRAVAIYNYHEQAHLTFVENLNCKEQLGEGDRY
YITLAATDDAGKKAIYEAKIGVVESAGWTGVEEFKLV
pr_gen_gen6

(SEQ ID NO: 288)

GIVNVPNCNTTKYQRLADRAVAIYNYHEQAHLTFVENLNCKEQVGEGVYY
YITLAATDDAGKKAIYEAKTGVVESAGWTGVEEFKLV
trans_alt4

(SEQ ID NO: 289)

GIVNVPNCNTTKYQQLARSAVAIYNYHEQAHLTFVENLNCKEQIDYGAYY
YITLAATDDAGKKAIYEAKIGVVESAGWIGVEEFKLV
h2t1

(SEQ ID NO: 290)

GIVNVPNCNTTKYQQLARIAVAIYNYHEQAHLTFVENLNCKEQLAVGDYY
YITLAATDDAGKKAIYEAKIGVVESAGWIGVEEFKLV
panb_perm7

(SEQ ID NO: 291)

GIVNVPNCNTTKYQQLAQTAVAIYNYHEQAHLTFVENLNCKEQWGLGIRY
YITLAATDDAGKKAIYEAKIGVVEPAGWVAVMEFKLV
trans_solo2

(SEQ ID NO: 292)

GIVNVPNCNTTKYQQLARSAVAIYNYHEQAHLTFVENLNCKEQIAGKTYY
YITLAATDDAGKKAIYEAKIGVVESAGWEGVEEFKLV
combs12

(SEQ ID NO: 293)

GIVNVPNCNTTKYQQLARSAVAIYNYHEQAHLTFVENLNCKEQSSGYSYY
YITLAATDDAGKKAIYEAKIGVVESAGWEAVTEFKLV
pan_perm3

(SEQ ID NO: 294)

GIVNVPNCNTTKYQQLARTAVAIYNYHEQAHLTFVENLNCKEQWGLGIRY
YITLAATDDAGKKAIYEAKIGVVEPAGWVAVEEFKLV
pan_perm13

(SEQ ID NO: 295)

GIVNVPNCNTTKYQQLAQRAVAIYNYHEQAHLTFVENLNCKEQWGLGIRY
YITLAATDDAGKKAIYEAKIGVVEPAGWVAVEEFKLV
pr_gen_gen4

(SEQ ID NO: 296)

GIVNVPNCNTTKYQRLADRAVAIYNYHEQAHLTFVENLNCKEQLTGYDYY
YITLAATDDAGKKAIYEAKIGVVESAGWTGVEEFKLV
vt3

(SEQ ID NO: 297)

GIVNVPNCNTTKYQQLARSAVAIYHEQAHLTFVENLNCKEQLTGFDYYYI
TLAATDDAGKKAIYEAKIGVVESAGWEGVNEFKLV
combs13

(SEQ ID NO: 298)

GIVNVPNCNTTKYQQLARSAVAIYNYHEQAHLTFVENLNCKEQLGGYSYY
YITLAATDDAGKKAIYEAKIGVVESAGWVGVGEFKLV
combs24

(SEQ ID NO: 299)

GIVNVPNCNTTKYQQLARSAVAIYNYHEQAHLTFVENLNCKEQINDGVYY
YITLAATDDAGKKAIYEAKIGVVESAGWVGVEEFKLV
combs3

(SEQ ID NO: 300)

GIVNVPNCNTTKYQQLARSAVAIYNYHEQAHLTFVENLNCKEQSDLGSYY
YITLAATDDAGKKAIYEAKIGVVESAGWIGVEEFKLV
combs16

(SEQ ID NO: 301)

GIVNVPNCNTTKYQQLARRAVAIYNYHEQAHLTFVENLNCKEQTGLGIYY
YITLAATDDAGKKAIYEAKIGVVESAGWVAVGEFKLV
h2_perm10

(SEQ ID NO: 302)

GIVNVPNCNTTKYQDLAQTAVAIYNYHEQAHLTFVENLNCKEQINGYDYY
YITLAATDDAGKKAIYEAKIGVVESAGWKGVEEFKLV
combs4

(SEQ ID NO: 303)

GIVNVPNCNTTKYQQLARSAVAIYNHEQAHLTFVENLNCKEQTGGPAYYY
ITLAATDDAGKKAIYEAKIGVVESAGWIGVEEFKLV
panb_perm11

(SEQ ID NO: 304)

GIVNVPNCNTTKYQQLAQDAVAIYNYHEQAHLTFVENLNCKEQWGLGIMY
YITLAATDDAGKKAIYEAKIGVVEPAGWVAMEFKLV
gen_perm24

(SEQ ID NO: 305)

GIVNVPNCNTTKYQQLAQDAVAIYNYHEQAHLTFVENLNCKEQVGEGVYY
YITLAATDDAGKKAIYEAKIGVVESAGWTGVEEFKLV
gen_perm5

(SEQ ID NO: 306)

GIVNVPNCNTTKYQQLARTAVAIYNYHEQAHLTFVENLNCKEQINGYDYY
YITLAATDDAGKKAIYEAKIGVVESAGWTGVEEFKLV
h2t2

(SEQ ID NO: 307)

GIVNVPNCNTTKYQQLARIAVAIYNYHEQAHLTFVENLNCKEQLGSGTYY
YITLAATDDAGKKAIYEAKIGVVESAGWTGVEEFKLV
pr_gen_pan5

(SEQ ID NO: 308)

GIVNVPNCNTTKYQRLADRAVAIYNYHEQAHLTFVENLNCKEQINGYDYY
YITLAATDDAGKKAIYEAKIGVVEPAGWVAVEEFKLV
gen_perm16

(SEQ ID NO: 309)

GIVNVPNCNTTKYQQLAQTAVAIYNYHEQAHLTFVENLNCKEQLTGYDYY
YITLAATDDAGKKAIYEAKIGVVESAGWTGVEEFKLV
gen_perm18

(SEQ ID NO: 310)

GIVNVPNCNTTKYQQLAQTAVAIYNYHEQAHLTFVENLNCKEQVGEGVYY
YITLAATDDAGKKAIYEAKIGVVESAGWTGVEEFKLV
pan_perm2

(SEQ ID NO: 311)

GIVNVPNCNTTKYQQLARTAVAIYNYHEQAHLTFVENLNCKEQWGLGIMY
YITLAATDDAGKKAIYEAKIGVVEPAGWVAVEEFKLV
gen_perm15

(SEQ ID NO: 312)

GIVNVPNCNTTKYQQLAQTAVAIYNYHEQAHLTFVENLNCKEQLGDGVYY
YITLAATDDAGKKAIYEAKIGVVESAGWTGVEEFKLV
gen_perm22

(SEQ ID NO: 313)

GIVNVPNCNTTKYQQLAQDAVAIYNYHEQAHLTFVENLNCKEQLTGYDYY
YITLAATDDAGKKAIYEAKIGVVESAGWTGVEEFKLV
gen_perm1

(SEQ ID NO: 314)

GIVNVPNCNTTKYQQLARTAVAIYNYHEQAHLTFVENLNCKEQLGEGDYY
YITLAATDDAGKKAIYEAKIGVVESAGWTGVEEFKLV
panb_perm1

(SEQ ID NO: 315)

GIVNVPNCNTTKYQQLARTAVAIYNYHEQAHLTFVENLNCKEQWGLGIRY
YITLAATDDAGKKAIYEAKIGVVEPAGWVAVMEFKLV
gen_perm23

(SEQ ID NO: 316)

GIVNVPNCNTTKYQQLAQDAVAIYNYHEQAHLTFVENLNCKEQINGYDYY
YITLAATDDAGKKAIYEAKIGVVESAGWTGVEEFKLV
pr_gen_gen3

(SEQ ID NO: 317)

GIVNVPNCNTTKYQRLADRAVAIYNYHEQAHLTFVENLNCKEQLGDGVYY
YITLAATDDAGKKAIYEAKIGVVESAGWTGVEEFKLV
gen_perm6

(SEQ ID NO: 318)

GIVNVPNCNTTKYQQLARTAVAIYNYHEQAHLTFVENLNCKEQVGEGVYY
YITLAATDDAGKKAIYEAKIGVVESAGWTGVEEFKLV
gen_perm19

(SEQ ID NO: 319)

GIVNVPNCNTTKYQQLAQDAVAIYNYHEQAHLTFVENLNCKEQLGEGDYY
YITLAATDDAGKKAIYEAKIGVVESAGWTGVEEFKLV
combs20

(SEQ ID NO: 320)

GIVNVPNCNTTKYQQLARSAVAIYNYHEQAHLTFVENLNCKEQLGEGDYY
YITLAATDDAGKKAIYEAKIGVVESAGWTGVEEFKLV
pan_perm10

(SEQ ID NO: 321)

GIVNVPNCNTTKYQQLAQDAVAIYNYHEQAHLTFVENLNCKEQWGLGIRY
YITLAATDDAGKKAIYEAKIGVVEPAGWVAVEEFKLV
h2_perm7

(SEQ ID NO: 322)

GIVNVPNCNTTKYQDLAQTAVAIYNYHEQAHLTFVENLNCKEQLTGYDYY
YITLAATDDAGKKAIYEAKIGVVESAGWFGVEEFKLV
pr_gen_pan4

(SEQ ID NO: 323)

GIVNVPNCNTTKYQRLADRAVAIYNYHEQAHLTFVENLNCKEQLTGYDYY
YITLAATDDAGKKAIYEAKIGVVEPAGWVAVEEFKLV
combs2

(SEQ ID NO: 324)

GIVNVPNCNTTKYQQLARSAVAIYNYHEQAHLTFVENLNCKEQISEGIYY
YITLAATDDAGKKAIYEAKIGVVESAGWIGVEEFKLV
pan_perm12

(SEQ ID NO: 325)

GIVNVPNCNTTKYQQLAQDAVAIYNHEQAHLTFVENLNCKEQLGGPDRYY
ITLAATDDAGKKAIYEAKIGVVEPAGWVAVEEFKLV
hkt1

(SEQ ID NO: 326)

GIVNVPNCNTTKYQQLARRAVAIYNYHEQAHLTFVENLNCKEQTNGDVYY
YITLAATDDAGKKAIYEAKIGVVESAGWGVSEFKLV
gen_perm2

(SEQ ID NO: 327)

GIVNVPNCNTTKYQQLARTAVAIYNYHEQAHLTFVENLNCKEQLGEGDRY
YITLAATDDAGKKAIYEAKIGVVESAGWTGVEEFKLV
gen_perm17

(SEQ ID NO: 328)

GIVNVPNCNTTKYQQLAQTAVAIYNYHEQAHLTFVENLNCKEQINGYDYY
YITLAATDDAGKKAIYEAKIGVVESAGWTGVEEFKLV
panb_perm2

(SEQ ID NO: 329)

GIVNVPNCNTTKYQQLARTAVAIYNYHEQAHLTFVENLNCKEQWGLGIMY
YITLAATDDAGKKAIYEAKIGVVEPAGWVAVMEFKLV
h2_perm11

(SEQ ID NO: 330)

GIVNVPNCNTTKYQDLAQTAVAIYNYHEQAHLTFVENLNCKEQVGEGVYY
YITLAATDDAGKKAIYEAKIGVVESAGWFGVEEFKLV
h2_perm4

(SEQ ID NO: 331)

GIVNVPNCNTTKYQDLAQTAVAIYNYHEQAHLTFVENLNCKEQLGEGDRY
YITLAATDDAGKKAIYEAKIGVVESAGWKGVEEFKLV
combs11

(SEQ ID NO: 332)

GIVNVPNCNTTKYQQLARRAVAIYNYHEQAHLTFVENLNCKEQTGFGNYY
YITLAATDDAGKKAIYEAKIGVVESAGWEGVSEFKLV
panb_perm10

(SEQ ID NO: 333)

GIVNVPNCNTTKYQQLAQDAVAIYNYHEQAHLTFVENLNCKEQWGLGIRY
YITLAATDDAGKKAIYEAKIGVVEPAGWVAVMEFKLV
combs23

(SEQ ID NO: 334)

GIVNVPNCNTTKYQQLARRAVAIYNYHEQAHLTFVENLNCKEQSGVGVYY
YITLAATDDAGKKAIYEAKIGVVESAGWVAVGEFKLV
combs10

(SEQ ID NO: 335)

GIVNVPNCNTTKYQQLARRAVAIYNYHEQAHLTFVENLNCKEQIGFGDYY
YITLAATDDAGKKAIYEAKIGVVESAGWEGVSEFKLV
pr_gen_gen1

(SEQ ID NO: 336)

GIVNVPNCNTTKYQRLADRAVAIYNYHEQAHLTFVENLNCKEQLGEGDYY
YITLAATDDAGKKAIYEAKIGVVESAGWTGVEEFKLV
combs18

(SEQ ID NO: 337)

GIVNVPNCNTTKYQQLARIAVAIYNYHEQAHLTFVENLNCKEQSGLGDYY
YITLAATDDAGKKATYEAKIGVVESAGWTGVEEFKLV
hkt2

(SEQ ID NO: 338)

GIVNVPNCNTTKYQQLARSAVAIYNYHEQAHLTFVENLNCKEQIGDGTYY
YITLAATDDAGKKAIYEAKIGVVESAGWEVVSEFKLV
h2_perm12

(SEQ ID NO: 339)

GIVNVPNCNTTKYQDLAQTAVAIYNYHEQAHLTFVENLNCKEQVGEGVYY
YITLAATDDAGKKAIYEAKTGVVESAGWKGVEEFKLV
pr_gen_pan1

(SEQ ID NO: 340)

GIVNVPNCNTTKYQRLADRAVAIYNYHEQAHLTFVENLNCKEQLGEGDYY
YITLAATDDAGKKAIYEAKIGVVEPAGWVAVEEFKLV
singt1

(SEQ ID NO: 341)

GIVNVPNCNTTKYQQLARRAVAIYNYHEQAHLTFVENLNCKEQTAGMGYY
YITLAATDDAGKKAIYEAKIGVVESAGWVAVGEFKLV
combs1

(SEQ ID NO: 342)

GIVNVPNCNTTKYQQLARIAVAIYNYHEQAHLTFVENLNCKEQISDGVYY
YITLAATDDAGKKAIYEAKIGVVESAGWTGVEEFKLV
gen_perm21

(SEQ ID NO: 343)

GIVNVPNCNTTKYQQLAQDAVAIYNYHEQAHLTFVENLNCKEQLGDGVYY
YITLAATDDAGKKAIYEAKIGVVESAGWTGVEEFKLV
h2_perm3

(SEQ ID NO: 344)

GIVNVPNCNTTKYQDLAQTAVAIYNYHEQAHLTFVENLNCKEQLGEGDRY
YITLAATDDAGKKAIYEAKIGVVESAGWFGVEEFKLV
combs14

(SEQ ID NO: 345)

GIVNVPNCNTTKYQQLARRAVAIYNYHEQAHLTFVENLNCKEQLTGYDYY
YITLAATDDAGKKAIYEAKIGVVESAGWEVVAEFKLV
pr_gen_gen5

(SEQ ID NO: 346)

GIVNVPNCNTTKYQRLADRAVAIYNYHEQAHLTFVENLNCKEQINGYDYY
YITLAATDDAGKKAIYEAKIGVVESAGWTGVEEFKLV

1. An isolated polypeptide comprising an amino acid sequence at least 90% identical over the full length of a polypeptide selected from the group consisting of SEQ ID NOS: 200-212 and SEQ ID NO:227.

2. The isolated polypeptide of claim 1 comprising an amino acid sequence at least 95% identical over the full length of a polypeptide selected from the group.

3. The isolated polypeptide of claim 1 comprising the amino acid sequence of a polypeptide selected from the group.

4. The isolated polypeptide of claim 1, further comprising an amino acid linker sequence at its C-terminus.

5.-7. (canceled)

8. A multimer, comprising two or more monomeric units of a polypeptide according to claim 1.

9. The multimer of claim 8, wherein the multimer comprises a trimer.

10. The multimer of claim 8, wherein the two or more monomeric units of the polypeptide are identical.

11. The isolated polypeptide of claim 1, wherein the isolated polypeptide further comprises a polypeptide receptor binding site (RBS) inhibitor selected from the group consisting of:
(a) a polypeptide at least 70% identical over the full length of the amino acid sequence of any one of SEQ ID NOS:1-5; and
(b) a polypeptide comprising the amino acid sequence of any one of SEQ ID NOS: 6-97, 121-199, and 228-346.

12. The isolated polypeptide of claim 1, wherein the isolated polypeptide further comprises a polypeptide receptor binding site (RBS) inhibitor selected from the group consisting of:
(a) a polypeptide at least 90% identical over the full length of the amino acid sequence of any one of SEQ ID NOS:1-5; and
(b) a polypeptide comprising the amino acid sequence of any one of SEQ ID NOS: 6-82, 85-89, 95-97, 125-128, 131-199, and 228-346.

13. The isolated polypeptide of claim 1, wherein the isolated polypeptide further comprises a polypeptide receptor binding site (RB S) inhibitor comprising the amino acid sequence of any one of SEQ ID NOS: 1-82, 85-89, 95-97, 125-128, 131-199, and 228-346.

14. The isolated polypeptide of claim 1, comprising (a) the isolated polypeptide of SEQ ID NO:200 (HAA.1) or 227 (HAA.1c) linked via an amino acid linker to (b) the isolated polypeptide of SEQ ID NO: 96 (52-NC2), 135 (Hsc1), or 141 (Hsc1-EA).

15. A multimer comprising two or more monomeric units of a polypeptide according to claim 11.

16. The multimer of claim 15, wherein the two or more monomeric units of the polypeptide are identical.

17. An isolated polypeptide having the amino acid sequence of SEQ ID NO:1, wherein one or more of the following is true:
(a) 1, 2, or all 3 of amino acids 44-46 are absent;
(b) amino acid 11 is K or P;
(c) amino acid 14 is I or R;
(d) amino acid 15 is K or R;
(e) amino acid 17 is G;
(f) amino acid 19 is Q or T;
(g) amino acid 22 is H, S, or Y;
(h) amino acid 24 is I, M, V, or W;
(i) amino acid 27 is D;
(j) amino acid 28 is D, E, K, W, or Y;
(k) amino acid 29 is D, E, or P;
(l) amino acid 30 is E;
(m) amino acid 33 is A, F, H, I, K, L, M, N, Q, R, S, T, or Y
(n) amino acid 36 is H or Q;
(o) amino acid 43 is K
(p) amino acid 47 is K;
(q) amino acid 49 is L, W, or Y;
(r) amino acid 51 is W;
(s) amino acid 59 is Q;
(t) amino acid 62 is E;
(u) amino acid 64 is A, G, L, M, Q, S, or W;
(v) amino acid 67 is A, F, M, S, W, or Y;
(w) amino acid 68 is T;
(x) amino acid 70 is L;
(y) amino acid 76 is S;
(z) amino acid 79 is L;
(aa) amino acid 81 is L;
(bb) amino acid 85 is A, D, or E; and/or
(cc) amino acid 87 is I or M

18. The isolated polypeptide of claim 17, wherein 1, 2, or all 3 of amino acids 44-46 are absent.

19. The isolated polypeptide of claim 17, comprising the amino acid sequence of a polypeptide selected from the group consisting of SEQ ID NOS: 77, 79, 135-199, and 228-346.

20. The isolated polypeptide of claim 1, further comprising a tag.

21. (canceled)

22. A pharmaceutical composition comprising the polypeptide of claim 10 and a pharmaceutically acceptable carrier.

23.-25. (canceled)

26. An assembly, comprising:
(a) a scaffold; and
(b) three hemagglutinin (HA) receptor binding site (RBS) inhibitors bound to the scaffold, wherein the scaffold organizes the three inhibitors such that each inhibitor is between about 40 A° and about 60 A° in distance from each other and at an angle of between about 57 degrees and about 63 degrees from each other.

27.-37. (canceled)

38. A method for treating and/or limiting an influenza infection, comprising administering to a subject in need thereof a therapeutically effective amount of the polypeptide of claim 1, or salts thereof, to treat and/or limit the influenza infection.

39.-40. (canceled)
